(19) United States
(12) Patent Application Publication
(10) Pub. No.: US 2024/0425515 A1
(43) Pub. Date: Dec. 26, 2024

(54) COMPOUND OF THE 7A,8,9,10,11,11A-HEXAHYDRO-1H,7H-PYRANO[2,3-C]XANTHENE TYPE, METHOD OF PREPARATION THEREOF, INTERMEDIATES THEREOF AND THERAPEUTIC APPLICATIONS THEREOF

(71) Applicants: CENTRE NATIONAL DE LA RECHERCHE SCIENTIFIQUE, Paris (FR); INSTITUT NATIONAL DE LA SANTE ET DE LA RECHERCHE MEDICALE, Paris (FR); UNIVERSITÉ CÔTE D'AZUR, Nice (FR)

(72) Inventors: Fanny ROUSSI, Gif-sur-Yvette (FR); Sandy DESRAT, Gif-sur-Yvette (FR); Gwenaëlle JEZEQUEL, Gif-sur-Yvette (FR); Jérôme BIGNON, Gif-sur-Yvette (FR); Marc LITAUDON, Gif-sur-Yvette (FR); Bruno ANTONNY, Valbonne (FR); Joëlle BIGAY, Valbonne (FR); Bruno MESMIN, Valbonne (FR)

(21) Appl. No.: 18/577,572

(22) PCT Filed: Jul. 4, 2022
(86) PCT No.: PCT/EP2022/068380

§ 371 Date: Jan. 8, 2024

(30) Foreign Application Priority Data
Jul. 6, 2021 (FR) FR2107303

(51) Int. Cl. C07D 493/04; A61K 31/352

(52) U.S. Cl. CPC C07D 493/04; A61K 31/352

(57) ABSTRACT

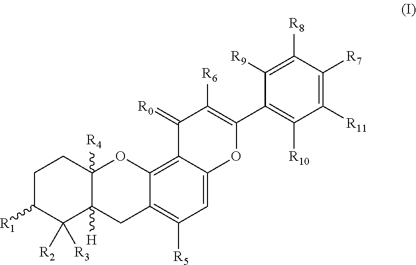

Compound of the 7a,8,9,10,11,1la-hexahydro-1H,7H-pyrano[2,3-c]xanthene type, method of preparation thereof, intermediates thereof and therapeutic applications thereof. The present invention relates to compounds of formula (I) in which R₀ represents O or N—ORₐ, R₁ is OH or protected OH, R₂ R₃ R₄ represent, independently, a C₁-C₄ alkyl, R₅ represents OH, a protected OH, —O—P(═O)(OH)₂, a sugar, H, a halogen, —CF₃, a C₁-C₄ alkyl, a C₂-C₄ alkenyl or a C₂-C₄ alkynyl, R₆ represents H, OH, protected OH, —O—P(═O)(OH)₂, or a sugar, R₇ represents H, OH, protected OH, a halogen, a C₁-C₄ alkyl, a C₂-C₄ alkenyl, a C₂-C₄ alkynyl, a triazolyl, —O—Rb, N(Rc)(Rd), —C(═O)—N(Re)(Rf), —O—C(═O)—N(Rg)(Rh), and R₈, R₉, R₁₀, and R₁₁ are, independently, H, OH or protected OH. The present invention further relates to the method of preparation thereof, the intermediates thereof and the therapeutic applications thereof.

### TECHNICAL FIELD

The present invention relates to the pharmaceutical field. More particularly, it relates to new compounds of the 7a,8,9,10,11,11a-hexahydro-1H,7H-pyrano[2,3-c]xanthene type, the method of preparation thereof, pharmaceutical compositions comprising them as well as the use thereof as medication, in particular as inhibitors of OSBP (oxysterol-binding protein) for the prevention and/or treatment in particular of cancers, viral diseases, and neurodegenerative diseases. It further relates to new intermediates.

### PRIOR ART

It has recently been demonstrated that OSBP is implicated in certain diseases, in particular in Antonietta Pietrangelo et al., “Bridging the molecular and biological functions of the oxysterol-binding protein family”, Cellular and Molecular Life Sciences, Springer International Publishing AG, part of Springer Nature 2018.

Molecules such as the schweinfurthins, which are classed among the ORPhilines and are natural products isolated from plants of the genus Macaranga (Euphorbiaceae), are already known for use as OSBP inhibitors (Péresse et al., Molecular and cellular dissection of the OSBP cycle through a fluorescent inhibitor, Nov. 2019). The schweinfurthins display powerful, selective cytotoxic activity on the panel of 60 human cancer cell lines of the National Cancer Institute (NCI). They are particularly active against the lines of glioblastoma, kidney and certain leukemias (acute lymphoblastic leukemias or myelomas). Their cytotoxicity profile is not at all similar to the profiles of the molecules currently used in anticancer chemotherapy, which indicates that they act via a new biological target. This targeted biological activity makes the schweinfurthins attractive as they are the leaders of a new series of original anticancer agents.

The document Dipesh S. Harmalkar et al., “Schweinfurthins A-Q: isolation, synthesis, and biochemical properties”, RSC. Adv., 2018, 8, 21191-21209, discloses various derivatives of schweinfurthins, including schweinfurthin G (SWG), as well as their vegetable origin. However, the schweinfurthins are still difficult to access, whether by extraction from the organisms from which they are derived or by chemical synthesis. Moreover, their complete synthesis comprises twenty steps on average.

Other complex natural compounds such as stelletin E, OSW1 (also called YOR255W), cephalostatin and ritterazine B also display the property of inhibiting OSBP, but they are difficultly accessible as they are obtained by extraction with a low or even very low yield, and their complete synthesis is complex and often long. Moreover, their high molecular weight may be an obstacle to therapeutic development.

### DISCLOSURE OF THE INVENTION

The main types of treatment against cancer are surgery, radiotherapy, so-called conventional chemotherapy (involving cytotoxic agents), targeted therapies (aimed specifically at certain mechanisms involved in cell regulation and growth), hormonotherapy (adapted in the case of cancers sensitive to the action of hormones produced by the body naturally), and immunotherapy (aiming to stimulate the patient's immune system against tumor cells).

More particularly, chemotherapy, which is still one of the most effective treatments, is based on a cytotoxic effect: a molecule kills the cancerous cells, in order to stop tumor growth.

However, these treatments give rise to severe side effects for the patients on account of the lack of selectivity, which leads in particular to damage to healthy cells. In order to limit these effects, the drugs are often dosed at minimal levels, to the detriment of their efficacy. Consequently it is still important to find a means of targeting tumor cells more specifically. Moreover, the risk of emergence of new viral diseases such as dengue, infection with Zika virus, or infection with SARS-CoV-2 virus (coronavirus 2 of severe acute respiratory syndrome) is still worrying. In fact, nowadays, there are as many imported cases as indigenous cases for these types of diseases, the latter implying that the virus is circulating in the territory in question. Although most of the time these diseases are asymptomatic, a proportion of persons that is not negligible (20-30%) develops flu-like symptoms (fever, headaches, joint and muscle pains, or even skin rash), which may lead to complications that are sometimes severe.

Furthermore, neurodegenerative diseases such as Alzheimer's, Parkinson's, Huntington's or Niemann-Pick type C are of frequent occurrence, and their frequency increases with age. Because of the progressive aging of the population and absence of therapeutic treatments, the number of persons suffering from neurodegenerative diseases has increased considerably in recent decades and is expected to grow regularly in the years to come.

There is therefore still a need for new stable compounds that display effective inhibitory action against OSBP and that can be prepared by a method comprising a reasonable number of steps, which employs readily accessible starting reagents while making it possible to obtain the compounds with good yields.

The invention aims to meet these needs.

### SUMMARY OF THE INVENTION

According to one of its aspects, the present invention relates to a new compound of formula (I), the pharmaceutically acceptable salts thereof, its hydrates, its solvates, as well as its tautomeric forms, stereoisomers, mixtures of stereoisomers, pure enantiomers and mixtures, whether or not racemic

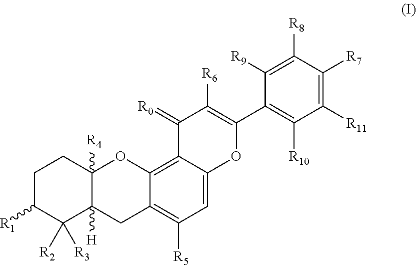

in which
R₀ represents an oxygen atom or a group N—ORₐ,
R₁ represents a hydroxyl group or a protected hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group, a protected hydroxyl group, a group —O—P(═O)(OH)₂, a sugar radical, a hydrogen atom, a halogen atom, a —CF₃ group, a linear or branched C₁-C₄ alkyl group, a linear or branched C₂-C₄ alkenyl group or a linear or branched C₂-C₄ alkynyl group,
R₆ represents a hydrogen atom, a hydroxyl group, a protected hydroxyl group, a group —O—
P(═O)(OH)₂, or a sugar radical,
R₇ represents a hydrogen atom, a hydroxyl group, a protected hydroxyl group, a halogen atom, a linear or branched C₁-C₄ alkyl group, a linear or branched C₂-C₄ alkenyl group, a linear or branched C₂-C₄ alkynyl group, a triazolyl group, a group —O—Rb, a group N(Rc)(Rd), a group —C(═O)—N(Re)(Rf), —O—C(═O)—N(Rg)(Rh),
R₈ represents a hydrogen atom, a hydroxyl group or a protected hydroxyl group,
R₉ represents a hydrogen atom, a hydroxyl group or a protected hydroxyl group,
R₁₀ represents a hydrogen atom, a hydroxyl group or a protected hydroxyl group,
R₁₁ represents a hydrogen atom, a hydroxyl group or a protected hydroxyl group,
Ra, Rb, Rc, Rd, Re, Rf, Rg and Rh represent, independently of one another, a hydrogen atom or a linear or branched C₁-C₄ alkyl group, and

the symbol [symbol] represents a single bond joining an asymmetric carbon atom to the group or atom in question, this bond being either in front of or behind the plane.

According to another of its aspects, the present invention also relates to a compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to the present invention or a pharmaceutical composition according to the present invention for use as a drug.

According to another of its aspects, the present invention also relates to a compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to the present invention or pharmaceutical composition according to the present invention for use as an inhibitor of OSBP (oxysterol-binding protein).

According to another of its aspects, the present invention also relates to a method for preparing a compound of formula (I), as well as the pharmaceutically acceptable salts thereof, its hydrates, its solvates, as well as its tautomeric forms, stereoisomers, mixtures of stereoisomers, pure enantiomers and mixtures, whether or not racemic, as defined according to the present invention comprising at least the following steps:
(i) Cyclization of a compound of formula (VII)

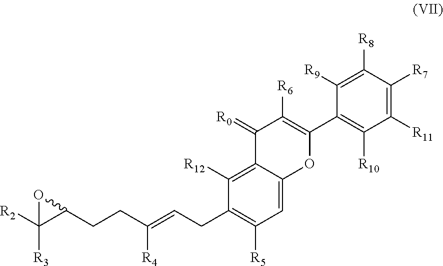

in which

R₀, and R₂ to R₁ are as defined according to the present invention and R₁₂ represents a hydroxyl group or a protected hydroxyl group, provided that at least one of R₅ and R₁₂ is a protected hydroxyl group, and the symbol [symbol] represents a single bond joining an asymmetric carbon atom to the group or atom in question, this bond being either in front of or behind the plane, to obtain a compound of formula (I) as defined according to the present invention, and

(ii) Optionally deprotection of at least one hydroxyl group remaining protected by a protective group present in said compound obtained in step (i), to obtain a compound of formula (I) as defined according to the present invention.

According to another of its aspects, the present invention further relates to a pharmaceutical composition comprising at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to the present invention, and at least one pharmaceutically acceptable excipient.

Finally, according to another of its aspects, the present invention relates to intermediates of formulas (IV), (V), (VI) and (VII), their pharmaceutically acceptable salts, their hydrates, their solvates, as well as their tautomeric forms, stereoisomers, mixtures of stereoisomers, pure enantiomers and mixtures, whether or not racemic:

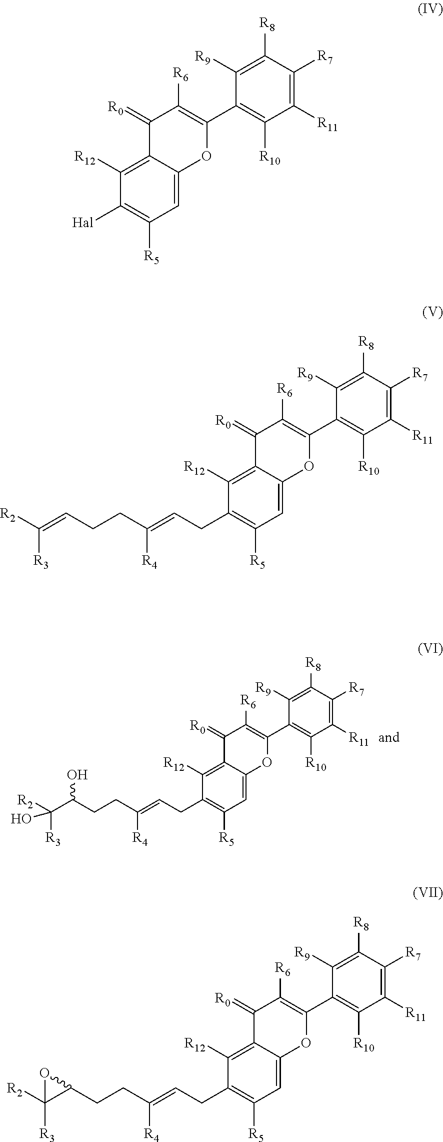

in which R₀, R₂, R₃, R₄, Hal, R₅, R₆, R₇, R₈, R₉, R₁₀, R₁₁ and R₁₂ are as defined in the present invention, provided that at least one of R₅ and R₁₂ is a protected hydroxyl group, Hal is a halogen atom, and the symbol [symbol] represents a single bond joining an asymmetric carbon atom to the group or atom in question, this bond being either in front of or behind the plane.

### DETAILED DESCRIPTION

The inventors have discovered, surprisingly, that new compounds of the 7a,8,9,10,11,11a-hexahydro-1H,7H-pyrano[2,3-c]xanthene type can be used as inhibitors of OSBP and can be prepared from commercially available natural reagents in a limited number of steps.

In fact, as demonstrated in the experimental section, advantageously, the method of preparation (complete synthesis) according to the invention of the compounds of formula (I) as defined in the invention, comprises only seven steps.

Moreover, the method according to the invention may be carried out exclusively starting from commercially available natural compounds, which has appreciable advantages in particular in terms of supply.

As shown in the examples, the inventors have moreover validated the cytotoxic activity on cell lines, and more particularly on lines U87 and A549. The U87 are cells of a human cancer cell line of glioblastomas (GBM). The cells A549 are human adenocarcinoma basal alveolar epithelial cells and constitute a cell line of choice for validating cytotoxic activity.

Furthermore, in the experimental section, the inventors have shown that compounds of formula (I) according to the invention have affinity for the target OSBP, are stable in the plasma, and have good microsomal stability.

Other characteristics, aspects and advantages of the invention will become clearer on reading the detailed description given hereunder.

### Definitions

“Room temperature” means, in the present invention, a temperature between 18° C. and 30° C., preferably between 18° C. and 25° C.
In the context of the invention:
“Alkyl” means a linear or branched saturated aliphatic group; for example, a Cx to Cz alkyl represents a linear or branched hydrocarbon chain with x to z carbon atoms. We may mention in particular a methyl group, an ethyl group, a propyl group, an isopropyl group, an n-butyl group, an isobutyl group, a tert-butyl group, a sec-butyl group.
“Alkenyl” means a linear or branched unsaturated aliphatic group, comprising at least one double bond; for example, a Cx to Cz alkenyl group represents a linear or branched unsaturated carbon chain with x to z carbon atoms. We may mention in particular a vinyl group, vinyl, 1-propenyl, 2-propenyl and butenyl.
“Alkynyl” means a linear or branched unsaturated aliphatic group, comprising at least one triple bond; for example, a Cx to Cz alkynyl group represents a linear or branched unsaturated carbon chain with x to z carbon atoms. We may mention in particular an ethynyl group or a propynyl group.
“Protected hydroxyl group” means a hydroxyl group whose hydrogen atom has been replaced with a protective group conventionally used by a person skilled in the art. We may mention in particular —O—CH₂—O—CH₃, a methoxy group, —O—C(═O)—CH₃, a tert-butyldimethylsilyloxy group, a benzyloxymethoxy group, a benzyloxy group, a trityloxy group, a para-methoxybenzyloxy group, a trimethylsilyloxy group, a tert-butyldiphenylsilyloxy group, a triisopropylsilyloxy group, a pivaloyloxy group.
“Protective group” means a functional group introduced into the molecule from a chemical function for masking all or part of its reactivity.
“Halogen atom” means a fluorine, chlorine, bromine or iodine atom, preferably an iodine atom.
“Unsaturated aliphatic group” means a hydrocarbon chain that may comprise one or more unsaturations.
“Unsaturation” means a double bond or a carbon-carbon triple bond.
“Sugar radical” denotes a furanose radical or hexose radical, natural or nonnatural. The nonnatural sugar radicals may for example comprise hydroxyl and/or amine residues alkylated or acylated on the ring, such as ether, ester and amide substituents. Other nonnatural sugar radicals may comprise H, hydroxyl, ether, ester or amide substituents at positions on the ring where these substituents are not present in the natural sugars. Alternatively, the sugar radical does not comprise a substituent at its usual position on natural sugar; this particular case corresponds to the deoxy sugar radical. Other examples of nonnatural sugars comprise the oxidized carbohydrates (for example, the -onic and-uronic acids) and reduced carbohydrates (sugar alcohol). The sugar residue may be a monosaccharide, an oligosaccharide or a polysaccharide. As natural sugar radicals usable in the context of the present invention we may mention glucose, galactose, fucose, mannose, xylanose, ribose, N-acetylglucose (GlcNAc), sialic acid and N-acetylgalactose (GalNAc).
The expression “pharmaceutically acceptable” denotes what is useful in the preparation of a pharmaceutical composition, which is generally safe, nontoxic and neither biologically nor otherwise undesirable and that is acceptable for veterinary and/or human pharmaceutical use.
“Pharmaceutically acceptable salt” of a compound is intended to denote, in the present invention, salts that are pharmaceutically acceptable, as defined herein, and that possess the desired pharmacological activity of the parent compound. Such salts comprise: (1) the salts of acid addition formed with inorganic acids such as hydrochloric acid, hydrobromic acid, sulfuric acid, nitric acid, phosphoric acid and similar; or formed with organic acids such as acetic acid, benzenesulfonic acid, benzoic acid, camphorsulfonic acid, citric acid, ethane-sulfonic acid, fumaric acid, gluconic acid, glutamic acid, glycolic acid, 2-hydroxyethanesulfonic acid, lactic acid, maleic acid, malic acid, 2-naphthalenesulfonic acid, propionic acid, salicylic acid, succinic acid, tartaric acid, p-toluenesulfonic acid, trifluoroacetic acid and similar; and (2) the salts formed when an acid proton present in the parent compound either is replaced with a metal ion, for example an alkali metal ion (Na+, K+ or Li+ for example), an alkaline-earth metal ion (such as Ca²+ or Mg²+) or an aluminum ion; or is coordinated with an organic or inorganic base. The acceptable organic bases comprise diethanolamine, ethanolamine, N-methylglucamine, triethanolamine, tromethamine and similar. The acceptable inorganic bases comprise aluminum hydroxide, calcium hydroxide, potassium hydroxide, sodium carbonate and sodium hydroxide.
“Hydrate or solvate” means forms of associations or of combinations with one or more water molecules or with a solvent.

The isomeric forms denote racemates (also called racemic mixtures), enantiomers, diastereoisomers, epimers, tautomers as well as conformation isomers. A racemic form denotes a mixture of two enantiomers in a ratio from 55:45 to 45:55.

### Compounds of Formula (I) According to the Invention

The present invention thus relates to a compound of formula (I), the pharmaceutically acceptable salts thereof, its hydrates, its solvates, as well as its tautomeric forms, stereoisomers, mixtures of stereoisomers, pure enantiomers and mixtures, whether or not racemic

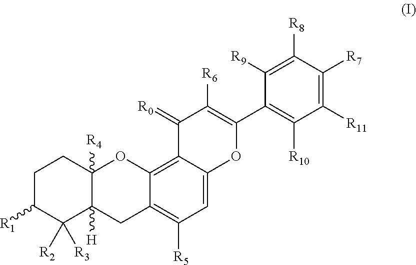

in which
R₀ represents an oxygen atom or a group N—ORₐ,
R₁ represents a hydroxyl group or a protected hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group, a protected hydroxyl group, a group —O—P(═O)(OH)₂, a sugar radical, a hydrogen atom, a halogen atom, a —CF₃ group, a linear or branched C₁-C₄ alkyl group, a linear or branched C₂-C₄ alkenyl group or a linear or branched C₂-C₄ alkynyl
group, R₆ represents a hydrogen atom, a hydroxyl group, a protected hydroxyl group, a group —O—P(═O)(OH)₂, or a sugar radical,
R₇ represents a hydrogen atom, a hydroxyl group, a protected hydroxyl group, a halogen atom, a linear or branched C₁-C₄ alkyl group, a linear or branched C₂-C₄ alkenyl group, a linear or branched C₂-C₄ alkynyl group, a triazolyl group, a group —O—Rb, a group N(Rc)(Rd), a group —C(═O)—N(Re)(Rf), —O—C(═O)—N(Rg)(Rh),
R₈ represents a hydrogen atom, a hydroxyl group or a protected hydroxyl group,
R₉ represents a hydrogen atom, a hydroxyl group or a protected hydroxyl group,
R₁₀ represents a hydrogen atom, a hydroxyl group or a protected hydroxyl group,
R₁₁ represents a hydrogen atom, a hydroxyl group or a protected hydroxyl group, Ra, Rb, Rc, Rd, Re, Rf, Rg and Rh represent, independently of one another, a hydrogen atom or a linear or branched C₁-C₄ alkyl group, and the symbol [symbol] represents a single bond joining an asymmetric carbon atom to the group or atom in question, this bond being either in front of or behind the plane.

According to a particular embodiment, R₀ represents an oxygen atom.

According to a particular embodiment, R₁ represents a hydroxyl group or a group —O—CH₂—O—CH₃.

According to a particular embodiment, R₂ represents a methyl group.

According to a particular embodiment, R₃ represents a methyl group.

According to a particular embodiment, R₄ represents a methyl group.

According to a particular embodiment, R₅ represents a hydroxyl group or a group —O—CH₂—O—CH₃.

According to a particular embodiment, R₆ represents a hydrogen atom, a hydroxyl group, or a group —O—CH₂—O—CH₃.

According to a particular embodiment, R₇ represents a hydrogen atom, a hydroxyl group, or a group —O—CH₂—O—CH₃.

According to a particular embodiment, R₈ represents a hydrogen atom.

According to a particular embodiment, R₉ represents a hydrogen atom.

According to a particular embodiment, R₁₀ represents a hydrogen atom.

According to a particular embodiment, R₁₁ represents a hydrogen atom.

According to a particularly preferred embodiment of the invention, the protected hydroxyl group is selected from —O—CH₂—O—CH₃, a methoxy group, —O—C(═O)—CH₃, a tert-butyldimethylsilyloxy group, a benzyloxymethoxy group, a benzyloxy group, a trityloxy group, a para-methoxybenzyloxy group, a trimethylsilyloxy group, a tert-butyldiphenylsilyloxy group, a triisopropylsilyloxy group, and a pivaloyloxy group, is preferably selected from —O—CH₂—O—CH₃, a methoxy group, —O—C(═O)—CH₃, a tert-butyldimethylsilyloxy group, and a benzyloxymethoxy group, more preferably is —O—CH₂—O—CH₃.

According to another particularly preferred embodiment of the invention:
R₀ represents an oxygen atom,
R₁ represents a hydroxyl group or a protected hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a protected hydroxyl group,
R₆ represents a hydrogen atom, a hydroxyl group or a protected hydroxyl group,
R₇ represents a hydrogen atom, a hydroxyl group or a protected hydroxyl group, and
R₈, R₉, R₁₀, and R₁₁ each represent a hydrogen atom.

According to another particularly preferred embodiment of the invention:
R₀ represents an oxygen atom
R₁ represents a hydroxyl group or a group —O—CH₂—O—CH₃,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a group —O—CH₂—O—CH₃,
R₆ represents a hydrogen atom, a hydroxyl group or a group —O—CH₂—O—CH₃,
R₇ represents a hydrogen atom, a hydroxyl group or a group —O—CH₂—O—CH₃, and
R₈, R₉, R₁₀, and R₁₁ each represent a hydrogen atom.

According to another particularly preferred embodiment of the invention:
R₀ represents an oxygen atom,
R₁ represents a hydroxyl group or a group —O—CH₂—O—CH₃,
R₂ represents a methyl group,
R₃ represents a methyl group,
R₄ represents a methyl group,
R₅ represents a hydroxyl group or a group —O—CH₂—O—CH₃,
R₆ represents a hydrogen atom, a hydroxyl group or a group —O—CH₂—O—CH₃,
R₇ represents a hydrogen atom, a hydroxyl group or a group —O—CH₂—O—CH₃, and
R₈, R₉, R₁₀, and R₁₁ each represent a hydrogen atom.

According to another particularly preferred embodiment of the invention:
R₀ represents an oxygen atom,
R₁ represents a hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a protected hydroxyl group,
R₆ represents a hydrogen atom, and
R₇, R₈, R₉, R₁₀, and R₁₁ each represent a hydrogen atom.

According to another particularly preferred embodiment of the invention:
R₀ represents an oxygen atom,
R₁ represents a hydroxyl group,
R₂ represents a methyl group,
R₃ represents a methyl group,
R₄ represents a methyl group,
R₅ represents a hydroxyl group or a group —O—CH₂—O—CH₃,
R₆ represents a hydrogen atom, and
R₇, R₈, R₉, R₁₀, and R₁₁ each represent a hydrogen atom.

The compounds of formula (I) may in particular be selected from the following compounds shown in Tables 1 and 2, characterized by NMR (nuclear magnetic resonance) and/or by mass spectrometry.

TABLE 1

| Number of the compound and | |
|---|---|
| chemical name | Structure of the compound |
| 1 (7aR*9R*,11aR*)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (+/− racemic mixture) | 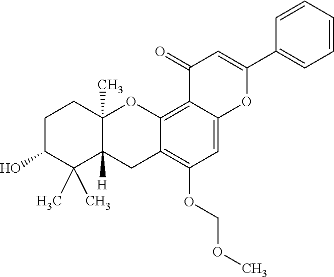 [SMILES: COCOc1cc2oc(-c3ccccc3)cc(=O)c2c2c1C[C@@H]1C(C)(C)[C@H](O)CC[C@@]1(C)O2] |
| 2 (7aR*,9R*,11aR*)-6,9- bis(methoxymethoxy)-8,8,11a- trimethyl-3-phenyl- 7a,8,9,10,11,11a-hexahydro- 1H,7H-pyrano[2,3-c]xanthen-1- one (+/− racemic mixture) | 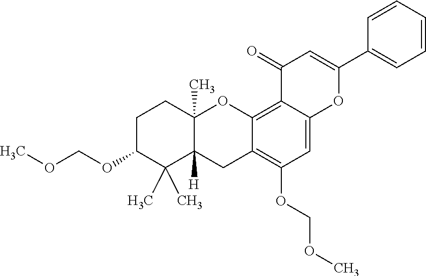 [SMILES: COCOc1cc2oc(-c3ccccc3)cc(=O)c2c2c1C[C@@H]1C(C)(C)[C@H](OCOC)CC[C@@]1(C)O2] |
| 3 (7aR*,9R*,11aR*)-6,9-dihydroxy- 8,8,11a-trimethyl-3-phenyl- 7a,8,9,10,11,11a-hexahydro- 1H,7H-pyrano[2,3-c]xanthen-1- one (+/− racemic mixture) | 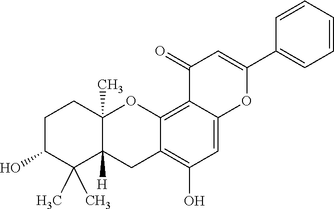 [SMILES: CC1(C)[C@H](O)CC[C@@]2(C)Oc3c(c(O)cc4oc(-c5ccccc5)cc(=O)c34)C[C@H]12] |
| 4 (7aR,9R,11aR)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (+) | 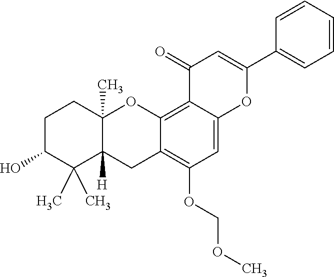 [SMILES: COCOc1cc2oc(-c3ccccc3)cc(=O)c2c2c1C[C@@H]1C(C)(C)[C@H](O)CC[C@@]1(C)O2] |
| 5 (7aR,9R,11aR)-6,9-dihydroxy- 8,8,11a-trimethyl-3-phenyl- 7a,8,9,10,11,11a-hexahydro- 1H,7H-pyrano[2,3-c]xanthen-1- one (+) | 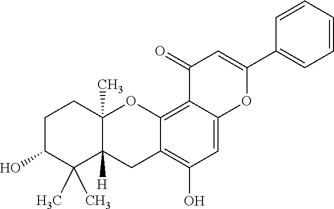 [SMILES: CC1(C)[C@H](O)CC[C@@]2(C)Oc3c(c(O)cc4oc(-c5ccccc5)cc(=O)c34)C[C@H]12] |
| 6 (7aS,9S,11aS)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (−) | 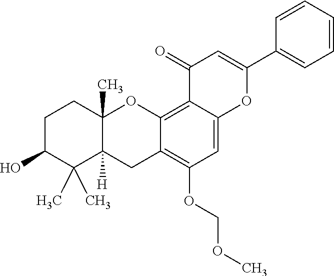 [SMILES: COCOc1cc2oc(-c3ccccc3)cc(=O)c2c2c1C[C@H]1C(C)(C)[C@@H](O)CC[C@]1(C)O2] |
| 7 (7aS,9S,11aS)-6,9-dihydroxy- 8,8,11a-trimethyl-3-phenyl- 7a,8,9,10,11,11a-hexahydro- 1H,7H-pyrano[2,3-c]xanthen-1- one (−) | 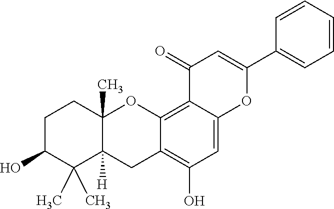 [SMILES: CC1(C)[C@@H](O)CC[C@]2(C)Oc3c(c(O)cc4oc(-c5ccccc5)cc(=O)c34)C[C@@H]12] |
| 8 (7aR,9R,11aR)-9-hydroxy-6- (methoxymethoxy)-3-(4- (methoxymethoxy)phenyl)- 8,8,11a-trimethyl- 7a,8,9,10,11,11a-hexahydro- 1H,7H-pyrano[2,3-c]xanthen-1- one (+) | 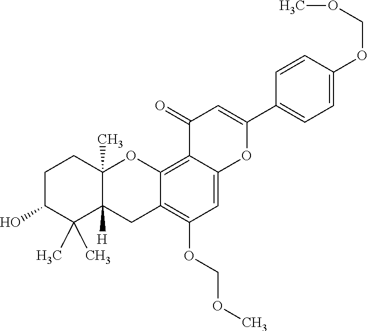 [SMILES: COCOc1ccc(-c2cc(=O)c3c4c(c(OCOC)cc3o2)C[C@@H]2C(C)(C)[C@H](O)CC[C@@]2(C)O4)cc1] |
| 9 (7aR,9R,11aR)-6,9-dihydroxy-3- (4-hydroxyphenyl)-8,8,11a- trimethyl-7a,8,9,10,11,11a- hexahydro-1H,7H-pyrano[2,3- c]xanthen-1-one (+) | 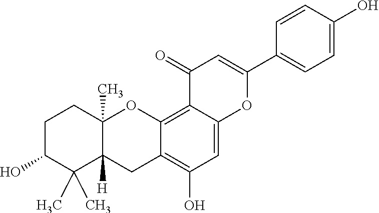 [SMILES: CC1(C)[C@H](O)CC[C@@]2(C)Oc3c(c(O)cc4oc(-c5ccc(O)cc5)cc(=O)c34)C[C@H]12] |
| 10 (7aR,9R,11aR)-9-hydroxy-2,6- bis(methoxymethoxy)-3-(4- (methoxymethoxy)phenyl). 8,8,11a-trimethyl- 7a,8,9,10,11,11a-hexahydro- 1H,7H-pyrano[2,3-c]xanthen-1- one (+) | 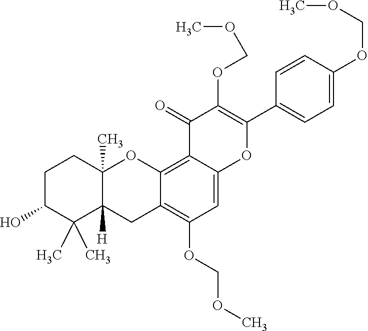 [SMILES: COCOc1ccc(-c2oc3cc(OCOC)c4c(c3c(=O)c2OCOC)O[C@]2(C)CC[C@@H](O)C(C)(C)[C@H]2C4)cc1] |
| 11 (7aR,9R,11aR)-2,6,9-trihydroxy-3- (4-hydroxyphenyl)-8,8,11a- trimethyl-7a,8,9,10,11,11a- hexahydro-1H,7H-pyrano[2,3- c]xanthen-1-one (+) | 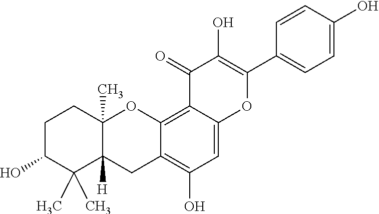 [SMILES: CC1(C)[C@H](O)CC[C@@]2(C)Oc3c(c(O)cc4oc(-c5ccc(O)cc5)c(O)c(=O)c34)C[C@H]12] |
| 12 (7aS*9R*,11aR*)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (+/− racemic mixture) | 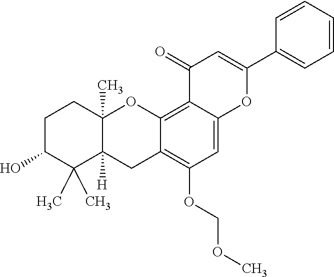 [SMILES: COCOc1cc2oc(-c3ccccc3)cc(=O)c2c2c1C[C@H]1C(C)(C)[C@H](O)CC[C@@]1(C)O2] |
| 13 (7&S,9R,11aR)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (+) | 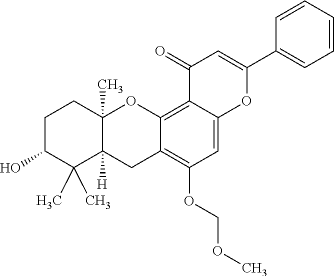 [SMILES: COCOc1cc2oc(-c3ccccc3)cc(=O)c2c2c1C[C@H]1C(C)(C)[C@H](O)CC[C@@]1(C)O2] |
| 14 (7aR,9S,11aS)-9-hydroxy-6- (methoxymethoxy)-8,8,11a- trimethyl-3-phenyl- 7a,8,9,10,11,11a-hexahydro- 1H,7H-pyrano[2,3-c]xanthen-1- one (−) | 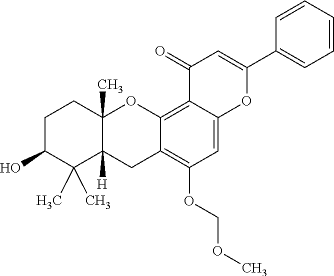 [SMILES: COCOc1cc2oc(-c3ccccc3)cc(=O)c2c2c1C[C@@H]1C(C)(C)[C@@H](O)CC[C@]1(C)O2] |
| 15 (7aS,9R,11aR)-9-hydroxy-2,6- bis(methoxymethoxy)-3-(4- (methoxymethoxy)phenyl)- 8,8,11a-trimethyl- 7a,8,9,10,11,11a-hexahydro- 1H,7H-pyrano[2,3-c]xanthen-1- one (+) | 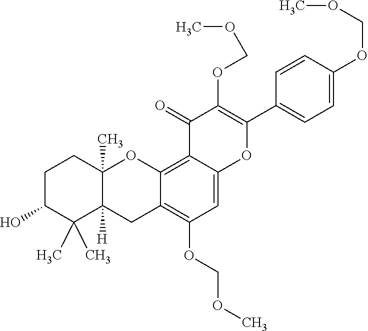 [SMILES: COCOc1ccc(-c2oc3cc(OCOC)c4c(c3c(=O)c2OCOC)O[C@]2(C)CC[C@@H](O)C(C)(C)[C@@H]2C4)cc1] |
| 16 (7aS,9S,11aS)-9-hydroxy-2,6- bis(methoxymethoxy)-3-(4- (methoxymethoxy)phenyl)- 8,8,11a-trimethyl- 7a,8,9,10,11,11a-hexahydro- 1H,7H-pyrano[2,3-c]xanthen-1- one (−) | 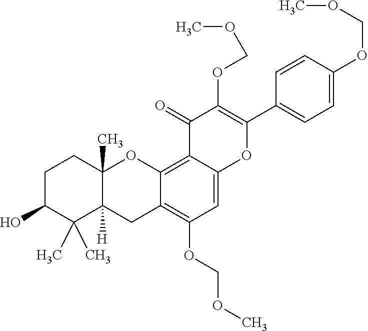 [SMILES: COCOc1ccc(-c2oc3cc(OCOC)c4c(c3c(=O)c2OCOC)O[C@@]2(C)CC[C@H](O)C(C)(C)[C@@H]2C4)cc1] |
| 17 (7aS,9S,11aS)-2,6,9-trihydroxy-3- (4-hydroxyphenyl)-8,8,11a- trimethyl-7a,8,9,10,11,11a- hexahydro-1H,7H-pyrano[2,3- c]xanthen-1-one (−) | 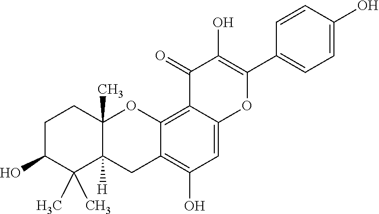 [SMILES: CC1(C)[C@@H](O)CC[C@]2(C)Oc3c(c(O)cc4oc(-c5ccc(O)cc5)c(O)c(=O)c34)C[C@@H]12] |
| 18 (7aS,9R,11aR)-9-hydroxy-6- (methoxymethoxy)-3-(4- (methoxymethoxy)phenyl)- 8,8,11a-trimethyl- 7a,8,9,10,11,11a-hexahydro- 1H,7H-pyrano[2,3-c]xanthen-1- one (+) | 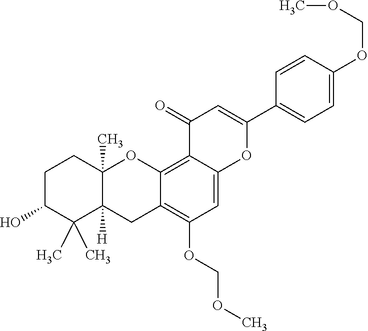 [SMILES: COCOc1ccc(-c2cc(=O)c3c4c(c(OCOC)cc3o2)C[C@H]2C(C)(C)[C@H](O)CC[C@@]2(C)O4)cc1] |

TABLE 2

| | Empirical | |
|---|---|---|
| Compound No. | formula | Characterization (NMR and MS) |
| 1 | C₂₇H₃₀O₆ | ¹H-NMR (500 MHz, CD₃CN): δ = 7.94 (dd, 2 H, J = 7.6 Hz), |
| (+/− racemic | | 7.55-7.51 (m, 3 H), 6.80 (s, 1 H), 6.51 (s, 1 H), 5.33 (s, 2 H), 3.50 |
| mixture) | | (s, 3 H), 3.39-3.33 (m, 1 H), 2.78 (dd, 1 H, J = 17.0, 5.1 Hz), 2.76 |
| | | (s, 1 H), 2.45 (dd, 1 H, J = 17.0, 13.4 Hz), 2.03 (dt, 1 H, J = 12.1, |
| | | 3.0 Hz), 1.82-1.74 (m, 2 H), 1.64-1.58 (m, 2 H), 1.21 (s, 3 H), |
| | | 1.07 (s, 3 H), 0.86 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 78.6 (1 C), 78.0 (1 C), 57.0 (1 C), 46.5 (1 |
| | | C), 39.3 (1 C), 38.6 (1 C), 29.1 (1 C), 27.7 (1 C), 20.2 (1 C), 18.9 |
| | | (1 C), 14.9 (1 C) ppm. |
| | | HRMS (ESI): m/z calculated for C₂₇H₃₁O₆⁺ [M + H]⁺: 451.2115; |
| | | found: 451.2115. |
| 2 | C₂₉H₃₄O₇ | ¹H-NMR (500 MHz, CD₃CN): δ = 7.94 (dd, 2 H, J = 7.6 Hz), |
| (+/− racemic | | 7.55-7.51 (m, 3 H), 6.80 (s, 1 H), 6.52 (s, 1 H), 5.33 (s, 2 H), 4.61 |
| mixture) | | (d, 1 H, J = 6.7 Hz), 4.74 (d, 1 H, J = 6.7 Hz), 3.50 (s, 3 H), 3.35 |
| | | (s, 3 H), 3.32 (dd, 1 H, J = 12.1, 5.2 Hz), 2.78 (dd, 1 H, J = 17.0, |
| | | 5.1 Hz), 2.45 (dd, 1 H, J = 17.0, 13.4 Hz), 2.03 (dt\|, 1 H, J = |
| | | 12.1, 3.0 Hz), 1.82-1.74 (m, 2 H), 1.64-1.58 (m, 2 H), 1.22 (s, 3 |
| | | H1.10 (s, 3 H), 0.91 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 97.1 (1 C), 95.4 (1 C), 94.6 (1 C), 84.8 (1 C), 78.3 (1 C), 57.0 |
| | | (1 C), 55.9 (1 C), 46.7 (1 C), 39.2 (1 C), 38.4 (1 C), 27.7 (1 C), |
| | | 26.1 (1 C), 20.2 (1 C), 18.8 (1 C), 15.6 (1 C) ppm |
| | | HRMS (ESI): m/z calculated for C₂₉H₃₅O₇⁺ [M + H]⁺: 495.2377; |
| | | found: 495.2342. |
| 3 | C₂₅H₂₆O₅ | ¹H-NMR (500 MHz, (CD₃)₂SO): δ = 10.7 (s, 1 H), 7.99-7.95 |
| (+/− racemic | | (m, 2 H), 7.57-7.51 (m, 3 H), 6.59 (s, 1 H), 6.55 (s, 1 H), 4.61 (d, |
| mixture) | | 1 H), 3.27-3.22 (m, 1 H), 2.63 (dd, 1 H, J = 17.3, 5.3 Hz), 2.31 |
| | | (dd, 1 H, J = 17.3, 12.0 Hz), 1.97-1.92 (m, 1 H), 1.75-1.68 (m, 2 |
| | | H), 1.58-1.49 (m, 2 H), 1.14 (s, 3 H), 1.02 (s, 3 H), 0.79 (s, 3 H) |
| | | ppm |
| | | ¹³C-NMR (125 MHz, (CD₃)₂SO): δ = 175.4 (1 C), 160.0 (1 C), |
| | | 159.0 (1 C), 157.0 (1 C), 153.7 (1 C), 131.2 (1 C), 131.1 (1 C), |
| | | 129.0 (2 C), 125.8 (2 C), 108.0 (1 C), 107.2 (1 C), 106.9 (1 C), |
| | | 93.8 (1 C), 77.0 (1 C), 75.9 (1 C), 46.1 (1 C), 38.0 (1 C), 37.3 (1 |
| | | C), 28.0 (1 C), 27.2 (1 C), 19.5 (1 C), 17.6 (1 C), 14.4 (1 C) ppm. |
| | | HRMS (ESI): m/z calculated for C₂₅H₂₇O₅⁺ [M + H]⁺: 407.1853; |
| | | found: 407.1857. |
| 4 | C₂₇H₃₀O₆ | ¹H-NMR (500 MHz, CD₃CN): δ = 7.94 (dd, 2 H, J = 7.6 Hz), |
| (+) | | 7.55-7.51 (m, 3 H), 6.80 (s, 1 H), 6.51 (s, 1 H), 5.33 (s, 2 H), |
| | | 3.50 (s, 3 H), 3.39-3.33 (m, 1 H), 2.78 (dd, 1 H, J = 17.0, 5.1 |
| | | Hz), 2.76 (s, 1 H), 2.45 (dd, 1 H, J = 17.0, 13.4 Hz), 2.03 (dt, 1 |
| | | H, J = 12.1, 3.0 Hz), 1.82-1.74 (m, 2 H), 1.64-1.58 (m, 2 H), |
| | | 1.21 (s, 3 H), 1.07 (s, 3 H), 0.86 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 78.6 (1 C), 78.0 (1 C), 57.0 (1 C), 46.5 (1 |
| | | C), 39.3 (1 C), 38.6 (1 C), 29.1 (1 C), 27.7 (1 C), 20.2 (1 C), 18.9 |
| | | (1 C), 14.9 (1 C) ppm. |
| | | HRMS (ESI): m/z calculated for C₂₇H₃₁O₆⁺ [M + H]⁺: 451.2115; |
| | | found: 451.2115. |
| 5 | C₂₅H₂₆O₅ | ¹H-NMR (500 MHz, (CD₃)₂SO): δ = 10.7 (s, 1 H), 7.99-7.95 |
| (+) | | (m, 2 H), 7.57-7.51 (m, 3 H), 6.59 (s, 1 H), 6.55 (s, 1 H), 4.61 (d, |
| | | 1 H), 3.27-3.22 (m, 1 H), 2.63 (dd, 1 H, J = 17.3, 5.3 Hz), 2.31 |
| | | (dd, 1 H, J = 17.3, 12.0 Hz), 1.97-1.92 (m, 1 H), 1.75-1.68 (m, 2 |
| | | H), 1.58-1.49 (m, 2 H), 1.14 (s, 3 H), 1.02 (s, 3 H), 0.79 (s, 3 H) |
| | | ppm. |
| | | ¹³C-NMR (125 MHz, (CD₃)₂SO): δ = 175.4 (1 C), 160.0 (1 C), |
| | | 159.0 (1 C), 157.0 (1 C), 153.7 (1 C), 131.2 (1 C), 131.1 (1 C), |
| | | 129.0 (2 C), 125.8 (2 C), 108.0 (1 C), 107.2 (1 C), 106.9 (1 C), |
| | | 93.8 (1 C), 77.0 (1 C), 75.9 (1 C), 46.1 (1 C), 38.0 (1 C), 37.3 (1 |
| | | C), 28.0 (1 C), 27.2 (1 C), 19.5 (1 C), 17.6 (1 C), 14.4 (1 C) ppm. |
| | | HRMS (ESI): m/z calculated for C₂₅H₂₇O₅⁺ [M + H]⁺: 407.1853; |
| | | found: 407.1857. |
| 6 | C₂₇H₃₀O₆ | ¹H-NMR (500 MHz, CD₃CN): δ = 7.94 (dd, 2 H, J = 7.6 Hz), |
| (−) | | 7.55-7.51 (m, 3 H), 6.80 (s, 1 H), 6.51 (s, 1 H), 5.33 (s, 2 H), |
| | | 3.50 (s, 3 H), 3.39-3.33 (m, 1 H), 2.78 (dd, 1 H, J = 17.0, 5.1 |
| | | Hz), 2.76 (s, 1 H), 2.45 (dd, 1 H, J = 17.0, 13.4 Hz), 2.03 (dt, 1 |
| | | H, J = 12.1, 3.0 Hz), 1.82-1.74 (m, 2 H), 1.64-1.58 (m, 2 H), |
| | | 1.21 (s, 3 H), 1.07 (s, 3 H), 0.86 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 78.6 (1 C), 78.0 (1 C), 57.0 (1 C), 46.5 (1 |
| | | C), 39.3 (1 C), 38.6 (1 C), 29.1 (1 C), 27.7 (1 C), 20.2 (1 C), 18.9 |
| | | (1 C), 14.9 (1 C) ppm. |
| | | HRMS (ESI): m/z calculated for C₂₇H₃₁O₆⁺ [M + H]⁺: 451.2115; |
| | | found: 451.2115. |
| 7 | C₂₅H₂₆O₅ | ¹H-NMR (500 MHz, (CD₃)₂SO): δ = 10.7 (s, 1 H), 7.99-7.95 |
| (−) | | (m, 2 H), 7.57-7.51 (m, 3 H), 6.59 (s, 1 H), 6.55 (s, 1 H), 4.61 (d, |
| | | 1 H), 3.27-3.22 (m, 1 H), 2.63 (dd, 1 H, J = 17.3, 5.3 Hz), 2.31 |
| | | (dd, 1 H, J = 17.3, 12.0 Hz), 1.97-1.92 (m, 1 H), 1.75-1.68 (m, 2 |
| | | H), 1.58-1.49 (m, 2 H), 1.14 (s, 3 H), 1.02 (s, 3 H), 0.79 (s, 3 H) |
| | | ppm |
| | | ¹³C-NMR (125 MHz, (CD₃)₂SO): δ = 175.4 (1 C), 160.0 (1 C), |
| | | 159.0 (1 C), 157.0 (1 C), 153.7 (1 C), 131.2 (1 C), 131.1 (1 C), |
| | | 129.0 (2 C), 125.8 (2 C), 108.0 (1 C), 107.2 (1 C), 106.9 (1 C), |
| | | 93.8 (1 C), 77.0 (1 C), 75.9 (1 C), 46.1 (1 C), 38.0 (1 C), 37.3 (1 |
| | | C), 28.0 (1 C), 27.2 (1 C), 19.5 (1 C), 17.6 (1 C), 14.4 (1 C) ppm. |
| | | HRMS (ESI): m/z calculated for C₂₅H₂₇O₅⁺ [M + H]⁺: 407.1853; |
| | | found: 407.1857. |
| 8 | C₂₉H₃₄O₈ | ¹H-NMR (500 MHz, CD₃CN): δ = 7.88 (d, 2 H, J = 8.4 Hz), 7.14 |
| (+) | | (d, 2 H, J = 8.7 Hz), 6.77 (s, 1 H), 6.42 (s, 1 H), 5.32 (s, 2 H), 5.25 |
| | | (s, 2 H), 3.49 (s, 3 H), 3.44 (s, 3 H), 3.38-3.32 (m, 1 H), 3.18 (s, |
| | | 3 H), 2.77 (dd, 1 H, J = 15.8, 6.3 Hz), 2.76 (s, 1 H), 2.44 (dd, 1 H, |
| | | J = 17.4, 14.3 Hz), 2.05-2.00 (m, 1 H), 1.83-1.73 (m, 2 H), 1.65- |
| | | 1.58 (m, 2 H), 1.20 (s, 3 H), 1.07 (s, 3 H), 0.86 (s, 3 H) ppm |
| | | ¹³C-NMR (125 MHz, CD₃CN): δ = 177.3 (1 C), 161.0 (1 C), |
| | | 160.5 (1 C), 159.8 (1 C), 158.6 (1 C), 154.7 (1 C), 128.5 (2 C), |
| | | 126.0 (1 C), 117.3 (2 C), 110.1 (1 C), 109.7 (1 C), 108.3 (1 C), |
| | | 95.3 (1 C), 95.1 (1 C), 94.5 (1 C), 78.4 (1 C), 77.9 (1 C), 56.9 |
| | | (1 C), 56.5 (1 C), 46.4 (1 C), 39.2 (1 C), 38.5 (1 C), 29.0 (1 C), |
| | | 27.6 (1 C), 20.1 (1 C), 18.8 (1 C), 14.8 (1 C) ppm. |
| | | HRMS (ESI): m/z calculated for C₂₉H₃₅O₈⁺ [M + H]⁺: 511.2326; |
| | | found: 511.2326. |
| 9 | C₂₅H₂₆O₆ | ¹H-NMR (500 MHz, MeOD): δ = 7.79 (d, 2 H, ) = 8.0 Hz), 6.92 |
| (+) | | (d, 2 H, J = 8.5 Hz), 6.58-6.48 (m, 2 H), 3.39 (dd, 1 H, J = 11.7, |
| | | 4.2 Hz), 2.78 (dd, 1 H, J = 16.9, 5.0 Hz), 2.43 (dd, 1 H, J = 16.2, |
| | | 14.0 Hz), 2.24-2.16 (m, 1 H), 1.91-1.80 (m, 2 H), 1.73-1.58 (m, |
| | | 2 H), 1.26 (s, 3 H), 1.12 (s, 3 H), 0.91 (s, 3 H) ppm. |
| | | ¹³C-NMR (125 MHz, MeOD): δ = 180.5 (1 C), 163.4 (1 C), |
| | | 162.4 (1 C), 162.2 (1 C), 159.4 (1 C), 155.5 (1 C), 129.0 (2 C), |
| | | 123.5 (1 C), 117.0 (2 C), 109.1 (1 C), 108.4 (1 C), 106.6 (1 C), |
| | | 95.0 (1 C), 79.3 (1 C), 78.7 (1 C), 47.1 (1 C), 39.6 (1 C), 38.5 (1 |
| | | C), 29.1 (1 C), 27.9 (1 C), 19.9 (1 C), 19.1 (1 C), 14.9 (1 C) |
| | | ppm. |
| | | HRMS (ESI): m/z calculated for C₂₅H₂₇O ⁺ [M + H]⁺: 423.1802; |
| | | found: 423.1802. |
| 10 | C₃₁H₃₈O₁₀ | ¹H-NMR (500 MHz, CD₃CN): δ = 8.02 (d, 2 H, J = 8.4 Hz), 7.15 |
| (+) | | (d, 2 H, J = 8.4 Hz), 6.72 (s, 1 H), 5.31 (m, 2 H), 5.25 (s, 2 H), 5.09 |
| | | (q, 2 H, J = 6.4 Hz), 3.48 (s, 3 H), 3.45 (s, 6 H), 3.40-3.35 (m, 1 |
| | | H), 3.18 (s, 3 H), 2.77 (dd, 1 H, J = 15.6, 5.6 Hz), 2.76 (s, 1 H), |
| | | 2.44 (dd, 1 H, J = 16.8, 14.6 Hz), 2.07-2.02 (m, 1 H), 1.83-1.75 |
| | | (m, 2 H), 1.67-1.58 (m, 2 H), 1.21 (s, 3 H), 1.07 (s, 3 H), 0.86 (s, |
| | | 3 H) ppm. |
| | | ¹³C-NMR (125 MHz, CD₃CN): δ = 173.9 (1 C), 160.0 (1 C), |
| | | 159.8 (1 C), 157.8 (1 C), 154.8 (1 C), 153.9 (1 C), 138.6 (1 C), |
| | | 131.2 (2 C), 125.5 (1 C), 116.9 (2 C), 110.3 (1 C), 109.4 (1 C), |
| | | 98.4 (1 C), 95.5 (1 C), 95.2 (1 C), 94.3 (1 C), 78.7 (1 C), 78.0 |
| | | (1 C), 57.9 (1 C), 57.0 (1 C), 56.6 (1 C), 46.5 (1 C), 39.3 (1 C), |
| | | 38.6 (1 C), 29.1 (1 C), 27.7 (1 C), 20.2 (1 C), 18.9 (1 C), 14.9 (1 |
| | | C) ppm. |
| | | HRMS (ESI): m/z calculated for C₃₁H₃₉O₁₀⁺ [M + H]⁺: 571.2538; |
| | | found: 571.2518. |
| 11 | C₂₅H₂₆O₇ | ¹H-NMR (500 MHz, MeOD): δ = 8.07 (d, 2 H, J = 7.4 Hz), 6.90 |
| (+) | | (d, 2 H, J = 8.4 Hz), 6.50 (s, 1 H), 3.40 (dd, 1 H, J = 1.8, 3.7 Hz), |
| | | 2.78 (dd, 1 H, J = 16.9, 5.0 Hz), 2.43 (dd, 1 H, J = 16.2, 14.0 Hz), |
| | | 2.24-2.16 (m, 1 H), 1.91-1.80 (m, 2 H), 1.73-1.58 (m, 2 H), 1.27 |
| | | (s, 3 H), 1.12 (s, 3 H), 0.91 (s, 3 H) ppm. |
| | | ¹³C-NMR (125 MHz, MeOD): δ = 173.7 (1 C), 162.3 (1 C), |
| | | 160.2 (1 C), 158.3 (1 C), 155.0 (1 C), 144.5 (1 C), 130.4 (1 C), |
| | | 130.2 (2 C), 123.9 (1 C), 116.3 (2 C), 108.3 (1 C), 106.4 (1 C), |
| | | 94.6 (1 C), 79.4 (1 C), 78.7 (1 C), 47.2 (1 C), 39.6 (1 C), 38.5 (1 |
| | | C), 29.0 (1 C), 27.9 (1 C), 19.9 (1 C), 18.9 (1 C), 14.9 (1 C) ppm. |
| | | HRMS (ESI): m/z calculated for C₂₅H₂₇O₇⁺ [M + H]⁺: 439.1751; |
| | | found: 439.1751. |
| 12 | C₂₇H₃₀O₆ | ¹H-NMR (500 MHz, CD₃CN): δ = 7.94 (dd, 2 H, J = 7.6 Hz), |
| (+/− racemic | | 7.55-7.51 (m, 3 H), 6.78 (s, 1 H), 6.51 (s, 1 H), 5.33 (q, 2 H, J = |
| mixture) | | 8.6 Hz), 3.49 (s, 3 H), 3.38-3.35 (m, 1 H), 2.80-76 (dd, 2 H, |
| | | J = 17.0, 5.1 Hz), 2.69 (d, 1 H, J = 4.3 Hz), 2.13 (m, 1 H), 2.07- |
| | | 2.01 (m, 1 H), 1.94 (m, 1 H), 1.89-1.83 (m, 1 H), 1.52-1.57 (m, |
| | | 1 H), 1.22 (s, 3 H), 1.01 (3, 3 H), 0.61 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 77.5 (1 C), 75.9 (1 C), 56.9 (1 C), 38.8 |
| | | (1 C), 37.8 (1 C), 32.9 (1 C), 27.7 (1 C), 27.1 (1 C), 25.9 (1 C), |
| | | 22.4 (1 C), 18.7 (1 C) ppm. |
| | | HRMS (ESI): m/z calculated for C₂₇H₃₁O₆⁺ [M + H]⁺: 451.2115; |
| | | found: 451.2061. |
| 13 | C₂₇H₃₀O₆ | ¹H-NMR (500 MHz, CD₃CN): δ = 7.94 (dd, 2 H, J = 7.6 Hz), |
| (+) | | 7.55-7.51 (m, 3 H), 6.78 (s, 1 H), 6.51 (s, 1 H), 5.33 (q, 2 H, J = |
| | | 8.6 Hz), 3.49 (s, 3 H), 3.38-3.35 (m, 1 H), 2.80-76 (dd, 2 H, |
| | | J = 17.0, 5.1 Hz), 2.69 (d, 1 H, J = 4.3 Hz), 2.13 (m, 1 H), 2.07- |
| | | 2.01 (m, 1 H), 1.94 (m, 1 H), 1.89-1.83 (m, 1 H), 1.52-1.57 (m, |
| | | 1 H), 1.22 (s, 3 H), 1.01 (s, 3 H), 0.61 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 77.5 (1 C), 75.9 (1 C), 56.9 (1 C), 38.8 |
| | | (1 C), 37.8 (1 C), 32.9 (1 C), 27.7 (1 C), 27.1 (1 C), 25.9 (1 C), |
| | | 22.4 (1 C), 18.7 (1 C) ppm. |
| | | HRMS (ESI): m/z calculated for C₂₇H₃₁O₆⁺ [M + H]⁺: 451.2115; |
| | | found: 451.2061. |
| 14 | C₂₇H₃₀O₆ | ¹H-NMR (500 MHz, CD₃CN): δ = 7.94 (dd, 2 H, J = 7.6 Hz), |
| (−) | | 7.55-7.51 (m, 3 H), 6.78 (s, 1 H), 6.51 (s, 1 H), 5.33 (q, 2 H, J = |
| | | 8.6 Hz), 3.49 (s, 3 H), 3.38-3.35 (m, 1 H), 2.80-76 (dd, 2 H, |
| | | J = 17.0, 5.1 Hz), 2.69 (d, 1 H, J = 4.3 Hz), 2.13 (m, 1 H), 2.07- |
| | | 2.01 (m, 1 H), 1.94 (m, 1 H), 1.89-1.83 (m, 1 H), 1.52-1.57 (m, |
| | | 1 H), 1.22 (s, 3 H), 1.01 (s, 3 H), 0.61 (s, 3 H) ppm. |
| | | ¹³C-NMR (125 MHz, CD₃CN): δ = 177.3 (1 C), 161.3 (1 C), |
| | | 160.0 (1 C), 158.8 (1 C), 154.8 (1 C), 132.8 (1 C), 132.2 (1 C), |
| | | 130.0 (2 C), 127.0 (2 C), 110.3 (1 C), 109.9 (1 C), 109.5 (1 C), |
| | | 95.4 (1 C), 94.6 (1 C), 77.5 (1 C), 75.9 (1 C), 56.9 (1 C), 38.8 |
| | | (1 C), 37.8 (1 C), 32.9 (1 C), 27.7 (1 C), 27.1 (1 C), 25.9 (1 C), |
| | | 22.4 (1 C), 18.7 (1 C) ppm. |
| | | HRMS (ESI): m/z calculated for C₂₇H₃₁O₆⁺ [M + H]⁺: 451.2115; |
| | | found: 451.2061. |
| 15 | C₃₁H₃₈O₁₀ | ¹H-NMR (500 MHz, CD₃CN): δ = 8.02 (d, 2 H, J = 8.4 Hz), 7.15 |
| (+) | | (d, 2 H, J = 8.4 Hz), 6.72 (s, 1 H), 5.31 (s, 2 H), 5.25 (s, 2 H), 5.09 |
| | | (q, 2 H, J = 6.4 Hz), 3.48 (s, 3 H), 3.45 (s, 6H), 3.38-3.33 (m, 1 |
| | | H), 3.18 (s, 3 H), 2.80-76 (dd, 2 H, J = 17.0, 5.1 Hz), 2.69 (d, 1 |
| | | H, J = 4.3 Hz), 2.13 (m, 1 H), 2.07-2.01 (m, 1 H), 1.94 (m, 1 H), |
| | | 1.89-1.83 (m, 1 H), 1.52-1.57 (m, 1 H), 1.23 (s, 3 H), 1.00 (s, 3 |
| | | H), 0.60 (s, 3 H) ppm. |
| | | HRMS (ESI): m/z calculated for C₃₁H₃₉O₁₀⁺ [M + H]⁺: 571.2538; |
| | | found: 571.2518. |
| 16 | C₃₁H₃₈O₁₀ | ¹H-NMR (500 MHz, CD₃CN): δ = 8.02 (d, 2 H, J = 8.4 Hz), 7.15 |
| (−) | | (d, 2 H, J = 8.4 Hz), 6.72 (s, 1 H), 5.31 (m, 2 H), 5.25 (s, 2 H), 5.09 |
| | | (q, 2 H, J = 6.4 Hz), 3.48 (s, 3 H), 3.45 (s, 6 H), 3.40-3.35 (m, 1 |
| | | H), 3.18 (s, 3 H), 2.77 (dd, 1 H, J = 15.6, 5.6 Hz), 2.76 (s, 1 H), |
| | | 2.44 (dd, 1 H, J = 16.8, 14.6 Hz), 2.07-2.02 (m, 1 H), 1.83-1.75 |
| | | (m, 2 H), 1.67 = 1.58 (m, 2 H), 1.21 (s, 3 H), 1.07 (s, 3 H), 0.86 (s, |
| | | 3 H) ppm. |
| | | ¹³C-NMR (125 MHz, CD₃CN): δ = 173.9 (1 C), 160.0 (1 C), |
| | | 159.8 (1 C), 157.8 (1 C), 154.8 (1 C), 153.9 (1 C), 138.6 (1 C), |
| | | 131.2 (2 C), 125.5 (1 C), 116.9 (2 C), 110.3 (1 C), 109.4 (1 C), |
| | | 98.4 (1 C), 95.5 (1 C), 95.2 (1 C), 94.3 (1 C), 78.7 (1 C), 78.0 |
| | | (1 C), 57.9 (1 C), 57.0 (1 C), 56.6 (1 C), 46.5 (1 C), 39.3 (1 C), |
| | | 38.6 (1 C), 29.1 (1 C), 27.7 (1 C), 20.2 (1 C), 18.9 (1 C), 14.9 (1 |
| | | C) ppm. |
| | | HRMS (ESI): m/z calculated for C₃₁H₃₉O₁₀⁺ [M + H]⁺: 571.2538; |
| | | found: 571.2518. |
| 17 | C₂₅H₂₆O₇ | ¹H-NMR (500 MHz, MeOD): δ = 8.07 (d, 2 H, J = 7.4 Hz), 6.90 |
| (−) | | (d, 2 H, J = 8.4 Hz), 6.50 (s, 1 H), 3.40 (dd, 1 H, J = 1.8, 3.7 Hz), |
| | | 2.78 (dd, 1 H, J = 16.9, 5.0 Hz), 2.43 (dd, 1 H, J = 16.2, 14.0 Hz), |
| | | 2.24-2.16 (m, 1 H), 1.91-1.80 (m, 2 H), 1.73-1.58 (m, 2 H), 1.27 |
| | | (s, 3 H), 1.12 (s, 3 H), 0.91 (s, 3 H) ppm. |
| | | ¹³C-NMR (125 MHz, MeOD): δ = 173.7 (1 C), 162.3 (1 C), |
| | | 160.2 (1 C), 158.3 (1 C), 155.0 (1 C), 144.5 (1 C), 130.4 (1 C), |
| | | 130.2 (2 C), 123.9 (1 C), 116.3 (2 C), 108.3 (1 C), 106.4 (1 C), |
| | | 94.6 (1 C), 79.4 (1 C), 78.7 (1 C), 47.2 (1 C), 39.6 (1 C), 38.5 (1 |
| | | C), 29.0 (1 C), 27.9 (1 C), 19.9 (1 C), 18.9 (1 C), 14.9 (1 C) ppm. |
| | | HRMS (ESI): m/z calculated for C₂₅H₂₇O₇⁺ [M + H]⁺: 439.1751; |
| | | found: 439.1751. |
| 18 | C₂₉H₃₄O₈ | ¹H-NMR (500 MHz, CD₃CN): δ = 7.89 (d, 2 H, J = 8.4 Hz), 7.15 |
| (+) | | (d, 2 H, J = 8.7 Hz), 6.78 (s, 1 H), 6.44 (s, 1 H), 5.34 (q, 2 H, J = |
| | | 7.5 Hz), 5.24 (s, 2 H), 3.49 (s, 3 H), 3.45 (s, 3 H), 3.38-3.33 (m, |
| | | 1 H), 3.18 (s, 3 H), 2.80-76 (dd, 2 H, J = 17.0, 5.1 Hz), 2.69 (d, 1 |
| | | H, J = 4.3 Hz), 2.13 (m, 1 H), 2.07-2.01 (m, 1 H), 1.94 (m, 1 H), |
| | | 1.89-1.83 (m, 1 H), 1.52-1.57 (m, 1 H), 1.23 (s, 3 H), 1.01 (s, 3 |
| | | H), 0.61 (s, 3 H) ppm. |
| | | HRMS (ESI): m/z calculated for C₂₉H₃₅O₈⁺ [M + H]⁺: 511.2326- |
| | | found: 511.2326. |

Method of Preparation of the Compounds of Formula (I) The present invention also relates to a method for preparing a compound of formula (I), as well as the pharmaceutically acceptable salts thereof, its hydrates, its solvates, as well as its tautomeric forms, stereoisomers, mixtures of stereoisomers, pure enantiomers and mixtures, whether or not racemic, as defined in the present invention, comprising at least the following steps:
(i) cyclization of a compound of formula (VII)

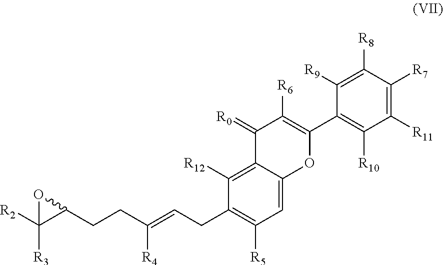

in which
R₀, and R₂ to R₁₂ are as defined according to the present invention, provided that at least
one of R₅ and R₁₂ is a protected hydroxyl group, and the symbol [symbol] represents a single bond joining an asymmetric carbon atom to the group or atom in question, this bond being either in front of or behind the plane, to obtain a compound of formula (I) as defined according to the present invention, and
(ii) Optionally, deprotection of at least one hydroxyl group still protected by a protective group present in said compound obtained in step (i), to obtain a compound of formula (I) as defined according to the present invention.

This reaction corresponds to step (G) as defined in the present invention.

In the present invention, a compound of formula (VII) may be prepared from a compound of formula (VI)

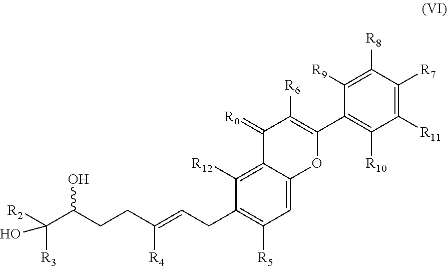

in which
R₀, R₂ to R₁₂ are as defined in the present invention, provided that at least one of R₅ and
R₁₂ is a protected hydroxyl group, and the symbol [symbol] represents a single bond joining an asymmetric carbon atom to the group or atom in question, this bond being either in front of or behind the plane.
or
prepared from a compound of formula (V)

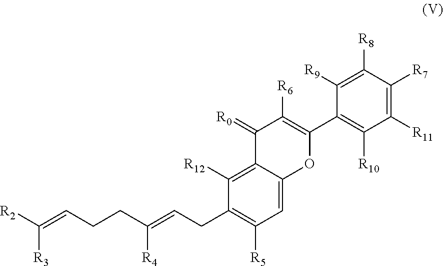

in which
R₀, R₂ to R₁₂ are as defined in the present invention, provided that at least one of R₅ and
R₁₂ is a protected hydroxyl group.

This reaction corresponds to step (F) as defined in the present invention.

According to one variant, when the compound of formula (VII) is prepared from a compound of formula (V), the compound of formula (I) according to the invention is then obtained in the form of a racemic mixture.

According to another variant, when the compound of formula (VII) is prepared from a compound of formula (VI), the compound of formula (I) according to the invention is then obtained in the form of an enantiomer.

In the present invention, a compound of formula (VI) may be prepared by dihydroxylation of a compound of formula (V)

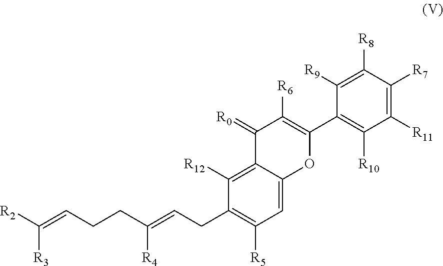

in which
R₀, R₂ to R₁₂ are as defined in the present invention, provided that at least one of R₅ and
R₁₂ is a protected hydroxyl group.

This reaction corresponds to step (E) as defined in the present invention.

In the present invention, a compound of formula (V) may be prepared by geranylation of a compound of formula (IV)

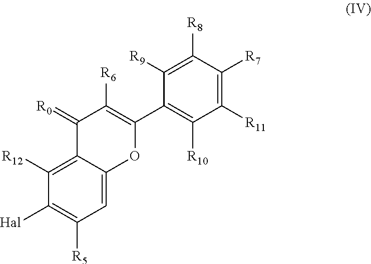

in which
R₀, R₅ to R₁₂ are as defined in the present invention, provided that at least one of R₅ and
R₁₂ is a protected hydroxyl group, and Hal represents a halogen atom, using a compound of formula (VIII)

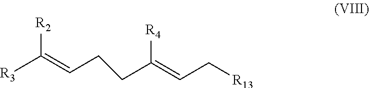

in which R₂, R₃ and R₄ are as defined in the present invention, and R₁₃ represents a group —B(OH)₂ or a group

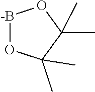

attached by the boron atom to the rest of the molecule.

This reaction corresponds to step (D) as defined in the present invention.

In the present invention, a compound of formula (IV) may be prepared by halogenation, preferably by iodation, of a compound of formula (III)

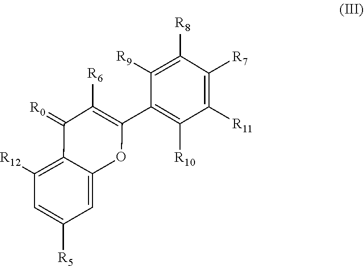

in which R₀, and R₅ to R₁₂ are as defined in the present invention, provided that at least one of R₅ and R₁₂ is a protected hydroxyl group.

This reaction corresponds to step (C) as defined in the present invention.

In the present invention, a compound of formula (III) may be prepared by protection, with the aid of a protective group, of at least one of the two hydroxyl groups present on the benzene nucleus of the benzopyran ring of a compound of formula (II)

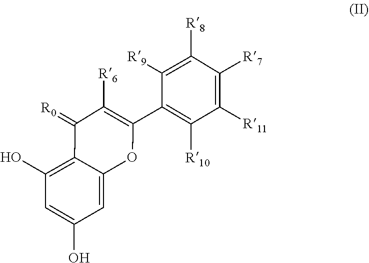

in which
R₀ is as defined according to the present invention; R′₆ to R′₁₁ represent, independently of one another, a hydrogen atom or a hydroxyl group.

This reaction corresponds to step (B) as defined in the present invention.

Thus, according to a particular embodiment, the method for preparing a compound of formula (I) according to the invention comprises at least the following steps: Step (A): Providing a compound of formula (II)

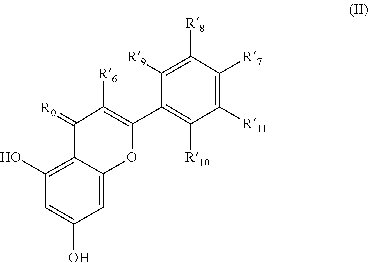

in which
R₀ is as defined according to the present invention;
R′₆ represents a hydrogen atom, a hydroxyl group, a group —O—P(═O)(OH)₂, or a sugar radical, preferably a hydrogen atom or a hydroxyl group,
R′₇ represents a hydrogen atom, a hydroxyl group, a halogen atom, a linear or branched C₁-C₄ alkyl group, a linear or branched C₂-C₄ alkenyl group, a linear or branched C₂-C₄ alkynyl group, a triazolyl group, a group —O—Rb, a group N(Rc)(Rd), a group —C(═O)—N(Re)(Rf), or a group —O—C(═O)—N(Rg)(Rh), preferably a hydrogen atom or a hydroxyl group; Ra, Rb, Rc, Rd, Re, Rf, Rg and Rh representing, independently of one another, a hydrogen atom or a linear or branched C₁-C₄ alkyl group,
R′₈ represents a hydrogen atom or a hydroxyl group,
R′₉ represents a hydrogen atom or a hydroxyl group,
R′₁₀ represents a hydrogen atom or a hydroxyl group, and R′₁₁ represents a hydrogen atom or a hydroxyl group,

Step (B): Protection, by means of a protective group, of at least one of the two hydroxyl groups present on the benzene nucleus of the benzopyran ring of the compound of formula (II) resulting from step (A) in order to obtain a compound of formula (III)

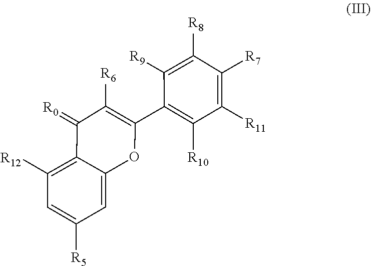

in which R₀, and R₅ to R₁₂ are as defined according to the present invention, provided that at least one of R₅ and R₁₂ is a protected hydroxyl group,

Step (C): Halogenation of the compound of formula (III) obtained in step (B) to obtain a compound of formula (IV)

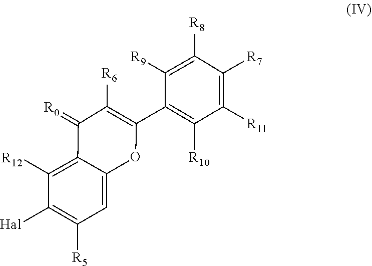

in which R₀, R₅ to R₁₂ are as defined according to the present invention, provided that at least one of R₅ and R₁₂ is a protected hydroxyl group; and Hal represents a halogen atom;

Step (D): Geranylation of the compound of formula (IV) obtained in step (C) using a compound of formula (VIII)

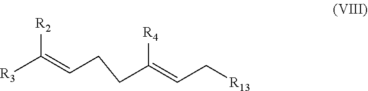

in which R₂, R₃ and R₄ are as defined according to the present invention, and R₁₃ represents a group —B(OH)₂ or a group

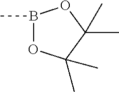

attached by the boron atom to the rest of the molecule, to obtain a compound of formula (V)

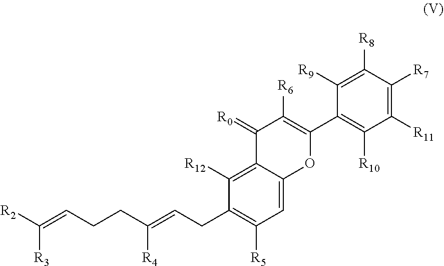

in which R₀, R₂ to R₁₂ are as defined according to the present invention, provided that at least one of R₅ and R₁₂ is a protected hydroxyl group

Step (E): Optionally dihydroxylation of the compound of formula (V) obtained in step (D) to obtain a compound of formula (VI)

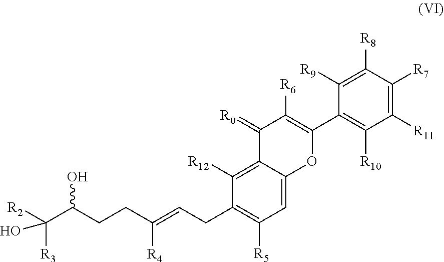

in which R₀, and R₂ to R₁₂ are as defined according to the present invention, provided that at least one of R₅ and R₁₂ is a protected hydroxyl group, and the symbol [symbol] represents a single bond joining an asymmetric carbon atom to the group or atom in question, this bond being either in front of or behind the plane,

Step (F): Formation of the compound of formula (VII) starting from the compound of formula (VI) obtained in step (E) or starting from the compound of formula (V) obtained in step (D)

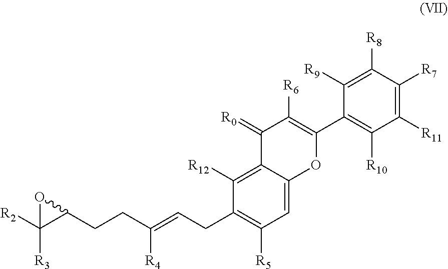

in which R₀, and R₂ to R₁₂ are as defined according to the present invention, provided that at least one of R₅ and R₁₂ is a protected hydroxyl group, and the symbol [symbol] represents a single bond joining an asymmetric carbon atom to the group or atom in question, this bond being either in front of or behind the plane,

### Step (G):

(i) Cyclization of the compound of formula (VII) obtained in step (F) to obtain a compound of formula (I) as defined according to the present invention, and
(ii) Optionally, deprotection of at least one hydroxyl group protected by a protective group present in the compound obtained in step (i) to obtain a compound of formula (I) as defined according to the present invention.

The following two reaction schemes illustrate the method of preparation according to the invention.

The first reaction scheme labeled scheme 1 comprises the steps (B), (C), (D), (F), (G) (i) or (G) (ii). In other words, this scheme illustrates the method of preparation according to the invention when the latter does not comprise the dihydroxylation step (E).

The second reaction scheme labeled scheme 2 comprises steps (B), (C), (D), (E), (F), (G) (i) or (G) (ii). In other words, this scheme illustrates the method of preparation according to the invention when the latter does comprise the dihydroxylation step (E).

In these two schemes, it is to be understood that to obtain a compound of formula (IV) as defined in the invention starting from a compound of formula (III) as defined in the invention, it is necessary to carry out either step (C) alone, or step (C) followed by protection of hydroxyl group(s) (OH).

Moreover, in these two schemes, it is to be understood that step (G) comprises either only step (G)(i), or step (G)(i) followed by step (G)(ii)):
substep (G) (i) corresponds to cyclization of a compound of formula (VII) to obtain a compound of formula (I) according to the invention, and
substep (G) (ii), therefore optional, corresponds to deprotection of hydroxyl group(s), with a view to obtaining a deprotected compound of formula (I) according to the invention, when said compound is required.

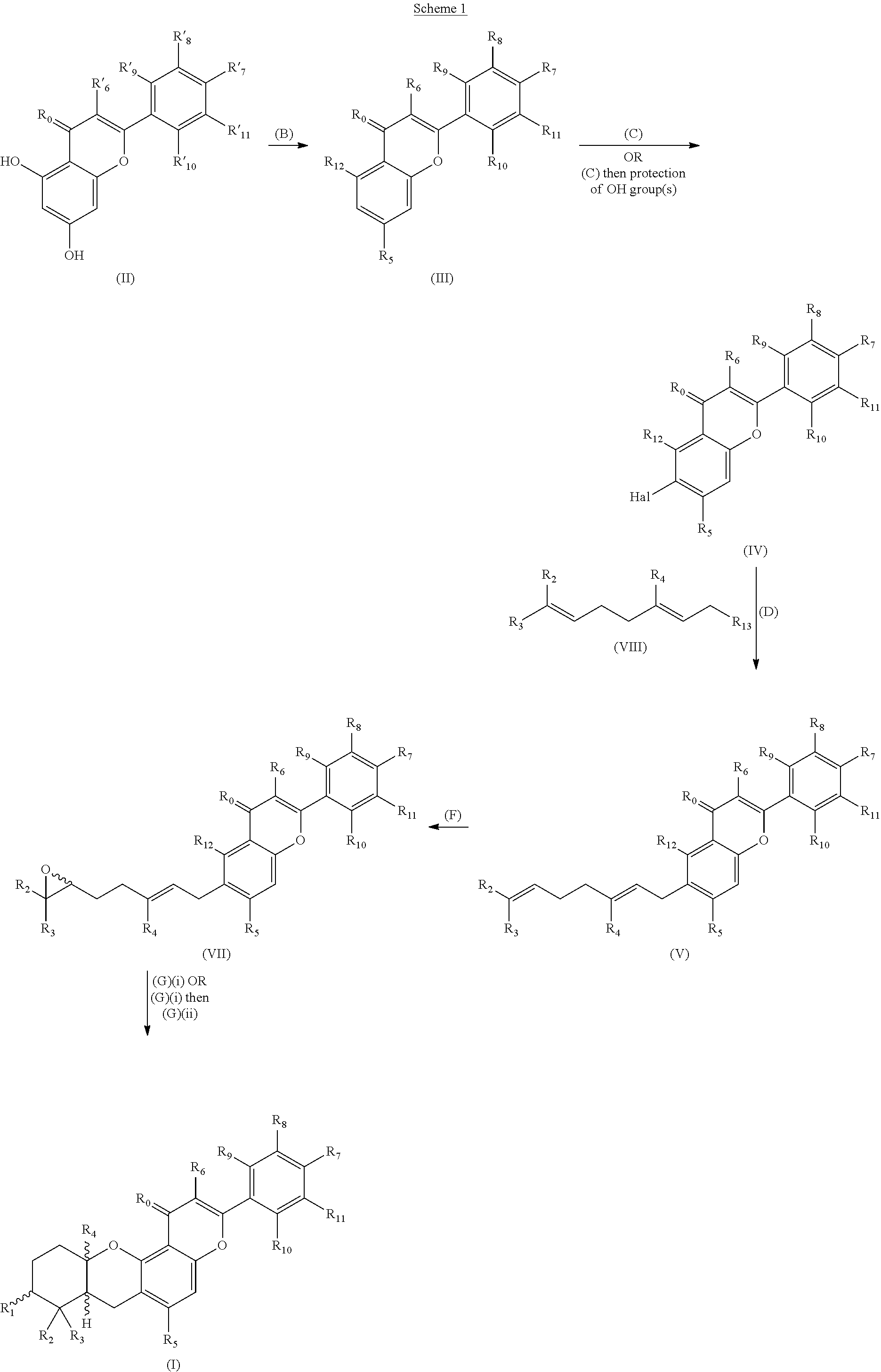

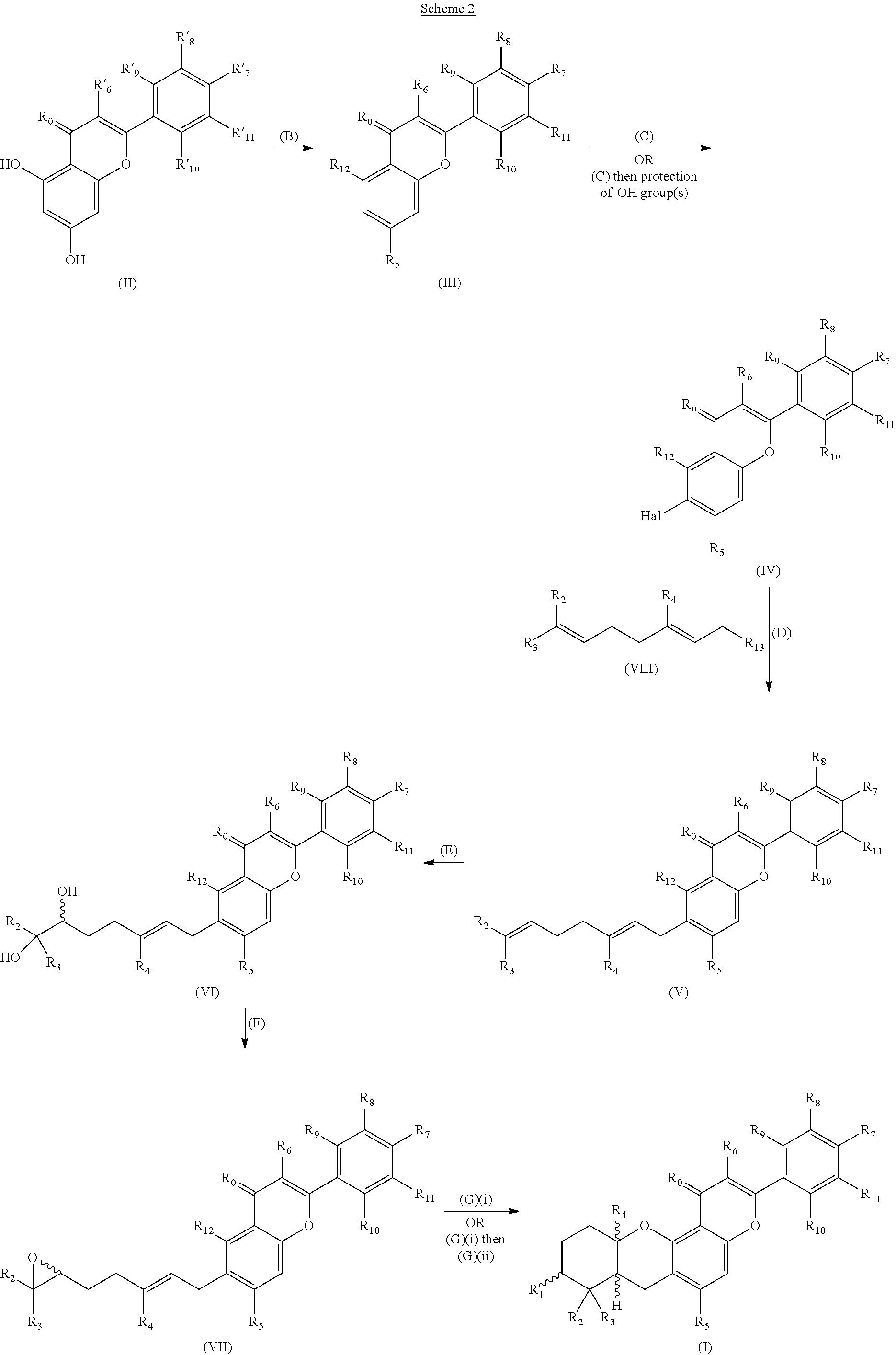

### Step (A)

The starting products (compounds of formula (II)) used for this step are commercially available natural compounds or can easily be synthesized by methods familiar to a person skilled in the art. Their synthesis is described for example in application CN109180628.

### Step B

Step (B) corresponds to protection of hydroxyl function(s) present in compounds according to formula (II) in order to obtain a compound of formula (III).

The reaction of protection of at least one of the two hydroxyl groups present on the benzene nucleus of the benzopyran ring may be carried out by any conventional method known by a person skilled in the art using a protective group in order to obtain a protected hydroxyl group.

According to one variant, only one of the two hydroxyl groups present on the benzene nucleus of the benzopyran ring is protected at the end of step (B).

According to another variant, only the two hydroxyl groups present on the benzene nucleus of the benzopyran ring are protected at the end of step (B).

According to yet another variant, in addition to one of the two hydroxyl groups present on the benzene nucleus of the benzopyran ring, all the other hydroxyl groups present in the compound of formula (II) are protected at the end of step (B).

According to yet another variant, all the hydroxyl groups present in the compound of formula (II) are protected at the end of step (B).

According to yet another variant, only one of the two hydroxyl groups present on the benzene nucleus of the benzopyran ring and at least one of the other hydroxyl groups optionally present in the compound of formula (II) are protected at the end of step (B).

According to yet another variant, the two hydroxyl groups present on the benzene nucleus of the benzopyran ring and at least one of the other hydroxyl groups optionally present in the compound of formula (II) are protected at the end of step (B).

According to a particular embodiment, this reaction may be carried out in the presence of a base selected from potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably K₂CO₃.

According to another particular embodiment, this reaction may be carried out in the presence of a solvent selected from acetonitrile, an acetonitrile/DMSO (acetonitrile/dimethylsulfoxide) mixture, acetone, THF (tetrahydrofuran), DMF (N,N-dimethylformamide), DMM (dimethoxymethane), DCM (dichloromethane), dioxane, MTBE (methyl tert-butyl ether), diethyl ether, toluene, and mixtures thereof, preferably acetonitrile and an acetonitrile/DMSO mixture, more preferably acetonitrile.

According to another particular embodiment, mixing of the compound of formula (II), the solvent, and the base as defined in the invention may be carried out in an inert atmosphere, for example under argon, and then bringing to a temperature between 0° C. and 50° C.; preferably at 40° C., 45° C. or at 0° C. for a time between 2 h and 24 h, preferably 30 min, 1 h, 3 h or 4 h.

According to another particular embodiment, the reagent supplying the protective group is selected from the methyl halomethyl ethers, preferably methyl chloromethyl ether (CH₃—O—CH₂—Cl or abbreviated to MOMCl), methyl iodomethyl ether, methyl bromomethyl ether, and mixtures thereof, more preferably methyl chloromethyl ether.

According to one embodiment, the reagent supplying the protective group may be added to the mixture formed from the compound of formula (II), the solvent, and the base as defined in the invention for a time between 30 min and 48 h, preferably for 1 h, 3 h, 4 h, 6 h or 24 h, at a temperature between 0° C. and 50° C., preferably at 40° C., 45° C. or at 0° C.

According to a particular embodiment, this protection reaction may be carried out in the presence of a base selected from potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably K₂CO₃, a solvent selected from acetonitrile, an acetonitrile/DMSO (acetonitrile/dimethylsulfoxide) mixture, acetone, THF (tetrahydrofuran), DMF (N,N-dimethylformamide), DMM (dimethoxymethane), DCM (dichloromethane), dioxane, MTBE (methyl tert-butyl ether), diethyl ether, toluene, and mixtures thereof, preferably acetonitrile and an acetonitrile/DMSO mixture, more preferably acetonitrile, and a reagent supplying the protective group selected from the methyl halomethyl ethers, preferably methyl chloromethyl ether (CH₃—O—CH₂—Cl or abbreviated to MOMCl), methyl iodomethyl ether, methyl bromomethyl ether, and mixtures thereof, more preferably methyl chloromethyl ether.

According to a particularly preferred embodiment, this protection reaction may be carried out in the presence of a base, potassium carbonate K₂CO₃, a solvent, acetonitrile, or an acetonitrile/DMSO mixture, and a reagent supplying a protective group, namely methyl chloromethyl ether.

Advantageously, the product of formula (III) obtained according to the invention may be purified by any conventional method known by a person skilled in the art, for example by silica gel column chromatography using a heptane/ethyl acetate mixture in proportions that a person skilled in the art will be able to adapt.

### Step (C)

Step (C) corresponds to halogenation of a compound of formula (III) in order to obtain a compound of formula (IV).

The halogenation reaction may be carried out by any conventional method known by a person skilled in the art.

According to one variant, this step is an iodation.

According to another variant, this step is a chlorination.

According to another variant, this step is a bromination.

According to another variant, this step is a fluorination.

According to a particular embodiment, this reaction may be carried out in the presence of a base selected from sodium hydrogen carbonate (NaHCO₃), potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably sodium hydrogen carbonate (NaHCO₃).

According to another particular embodiment, this reaction may be carried out in the presence of a reagent supplying the halogen atom selected from benzyltrimethylammonium dichloroiodate (BTMA.ICl₂), N-iodosuccinimide (NIS), N-bromosuccinimide (NBS), N-chlorosuccinimide (NCS), diiodine (I₂), dibromine (Br₂), dichlorine (Cl₂), potassium iodide (KI) and mixtures thereof, preferably benzyltrimethylammonium dichloroiodate (BTMA.ICl₂).

According to a particular embodiment, this reaction may be carried out in the presence of a solvent selected from a dichloromethane/methanol (DCM/MeOH) mixture in a volume ratio from 10:1 to 1:10, preferably in a volume ratio of 5:2 or of 2:1, acetone, acetonitrile (ACN), dioxane, MTBE (methyl tert-butyl ether), diethyl ether, THF (tetrahydrofuran), DMF (N,N-dimethylformamide), DMM (dimethoxymethane), ethanol (EtOH), propanol, butanol, toluene, and mixtures thereof, preferably a dichloromethane/methanol mixture.

According to a particular embodiment, this reaction is an iodation reaction, which may be carried out in the presence of a base selected from sodium hydrogen carbonate (NaHCO₃), potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably sodium hydrogen carbonate (NaHCO₃), a reagent supplying the halogen atom in particular the iodine atom selected from benzyltrimethylammonium dichloroiodate (BTMA.ICl₂), N-iodosuccinimide (NIS), N-bromosuccinimide (NBS), N-chlorosuccinimide (NCS), diiodine (I₂), dibromine (Br₂), dichlorine (Cl₂), potassium iodide (KI) and mixtures thereof, preferably benzyltrimethylammonium dichloroiodate (BTMA.ICl₂), and a solvent selected from a dichloromethane/methanol (DCM/MeOH) mixture in a volume ratio from 10:1 to 1:10, preferably in a volume ratio of 5:2 or of 2:1, acetone, acetonitrile (ACN), dioxane, MTBE (methyl tert-butyl ether), diethyl ether, THF (tetrahydrofuran), DMF (N,N-dimethylformamide), DMM (dimethoxymethane), ethanol (EtOH), propanol, butanol, toluene, and mixtures thereof, preferably a dichloromethane/methanol mixture.

According to a particularly preferred embodiment, this halogenation reaction is a reaction of iodation, which may be carried out in the presence of a base, NaHCO₃, a solvent, namely a dichloromethane/methanol mixture, and a reagent, namely benzyltrimethylammonium dichloroiodate.

According to another particular embodiment, mixing of the compound of formula (III), the solvent, the base and the compound (or reagent) supplying the halogen atom as defined in the invention may be carried out at a temperature between 10° C. and 40° C.; preferably at room temperature, for a time between 30 min and 24 h, preferably 1 h, 3 h or 8 h.

Advantageously, this step is carried out in the dark.

Advantageously, the product of formula (IV) obtained according to the invention may be purified by any conventional method known by a person skilled in the art, for example by silica gel column chromatography using a heptane/ethyl acetate mixture in proportions that a person skilled in the art will be able to adapt.

According to a particular embodiment, a step of protection of hydroxyl group(s) remaining in the structure of the compound of formula (IV) in question, used conventionally by a person skilled in the art, may be carried out, if necessary, between step (C) and step (D) as defined in the present invention. This protection may be carried out for example with the same reagents and in the same conditions as in step (B) as defined in the present invention.

### Step (D)

Step (D) corresponds to geranylation of a compound of formula (IV) using a compound of formula (VIII) in order to obtain a compound of formula (V).

The reaction of geranylation may be carried out by any conventional method known by a person skilled in the art.

The compound of formula (VIII) suitable for the invention therefore constitutes a reagent supplying the group of the geranyl type to the compound of formula (IV). According to a particular embodiment, the compound of formula (VIII) is of formula (VIII-a) given hereunder:

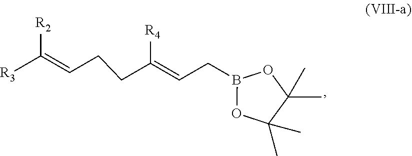

R₄ are as defined in the present invention; preferably they each represent a methyl group.

According to another particular embodiment, the compound of formula (VIII) is of formula (VIII-b) given hereunder:

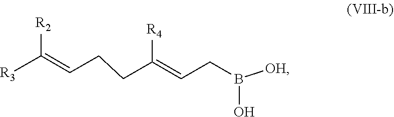

are as defined in the present invention; preferably they each represent a methyl group.

According to a particularly preferred embodiment, the compound of formula (VIII) is selected from geranyl boronic ester (belonging to formula VIII-a in which R₂, R₃ and R₄ each represent a methyl group), geranyl boronic acid (belonging to formula VIII-b in which R₂, R₃ and R₄ each represent a methyl group) and mixtures thereof, preferably geranyl boronic ester.

According to a particular embodiment, this reaction may be carried out in the presence of a base selected from sodium hydroxide (NaOH), sodium hydrogen carbonate (NaHCO₃), potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably sodium hydroxide.

According to another particular embodiment, this reaction may be carried out in the presence of a solvent selected from a teltrahydrofuran/water (THF/H₂O) mixture in a volume ratio from 10:1 to 1:10, preferably of 2:1, acetone, acetonitrile (ACN), dioxane, MTBE (methyl tert-butyl ether), diethyl ether, DMF (N,N-dimethylformamide), DMM (dimethoxymethane), ethanol (EtOH), propanol, butanol, toluene and mixtures thereof, preferably a tetrahydrofuran/water mixture.

Advantageously, the solvent or the mixture of solvents may be degassed.

According to another particular embodiment, this reaction may be carried out in the presence of a catalyst selected from palladium-tetrakis(triphenylphosphine) (Pd(PPh₃)₄), [1,1′-bis(diphenylphosphino)ferrocene]palladium(II) dichloride (PdCl₂(dppf)), bis(triphenylphosphine)palladium chloride (Pd(PPh₃)₂Cl₂), palladium(II) acetate (Pd(OAc)₂), dichlorobis(tri-o-tolylphosphine)palladium(II) (PdCl₂[P(o-Tol)₃]₂), [1,1′-bis(di-tert-butylphosphino)ferrocene]dichloropalladium(II) (PdCl₂(dtbpf)), tris(dibenzylideneacetone)dipalladium(0) (Pd₂dba₃), and mixtures thereof, preferably palladium-tetrakis(triphenylphosphine).

According to another particular embodiment, this reaction may be carried out in the presence of a base selected from sodium hydroxide (NaOH), sodium hydrogen carbonate (NaHCO₃), potassium carbonate (K₂CO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably sodium hydroxide, a reagent supplying the group of the geranyl type selected from geranyl boronic ester, geranyl boronic acid and mixtures thereof, preferably geranyl boronic ester, a catalyst selected from palladium-tetrakis(triphenylphosphine) (Pd(PPh₃)₄), [1,1-bis(diphenylphosphino)ferrocene]palladium(II) dichloride (PdCl₂(dppf)), bis(triphenylphosphine)palladium chloride (Pd(PPh₃)₂Cl₂), palladium(II) acetate (Pd(OAc)₂), dichlorobis(tri-o-tolylphosphine)palladium(II) (PdCl₂[P(o-Tol)₃]₂), [1,1′-bis(di-tert-butylphosphino)ferrocene]dichloropalladium(II) (PdCl₂(dtbpf)), tris(dibenzylideneacetone)dipalladium(0) (Pd₂dba₃), and mixtures thereof, preferably palladium-tetrakis(triphenylphosphine), and a solvent selected from a tetrahydrofuran/water (THF/H₂O) mixture in a volume ratio from 10:1 to 1:10, preferably of 2:1, acetone, acetonitrile (ACN), dioxane, MTBE (methyl tert-butyl ether), diethyl ether, DMF (N,N-dimethylformamide), DMM (dimethoxymethane), ethanol (EtOH), propanol, butanol, toluene and mixtures thereof, preferably a tetrahydrofuran/water mixture.

According to a particularly preferred embodiment, this reaction may be carried out in the presence of a base of sodium hydroxide, a solvent, namely a tetrahydrofuran/water mixture, a catalyst, which is palladium-tetrakis(triphenylphosphine), and a reagent, which is geranyl boronic ester.

According to another particular embodiment, mixing of the compound of formula (IV), the solvent, the base, the catalyst and the compound (or reagent) supplying the group of the geranyl type as defined in the invention may be carried out at a temperature between 70° C. and 100° C., preferably at 90° C. or at 100° C. for a time between 1 h and 8 h, preferably 2 h, 4 h or 5 h.

Advantageously, heating is carried out in a microwave.

Advantageously, the product of formula (V) obtained according to the invention may be purified by any conventional method known by a person skilled in the art, for example by silica gel column chromatography using a heptane/ethyl acetate mixture in proportions that a person skilled in the art will be able to adapt.

According to one variant, at the end of this step (D), when a compound of formula (I) according to the invention is required in the form of a racemic mixture, the compound of formula (V) thus obtained is submitted to step (F) directly, i.e. without undergoing step (E). This applies in particular to the specific compounds of formula (I) according to the invention numbered 1, 2, 3 and 12.

According to another variant, at the end of this step (D), when a compound of formula (I) according to the invention is required in the form of a pure enantiomer, the compound of formula (V) thus obtained is submitted to step (E). This applies in particular to the specific compounds of formula (I) according to the invention numbered 4 to 11 and 13 to 18.

### Step (E)

Step (E) is optional and corresponds to dihydroxylation of a compound of formula (V) in order to obtain a compound of formula (VI).

As stated above, a compound of formula (V) according to the invention is submitted to this reaction if the compound of formula (I) according to the invention is required in the form of a pure enantiomer.

The reaction of dihydroxylation may be carried out by any conventional method known by a person skilled in the art. For example, it may be Sharpless asymmetric dihydroxylation. According to a particular embodiment, this reaction may be carried out in the presence of a base selected from potassium carbonate (K₂CO₃), sodium hydrogen carbonate (NaHCO₃), sodium hydride (NaH), potassium hydroxide (KOH), sodium hydroxide (NaOH), cesium carbonate (Cs₂CO₃), N,N-diisopropylethylamine (DIPEA), pyridine and mixtures thereof, preferably potassium carbonate.

According to another particular embodiment, this reaction may be carried out in the presence of an osmium catalyst selected from osmium tetroxide (OsO₄), potassium osmate (K₂[OsO₂(OH)₄]), and mixtures thereof, preferably osmium tetroxide.

According to another particular embodiment, this reaction may be carried out in the presence of a reoxidant of the osmium catalyst selected from potassium ferricyanide (K₃[Fe(CN)₆]), sodium chlorite (NaClO₂), N-methylmorpholine oxide (NMO) and mixtures thereof, preferably potassium ferricyanide.

According to another particular embodiment, this reaction may be carried out in the presence of a solvent selected from a tert-butanol/water (t-BuOH/H₂O) mixture in a volume ratio from 3:1 to 1:3, preferably in a volume ratio of 1:2, propanol, butanol, and mixtures thereof, preferably a tert-butanol/water mixture.

According to another particular embodiment, when an oxidizing agent comprising an osmium atom selected from osmium tetroxide (OsO₄), potassium osmate (K₂[OsO₂(OH)₄]), and mixtures thereof, preferably osmium tetroxide, is used, this reaction may be carried out in the presence of a chiral ligand selected from hydroquinine 1,4-phthalazinediyl diether ((DHQ)₂PHAL), hydroquinidine 1,4-phthalazinediyl diether ((DHQD)₂PHAL), the derivatives of these ligands, and mixtures thereof, preferably hydroquinine 1,4-phthalazinediyl diether and hydroquinidine 1,4-phthalazinediyl diether.

According to one variant, this reaction is carried out advantageously in the presence of an additive, namely mesylamine (CH₃SO₂NH₂).

According to another variant, this reaction is not carried out in the presence of an additive, namely mesylamine (CH₃SO₂NH₂).

According to another particular embodiment, this reaction may be carried out in the presence of a base selected from potassium carbonate (K₂CO₃), sodium hydrogen carbonate (NaHCO₃), sodium hydride (NaH), potassium hydroxide (KOH), sodium hydroxide (NaOH), cesium carbonate (Cs₂CO₃), N,N-diisopropylethylamine (DIPEA), pyridine, and mixtures thereof, preferably potassium carbonate, an osmium catalyst selected from osmium tetroxide (OsO₄), potassium osmate (K₂[OsO₂(OH)₄]), and mixtures thereof, preferably osmium tetroxide, a reoxidant of the osmium catalyst selected from potassium ferricyanide (K₃[Fe(CN)₆]), sodium chlorite (NaClO₂), N-methylmorpholine oxide (NMO), and mixtures thereof, preferably potassium ferricyanide, a chiral ligand selected from hydroquinine 1,4-phthalazinediyl diether ((DHQ)₂PHAL), hydroquinidine 1,4-phthalazinediyl diether ((DHQD)₂PHAL), the derivatives of these ligands, and mixtures thereof, preferably hydroquinine 1,4-phthalazinediyl diether and hydroquinidine 1,4-phthalazinediyl diether, a solvent selected from a tert-butanol/water (t-BuOH/H₂O) mixture in a volume ratio from 3:1 to 1:3, preferably in a volume ratio of 1:2, propanol, butanol, and mixtures thereof, preferably a tert-butanol/water mixture, and optionally an additive, namely CH₃SO₂NH₂.

According to a particularly preferred embodiment, this reaction may be carried out in the presence of a base, namely potassium carbonate, a solvent, which is a tert-butanol/water mixture, an osmium catalyst, namely osmium tetroxide, a reoxidant, which is potassium ferricyanide, a chiral ligand, which is hydroquinine 1,4-phthalazinediyl diether or alternatively hydroquinidine 1,4-phthalazinediyl diether, and an additive, namely methanesulfonamide.

According to another particular embodiment, mixing of the compound of formula (V), the solvent, the base, the oxidizing agent in a catalytic amount, the reoxidant of the chiral ligand, and optionally of the additive, as defined in the invention, may be carried out at a temperature between −5° C. and 30° C.; preferably at 0° C. for a time between 2 h and 24 h; preferably 2 h or 16 h.

Advantageously, the product of formula (VI) obtained according to the invention may be purified by any conventional method known by a person skilled in the art, for example by silica gel column chromatography using a heptane/ethyl acetate mixture in proportions that a person skilled in the art will be able to adapt.

### Step (F)

Step (F) corresponds to formation of a compound of formula (VII) either starting from a compound of formula (VI) obtained at the end of step (E), or starting from a compound of formula (V) obtained at the end of step (D).

This reaction makes it possible to generate an epoxide function in the structure of the compounds of formula (V) (from a —C═C— double bond) or (VI) (from 2 hydroxyl groups, each borne by a carbon atom, the two carbon atoms being adjacent to one another, namely —C(OH)—C(OH)—) as defined in the present invention. It may be carried out by any conventional method known by a person skilled in the art.

Step (F) Starting from the Compound of Formula (V)

According to a particular embodiment, this reaction may be carried out in the presence of a solvent selected from dichloromethane, toluene, chloroform, 1,2-dichloroethane, benzene, tert-butyl alcohol, ethyl acetate (EtOAc), methanol, water, and mixtures thereof, preferably dichloromethane.

Advantageously, once the solvent has been added to the compound of formula (V), the mixture may be cooled to a temperature between −10° C. and 0° C., preferably to 0° C.

According to a particular embodiment, this reaction may be carried out in the presence of an agent conventionally used for reactions of epoxidation on the alkenes selected from metachloroperbenzoic acid (m-CPBA), tert-butyl hydroperoxide (t-BuOOH), hydrogen peroxide (H₂02), perbenzoic acid, oxone, and mixtures thereof, preferably m-CPBA.

Advantageously, once the agent used for reactions of epoxidation on the alkenes has been added, the mixture may be stirred for a time between 30 min and 24 h; preferably 2.5 h, at a temperature between −10° C. and 0° C., preferably at 0° C.

According to another particular embodiment, this reaction may be carried out in the presence of a solvent selected from dichloromethane, toluene, chloroform, 1,2-dichloroethane, benzene, tert-butyl alcohol, ethyl acetate (EtOAc), methanol, water, and mixtures thereof, preferably dichloromethane, and an agent used for reactions of epoxidation on the alkenes selected from metachloroperbenzoic acid (m-CPBA), tert-butyl hydroperoxide (t-BuOOH), hydrogen peroxide (H₂02), perbenzoic acid, oxone, and mixtures thereof, preferably m-CPBA.

According to a particularly preferred embodiment, this reaction may be carried out in the presence of a solvent, namely dichloromethane, and an agent used for reactions of epoxidation on the alkenes, namely m-CPBA.

Step (F) Starting from the Compound of Formula (VI)

According to a particular embodiment, this reaction comprises formation of an intermediate comprising a mesylate function in its structure.

According to a particular embodiment, this reaction may be carried out in the presence of a first solvent selected from anhydrous dichloromethane, dioxane, MTBE (methyl tert-butyl ether), diethyl ether, THF (tetrahydrofiuran), DMM (dimethoxymethane), toluene, chloroform, 1,2-dichloroethane, benzene, and mixtures thereof, preferably anhydrous dichloromethane.

According to another particular embodiment, this reaction may be carried out in the presence of a first base selected from triethylamine, 1,8-diazabicyclo[5.4.0]undec-7-ene (DBU), pyridine, sodium hydroxide (NaOH), and mixtures thereof, preferably triethylamine.

Advantageously, the mixing of the mixture formed from the compound of formula (VI), the first base and the first solvent may be carried out at a temperature between −5° C. and 30° C., preferably at 0° C., for a time from 15 min to 24 h, preferably for 30 min or 1 h.

Advantageously, the reaction is carried out in an inert atmosphere, for example under argon.

According to another particular embodiment, this reaction may be carried out in the presence of an agent capable of forming a mesylate group, namely methanesulfonyl chloride (MsCl).

According to another particular embodiment, this reaction may be carried out in the presence of a second base selected from 1,8-diazabicyclo[5.4.0]undec-7-ene (DBU), triethylamine, pyridine, and mixtures thereof, preferably DBU. This second base is added to the reaction mixture formed from at least the first base, a compound of formula (VI), the first solvent and the agent capable of forming a mesylate group as defined in the present invention.

According to another embodiment, this reaction may be carried out in the presence of a second solvent selected from anhydrous dichloromethane, dioxane, MTBE (methyl tert-butyl ether), diethyl ether, THF (tetrahydrofuran), DMM (dimethoxymethane), toluene, chloroform, 1,2-dichloroethane, benzene, and mixtures thereof, preferably anhydrous dichloromethane.

Advantageously, the first solvent and the second solvent are identical.

Advantageously, once the second base has been added to the reaction mixture, the temperature of the reaction mixture may be between −5° C. and 40° C., preferably may be at room temperature or at 0° C., for a time between 15 min and 24 h, preferably 2 h or 17 h.

According to another particular embodiment, the first base is selected from triethylamine, 1,8-diazabicyclo[5.4.0]undec-7-ene (DBU), pyridine, sodium hydroxide (NaOH), and mixtures thereof, preferably triethylamine, the first solvent is selected from anhydrous dichloromethane, dioxane, MTBE (methyl tert-butyl ether), diethyl ether, THF (tetrahydrofuran), DMM (dimethoxymethane), toluene, chloroform, 1,2-dichloroethane, benzene, and mixtures thereof, preferably anhydrous dichloromethane, the agent capable of forming a mesylate is methanesulfonyl chloride, the second base is selected from DBU, triethylamine, pyridine, and mixtures thereof, preferably DBU, and the second solvent is selected from anhydrous dichloromethane, dioxane, MTBE (methyl tert-butyl ether), diethyl ether, THF (tetrahydrofuran), DMM (dimethoxymethane), toluene, chloroform, 1,2-dichloroethane, benzene, and mixtures thereof, preferably anhydrous dichloromethane.

According to a particularly preferred embodiment, this reaction may be carried out in the presence of a first base, namely triethylamine, a first solvent and a second solvent, which are anhydrous dichloromethane, an agent capable of forming a mesylate, which is methanesulfonyl chloride, and a second base, which is DBU.

Advantageously, the product of formula (VII) obtained according to the invention may be purified by any conventional method known by a person skilled in the art, for example by silica gel column chromatography using a heptane/ethyl acetate mixture in proportions that a person skilled in the art will be able to adapt.

### Step (G)

Step (G) corresponds to cyclization proper of a compound of formula (VII) (substep i)) followed optionally by deprotection of hydroxyl group(s) (substep ii)), with a view to obtaining a compound of formula (I) according to the invention.

According to a variant, this step may comprise only substep i) as defined in the present invention, which therefore corresponds to cyclization proper.

According to another variant, this step may comprise substep i) as defined in the present invention, followed by substep ii) as defined in the present invention, if deprotection of hydroxyl group(s) is required.

### Substep i)

According to a particular embodiment, the cyclization reaction may be carried out in the presence of a solvent selected from anhydrous dichloromethane, toluene, chloroform, 1,2-dichloroethane, benzene, DMM (dimethoxymethane) and mixtures thereof, preferably anhydrous dichloromethane.

Advantageously, the reaction is carried out in an inert atmosphere, for example under argon.

According to a particular embodiment, the cyclization reaction may be carried out in the presence of a reagent selected from Me₂AlCl (dimethyl aluminum chloride) in hexane, BF₃·OEt₂ (ethyl diether boron trifluoride), Yb(OTf)₃·H₂O (ytterbium triflate monohydrate), TMSCl (trimethyl silyl chloride), ZnCl₂ (zinc chloride), SnCl₄ (tin tetrachloride), montmorillonite K10, and mixtures thereof, preferably from Me₂AlCl in hexane, and BF₃·OEt₂.

According to a particular embodiment, mixing of a compound of formula (VII) according to the invention, the solvent and the aforementioned reagent may be carried out at a temperature between −78° C. and 20° C., preferably at −78° C., for a time between 10 min and 3 h, preferably 30 min, 1 h or 2.5 h.

According to a particular embodiment, this reaction i) may be carried out in the presence of a solvent selected from anhydrous dichloromethane, toluene, chloroform, 1,2-dichloroethane, benzene, DMM (dimethoxymethane) and mixtures thereof, preferably anhydrous dichloromethane, and a reagent selected from Me₂AlCl (dimethyl aluminum chloride) in hexane, BF₃·OEt₂ (ethyl diether boron trifluoride), Yb(OTf)₃·H₂O (ytterbium 20 triflate monohydrate), TMSCl (trimethyl silyl chloride), ZnCl₂ (zinc chloride), SnCl₄ (tin tetrachloride), montmorillonite K10, and mixtures thereof, preferably from Me₂AlCl in hexane, and BF₃·OEt₂.

According to a particularly preferred embodiment, this reaction i) may be carried out in the presence of a solvent, which is anhydrous dichloromethane, and a reagent, which is Me₂AlCl in hexane or BF₃·OEt₂.

Advantageously, the product of formula (I) according to the invention obtained at the end of substep i) may be purified by any conventional method known by a person skilled in the art, for example by silica gel column chromatography using a heptane/ethyl acetate mixture in proportions that a person skilled in the art will be able to adapt.

### Substep ii)

According to a particular embodiment, the deprotection reaction may be carried out in the presence of a solvent selected from acetonitrile, an anhydrous methanol/THF mixture in a volume ratio from 10:1 to 1:10, preferably in a volume ratio of 2:1, methanol, acetone, acetonitrile (ACN), dioxane, MTBE (methyl tert-butyl ether), diethyl ether, THF (tetrahydrofuran), DMF (N,N-dimethylformamide), DMM (dimethoxymethane), ethanol (EtOH), propanol, butanol, toluene, chloroform, 1,2-dichloroethane, benzene, tert-butyl alcohol, ethyl acetate (EtOAc), and mixtures thereof, preferably from acetonitrile, anhydrous methanol/THF mixture in a volume ratio from 10:1 to 1:10, and methanol.

According to a particular embodiment, the deprotection reaction may be carried out in the presence of an acid selected from hydrochloric acid, bismuth chloride, silica, sodium hydrogenate, zinc bromide, paratoluenesulfonic acid, bromodimethylborane, trifluoroacetic acid, acetic acid, sulfuric acid, zinc tetrachloride (ZnCl₄), camphosulfonic acid, ytterbium chloride (YbCl₂), and mixtures thereof, preferably from hydrochloric acid.

According to another particular embodiment, instead of the aforementioned acid, an ion exchange resin, in particular that marketed under the name Dowex® 50wx8 200-400 by the Dow Chemical Company, may be used.

According to a particular embodiment, reaction ii) as defined in the invention may be carried out at a temperature between 15° C. and 50° C., preferably at 35° C. or 38° C., and for a time between 12 h and 5 days, preferably 22 h, 40 h, 2 days or 4 days.

According to a particular embodiment, this reaction ii) may be carried out in the presence of a solvent selected from acetonitrile, an anhydrous methanol/THF mixture in a volume ratio from 10:1 to 1:10, preferably in a volume ratio of 2:1, methanol, acetone, acetonitrile (ACN), dioxane, MTBE (methyl tert-butyl ether), diethyl ether, THF (tetrahydrofuran), DMF (N,N-dimethylformamide), DMM (dimethoxymethane), ethanol (EtOH), propanol, butanol, toluene, chloroform, 1,2-dichloroethane, benzene, tert-butyl alcohol, ethyl acetate (EtOAc), and mixtures thereof, preferably from acetonitrile, anhydrous methanol/THF mixture in a volume ratio from 10:1 to 1:10, and methanol, an acid selected from hydrochloric acid, bismuth chloride, silica, sodium hydrogenate, zinc bromide, paratoluenesulfonic acid, bromodimethylborane, trifluoroacetic acid, acetic acid, sulfuric acid, zinc tetrachloride (ZnCl₄), camphosulfonic acid, ytterbium chloride (YbCl₂), and mixtures thereof, preferably from hydrochloric acid, or an ion exchange resin, preferably Dowex® 50wx8 200-400.

According to a particularly preferred embodiment, this reaction ii) may be carried out in the presence of a solvent, which is acetonitrile, anhydrous methanol/THF mixture in a volume ratio from 10:1 to 1:10, preferably in a volume ratio of 2:1, or methanol, an acid, which is hydrochloric acid, or in place of the acid, an ion exchange resin.

Advantageously, the product of formula (I) according to the invention obtained at the end of substep ii) may be purified by any conventional method known by a person skilled in the art, for example by silica gel column chromatography using a heptane/ethyl acetate mixture in proportions that a person skilled in the art will be able to adapt.

According to one variant, when a compound of formula (I) is required in the form of a racemic mixture, the method according to the invention comprises at least steps (A), (B), (C), (D), (F) and (G). In other words, the method does not comprise step (E).

According to another variant, when a compound of formula (I) is desired in the form of a pure enantiomer, the method according to the invention comprises at least steps (A), (B), (C), (D), (E), (F) and (G).

### Intermediates According to the Invention

According to another aspect, the present invention relates to new intermediates of formulas (IV), (V), (VI) and (VII) as such, their pharmaceutically acceptable salts, their hydrates, their solvates, as well as their tautomeric forms, stereoisomers, mixtures of stereoisomers, pure enantiomers and mixtures, whether or not racemic, as defined in the present application and detailed hereunder:

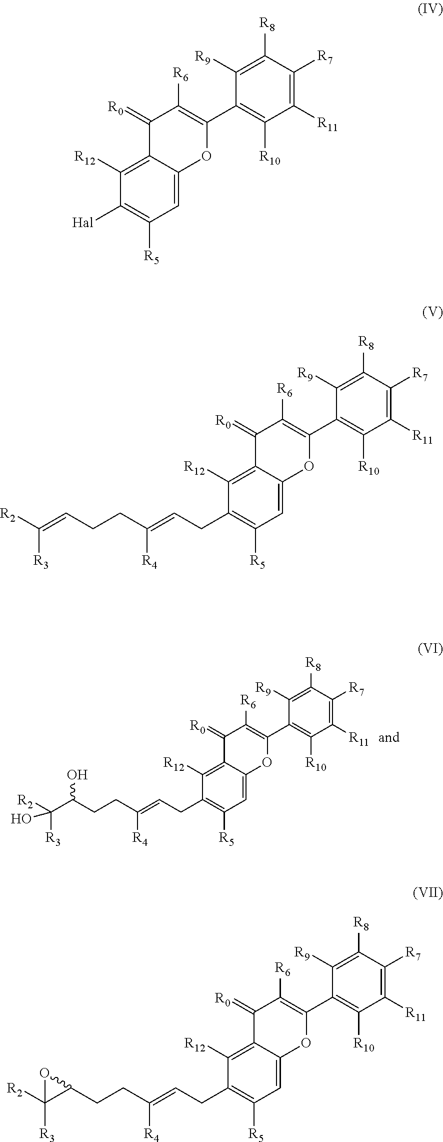

in which R₀, R₂, R₃, R₄, Hal, R₅, R₆, R₇, R₈, R₉, R₁₀, R₁₁ and R₁₂ are as defined according to the present invention, provided that at least one of R₅ and R₁₂ is a protected hydroxyl group, Hal is a halogen atom, and the symbol [symbol] represents a single bond joining an asymmetric carbon atom to the group or atom in question, this bond being either in front of or behind the plane, and provided that:
for the compounds of formula (IV):
   when R₇ is a benzyloxy group, R₅ is different from a methoxymethoxy group, a benzyloxy group, or a hydroxyl group,
   when R₇ is a methoxy group, R₅ is different from a methoxy group, a beta-glucosyloxy group or a hexaacetyl-beta-rutinosyloxy group,
   when R₇ is a hydrogen atom, R₅ is different from a methoxy group or a methoxymethoxy group, and
   when R₇ is an ethoxy group, R₅ is different from an ethoxy group; and provided that
for the compounds of formula (V):
   when R₇ is a methoxymethoxy group, R₆ is different from a hydrogen atom, and
   when R₇ is a methoxy group, R₆ is different from a benzoyloxy group.

According to a particular embodiment, in the compound of formula (IV), R₀ represents an oxygen atom; Hal is a halogen atom; R₅ represents a protected hydroxyl group; R₆ represents a hydrogen atom or a protected hydroxyl group; R₇ represents a protected hydroxyl group; R₈, R₉, R₁₀, and R₁₁ each represent a hydrogen atom; and R₁₂ represents a hydroxyl group.

According to a particularly preferred embodiment, in the compound of formula (IV), R₀ represents an oxygen atom, Hal is an iodine atom, R₅ represents a group —O—CH₂—O—CH₃, R₆ represents a hydrogen atom or a group —O—CH₂—O—CH₃, R₇ represents a group —O—CH₂—O—CH₃, R₈, R₉, R₁₀, and R₁₁ each represent a hydrogen atom, and R₁₂ represents a hydroxyl group.

According to a more preferred embodiment, the new compound of formula (IV) as defined in the invention is selected from the following compounds shown in Tables 3 and 4, characterized by NMR (nuclear magnetic resonance):

TABLE 3

| Number of the intermediate and | |
|---|---|
| chemical name | Structure of the compound |
| IV-1 5-hydroxy-6-iodo-3,7-bis(methoxymethoxy)- 2-(4-(methoxymethoxy)phenyl)-4H-chromen- 4-one | 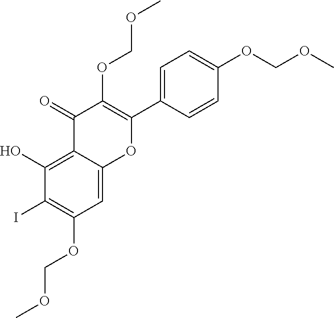 [SMILES: COCOc1ccc(-c2oc3cc(OCOC)c(I)c(O)c3c(=O)c2OCOC)cc1] |
| IV-2 5-hydroxy-6-iodo-7-(methoxymethoxy)-2-(4- (methoxymethoxy)phenyl)-4H-chromen-4-one | 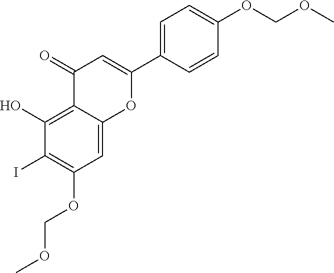 [SMILES: COCOc1ccc(-c2cc(=O)c3c(O)c(I)c(OCOC)cc3o2)cc1] |

TABLE 4

| Number of the intermediate | Characterization (NMR) |
|---|---|
| IV-1 | ¹H-NMR (500 MHz, (CD₃)CN): δ = 13.64 (s, |
| | 1 H), 8.08 (d, 2 H, J = 9.0 Hz), 7.19 (d, 2 H), |
| | 6.84 (s, 1 H), 5.36 (s, 2 H), 5.27 (s, 2 H), 5.14 |
| | (s, 2 H), 3.50 (s, 3 H), 3.45 (s, 1 H), 3.21 (s, 3 |
| | H) ppm. |
| IV-2 | ¹H-NMR (500 MHz, (CD₃)CN): δ = 13.96 (s, |
| | 1 H), 7.96 (d, 2 H, J = 8.5 Hz), 7.18 (d, 2 H), |
| | 6.89 (s, 1 H), 6.75 (s, 1 H), 5.37 (s, 2 H), 5.27 |
| | (s, 2 H), 3.50 (s, 3 H), 3.45 (s, 3 H) ppm. |

According to a particular embodiment, in the compound of formula (V), R₀ represents an oxygen atom; R₂ represents a linear or branched C₁-C₄ alkyl group; R₃ represents a linear or branched C₁-C₄ alkyl group; R₄ represents a linear or branched C₁-C₄ alkyl group; R₅ represents a protected hydroxyl group; R₃ represents a hydrogen atom or a protected hydroxyl group; R₇ represents a hydrogen atom or a protected hydroxyl group; R₈, R₉, R₁₀, and R₁₁ each represent a hydrogen atom; and R₁₂ represents a hydroxyl group or a protected hydroxyl group.

According to a particularly preferred embodiment, in the compound of formula (V), R₀ represents an oxygen atom; R₂ represents a methyl group; R₃ represents a methyl group; R₄ represents a methyl group; R₅ represents a group —O—CH₂—O—CH₃; R₆ represents a hydrogen atom or a group —O—CH₂—O—CH₃; R₇ represents a hydrogen atom or a group —O—CH₂—O—CH₃; R₈, R₉, R₁₀, and R₁₁ each represent a hydrogen atom; and R₁₂ represents a hydroxyl group or a group —O—CH₂—O—CH₃.

According to another more preferred embodiment, the new compound of formula (V) as defined in the invention is selected from the following compounds shown in Tables 5 and 6, characterized by NMR (nuclear magnetic resonance) and/or by mass spectrometry:

TABLE 5

| Number of the intermediate and | |
|---|---|
| chemical name | Structure of the compound |
| V-1 (E)-6-(3,7-dimethylocta-2,6-dien-1-yl)- 5,7-bis(methoxymethoxy)-2-phenyl-4H- chromen-4-one | 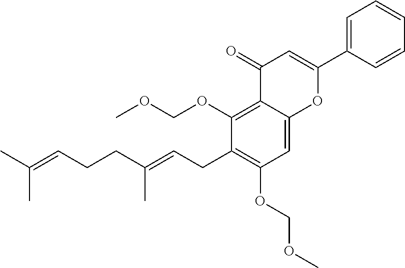 [SMILES: COCOc1cc2oc(-c3ccccc3)cc(=O)c2c(OCOC)c1C/C=C(\C)CCC=C(C)C] |
| V-2 stereoisomer (Z)-6-(3,7-dimethylocta-2,6-dien-1-yl)- 5,7-bis(methoxymethoxy)-2-phenyl-4H- chromen-4-one | 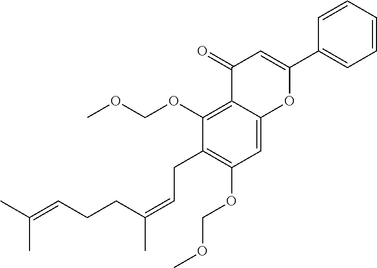 [SMILES: COCOc1cc2oc(-c3ccccc3)cc(=O)c2c(OCOC)c1C/C=C(/C)CCC=C(C)C] |
| V-3 (E)-6-(3,7-dimethylocta-2,6-dien-1-yl)-5- hydroxy-7-(methoxymethoxy)-2-phenyl- 4H-chromen-4-one | 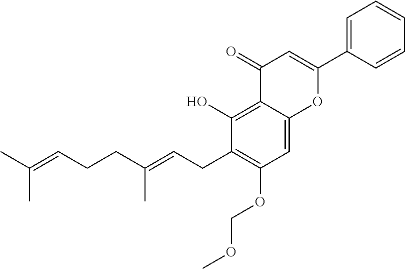 [SMILES: COCOc1cc2oc(-c3ccccc3)cc(=O)c2c(O)c1C/C=C(\C)CCC=C(C)C] |
| V-4 stereoisomer (Z)-6-(3,7-dimethylocta-2,6-dien-1-yl)-5- hydroxy-7-(methoxymethoxy)-2-phenyl- 4H-chromen-4-one | 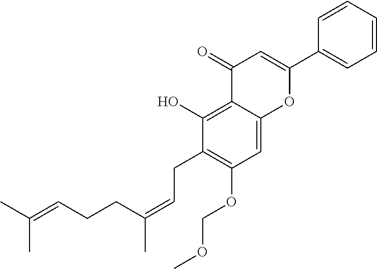 [SMILES: COCOc1cc2oc(-c3ccccc3)cc(=O)c2c(O)c1C/C=C(/C)CCC=C(C)C] |
| V-5 (E)-6-(3,7-dimethylocta-2,6-dien-1-yl)-5- hydroxy-3,7-bis(methoxymethoxy)-2-(4- (methoxymethoxy)phenyl)-4H-chromen- 4-one | 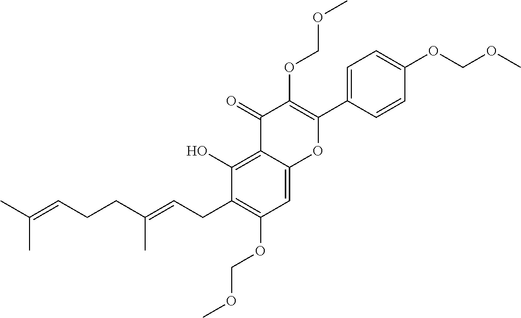 [SMILES: COCOc1ccc(-c2oc3cc(OCOC)c(C/C=C(\C)CCC=C(C)C)c(O)c3c(=O)c2OCOC)cc1] |
| V-6 stereoisomer (Z)-6-(3,7-dimethylocta-2,6-dien-1-yl)-5- hydroxy-3,7-bis(methoxymethoxy)-2-(4- (methoxymethoxy)phenyl)-4H-chromen- 4-one | 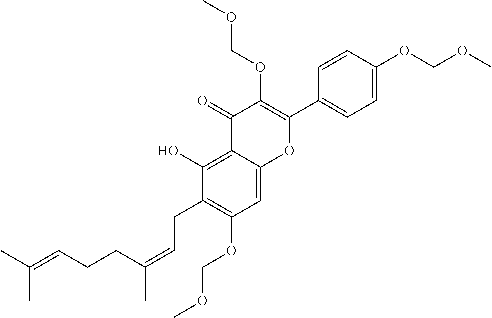 [SMILES: COCOc1ccc(-c2oc3cc(OCOC)c(C/C=C(/C)CCC=C(C)C)c(O)c3c(=O)c2OCOC)cc1] |

TABLE 6

| Number of the | |
|---|---|
| intermediate | Characterization (NMR and MS) |
| V-1 | ¹H-NMR (500 MHz, CD₃CN): δ = 7.96 (dd, 2 H, J = 7.9, 1.6 Hz), |
| | 7.57-7.52 (m, 3 H), 7.08 (s, 1 H), 6.62 (s, 1 H), 5.33 (s, 2 H), 5.22 (t, |
| | 1 H, J = 7.5 Hz), 5.09 (s, 2 H), 5.06 (t, 1 H, J = 7.5 Hz), 3.55 (s, 3 H), |
| | 3.49 (d, 2 H, J = 6.8 Hz), 3.46 (s, 3 H), 2.26 (t, 1 H, J = 8.4 Hz), 2.14 |
| | (t, 1 H, J = 8.6 Hz), 2.04 (t, 1 H, J = 7.3 Hz), 1.98 (d, 1 H, J = 8.6 |
| | Hz), 1.80 (s, 3 H), 1.61 (s, 3 H), 1.55 (s, 3 H) ppm. |
| | ¹³C-NMR (125 MHz, CD₃CN): δ = 183.9 (1 C), 165.2 (1 C), 161.8 |
| | (1 C), 159.5 (1 C), 157.0 (1 C), 136.4 (1 C), 133.0 (1 C), 132.4 (2 |
| | C), 130.2 (2 C), 127.5 (2 C), 127.3 (1 C), 122.9 (1 C), 114.3 (1 C), |
| | 106.8 (1 C), 106.6 (1 C), 95.3 (1 C), 93.7 (1 C), 57.0 (1 C), 40.5 (1 C), |
| | 27.5 (1 C), 25.9 (1 C), 22.1 (1 C), 17.8 (1 C), 16.4 (1 C) ppm. |
| | HRMS (ESI): m/z calculated for C₂₉H₃₅O₆⁺ [M + H]⁺: 479.2428; |
| | found: 479.2416 |
| V-2 | ¹H-NMR (500 MHz, CD₃CN): δ = 7.96 (dd, 2 H, J = 7.9, 1.6 Hz), |
| stereoisomer | 7.57-7.52 (m, 3 H), 7.08 (s, 1 H), 6.62 (s, 1 H), 5.33 (s, 2 H), 5.22 (t, |
| | 1 H, J = 7.5 Hz), 5.09 (s, 2 H), 5.06 (t, 1 H, J = 7.5 Hz), 3.55 (s, 3 H), |
| | 3.49 (d, 2 H, J = 6.8 Hz), 3.46 (s, 3 H), 2.26 (t, 1 H, J = 8.4 Hz), 2.14 |
| | (t, 1 H, J = 8.6 Hz), 2.04 (t, 1 H, J = 7.3 Hz), 1.98 (d, 1 H, J = 8.6 Hz), |
| | 1.69 (s, 3 H), 1.67 (s, 3 H), 1.64 (s, 3 H) ppm. |
| | HRMS (ESI): m/z calculated for C₂₉H₃₅O₆⁺ [M + H]⁺: 479.2428; found: |
| | 479.2416. |
| V-3 | ¹H-NMR (500 MHz, CD₃CN): δ = 13.04 (s, 1 H), 8.00 (d, 2 H, J = 7.9 |
| | Hz), 7.59 (d, 1 H, J = 6.6 Hz), 7.57 (dd, 2 H, J = 13.6 Hz), 6.82 (s, |
| | 1 H), 6.76 (s, 1 H), 5.32 (s, 2 H), 5.23 (t, 1 H, J = 8.4 Hz), 5.06 (t, 1 |
| | H, J = 8.4 Hz), 3.47 (s, 3 H), 3.36 (d, 2 H, J = 7.6 Hz), 2.26 (t, 1 H, |
| | J = 8.4 Hz), 2.14 (t, 1 H, J = 8.6 Hz), 2.04 (t, 1 H, J = 7.3 Hz), 1.98 |
| | (d, 1 H, J = 8.6 Hz), 1.80 (s, 3 H), 1.61 (s, 3 H), 1.55 (s, 3 H) ppm. |
| | ¹³C-NMR (125 MHz, CD₃CN): δ = 183.9 (1 C), 165.2 (1 C), 161.8 |
| | (1 C), 159.5 (1 C), 157.0 (1 C), 136.4 (1 C), 133.0 (1 C), 132.4 (2 |
| | C), 130.2 (2 C), 127.5 (2 C), 127.3 (1 C), 122.9 (1 C), 114.3 (1 C), |
| | 106.8 (1 C), 106.6 (1 C), 95.3 (1 C), 93.7 (1 C), 57.0 (1 C), 40.5 (1 C), |
| | 27.5 (1 C), 25.9 (1 C), 22.1 (1 C), 17.8 (1 C), 16.4 (1 C) ppm. |
| | HRMS (ESI): m/z calculated for C₂₇H₃₁O₅⁺ [M + H]⁺: 435.2166; |
| | found: 435.2165 |
| V-4 | ¹H-NMR (500 MHz, CD₃CN): δ = 13.04 (s, 1 H), 8.00 (d, 2 H, J = |
| stereoisomer | 7.9 Hz), 7.59 (d, 1 H, J = 6.6 Hz), 7.57 (dd, 2 H, J = 13.6 Hz), 6.82 |
| | (s, 1 H), 6.76 (s, 1 H), 5.32 (s, 2 H), 5.23 (t, 1 H, J = 8.4 Hz), 5.06 |
| | (t, 1 H, J = 8.4 Hz), 3.47 (s, 3 H), 3.36 (d, 2 H, J = 7.6 Hz), 2.26 (t, |
| | 1 H, J = 8.4 Hz), 2.14 (t, 1 H, J = 8.6 Hz), 2.04 (t, 1 H, J = 7.3 Hz), |
| | 1.98 (d, 1 H, J = 8.6 Hz), 1.69 (s, 3 H), 1.67 (s, 3 H), 1.64 (s, 3 H) ppm. |
| | HRMS (ESI): m/z calculated for C₂₇H₃₁O₅⁺ [M + H]⁺: 435.2166; found: |
| | 435.2165 |
| V-5 | ¹H-NMR (500 MHz, CD₃CN): δ = 12.80 (s, 1 H), 8.05 (d, 2 H, J = 9.6 |
| | Hz), 7.16 (dd, 2 H, J = 8.4 Hz), 6.73 (s, 1 H), 5.29 (s, 2 H), 5.26 (s, 2 |
| | H), 5.21 (t, 1 H J = 8.4 Hz), 5.13 (s, 2 H), 5.05 (t, 1 H, J = 6.6 Hz), |
| | 3.45 (s, 6 H), 3.35 (d, 2 H, J = 8.4 Hz), 3.20 (s, 3 H), 2.27-2.22 (m, 1 |
| | H), 2.15-2.02 (m, 2 H), 1.99-1.94 (m, 1 H), 1.79 (s, 3 H), 1.64 (s, 3 |
| | H), 1.55 (s, 3 H) ppm. |
| | ¹³C-NMR (125 MHz, CD₃CN): δ = 179.8 (1 C), 161.7 (1 C), 160.4 |
| | (1 C), 159.2 (1 C), 157.7 (1 C), 156.0 (1 C), 136.7 (1 C), 136.4 (1 |
| | C), 132.2 (1 C), 131.6 (2 C), 125.3 (1 C), 125.0 (1 C), 122.9 (1 C), |
| | 117.0 (2 C), 113.8 (1 C), 107.0 (1 C), 98.9 (1 C), 95.3 (1 C), 95.2 (1 |
| | C), 93.4 (1 C), 58.1 (1 C), 57.0 (1 C), 56.7 (1 C), 40.5 (1 C), 27.5 (1 |
| | C), 25.9 (1 C), 22.2 (1 C), 17.8 (1 C), 16.4 (1 C) ppm. |
| | HRMS (ESI): m/z calculated for C₃₁H₃₉O₉⁺ [M + H]⁺: 555.2589; found: |
| | 555.2580 |
| V-6 | ¹H-NMR (500 MHz, CD₃CN): δ = 12.82 (s, 1H), 8.05 (d, 2 H, J = 9.6 |
| stereoisomer | Hz), 7.16 (dd, 2 H, J = 8.4 Hz), 6.73 (s, 1 H), 5.29 (s, 2 H), 5.26 (s, 2 |
| | H), 5.21 (t, 1 H J = 8.4 Hz), 5.13 (s, 2 H), 5.13 (t, 1 H, J = 6.6 Hz), |
| | 3.45 (s, 6 H), 3.35 (d, 2 H, J = 8.4 Hz), 3.20 (s, 3 H), 2.27-2.22 (m, 1 |
| | H), 2.15-2.02 (m, 2 H), 1.99-1.94 (m, 1 H), 1.69 (s, 3 H), 1.66 (s, 3 |
| | H), 1.64 (s, 3 H) ppm. |
| | ¹³C-NMR (125 MHz, CD₃CN): δ = 179.8 (1 C), 161.7 (1 C), 160.4 |
| | (1 C), 159.2 (1 C), 157.7 (1 C), 156.0 (1 C), 136.8 (1 C), 136.4 (1 |
| | C), 132.4 (1 C), 131.6 (2 C), 125.5 (1 C), 125.0 (1 C), 123.7 (1 C), |
| | 117.0 (2 C), 114.9 (1 C), 107.0 (1 C), 98.9 (1 C), 95.3 (1 C), 95.2 (1 |
| | C), 93.4 (1 C), 58.1 (1 C), 57.0 (1 C), 56.7 (1 C), 32.7 (1 C), 27.5 (1 |
| | C), 26.0 (1 C), 23.6 (1 C), 22.1 (1 C), 17.9 (1 C) ppm |
| | HRMS (ESI): m/z calculated for C₃₁H₃₉O₉⁺ [M + H]⁺: 555.2589; found: |
| | 555.2580. |

According to a particular embodiment, in the compound of formula (VI), R₀ represents an oxygen atom; R₂ represents a linear or branched C₁-C₄ alkyl group; R₃ represents a linear or branched C₁-C₄ alkyl group; R₄ represents a linear or branched C₁-C₄ alkyl group; R₅ represents a protected hydroxyl group; R₄ represents a hydrogen atom or a protected hydroxyl group; R₇ represents a hydrogen atom or a protected hydroxyl group; R₅, R₉, R₁₀, and R₁₁ each represent a hydrogen atom; and R₁₂ represents a hydroxyl group or a protected hydroxyl group.

According to a particularly preferred embodiment, in the compound of formula (VI), R₀ represents an oxygen atom; R₂ represents a methyl group; R₃ represents a methyl group; R₄ represents a methyl group; R₅ represents a group —O—CH₂—O—CH₃; R₆ represents a hydrogen atom or a group —O—CH₂—O—CH₃; R₇ represents a hydrogen atom or a group —O—CH₂—O—CH₃; R₈, R₉, R₁₀, and R₁₁ each represent a hydrogen atom; and R₁₂ represents a hydroxyl group or a group —O—CH₂—O—CH₃.

According to another more preferred embodiment, the new compound of formula (VI) as defined in the invention is selected from the following compounds shown in Tables 7 and 8, characterized by NMR (nuclear magnetic resonance) and/or by mass spectrometry:

TABLE 7

| Number of the intermediate and | |
|---|---|
| chemical name | Structure of the compound |
| VI-1 (S,E)-6-(6,7-dihydroxy-3,7- dimethyloct-2-en-1-yl)-5-hydroxy-7- (methoxymethoxy)-2-phenyl-4H- chromen-4-one | 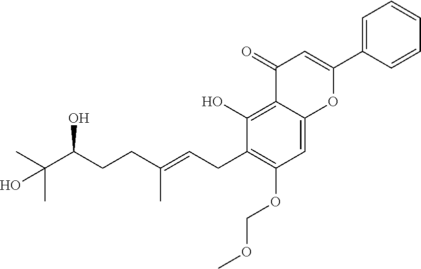 [SMILES: COCOc1cc2oc(-c3ccccc3)cc(=O)c2c(O)c1C/C=C(\C)CC[C@H](O)C(C)(C)O] |
| VI-2 stereoisomer (S,Z)-6-(6,7-dihydroxy-3,7- dimethyloct-2-en-1-yl)-5-hydroxy-7- (methoxymethoxy)-2-phenyl-4H- chromen-4-one | 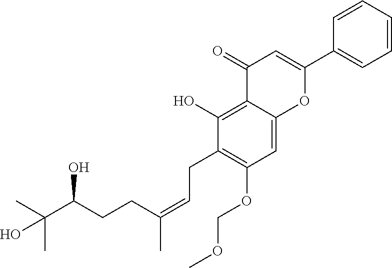 [SMILES: COCOc1cc2oc(-c3ccccc3)cc(=O)c2c(O)c1C/C=C(/C)CC[C@H](O)C(C)(C)O] |
| VI-3 (R,E)-6-(6,7-dihydroxy-3,7- dimethyloct-2-en-1-yl)-5-hydroxy-7- (methoxymethoxy)-2-phenyl-4H- chromen-4-one | 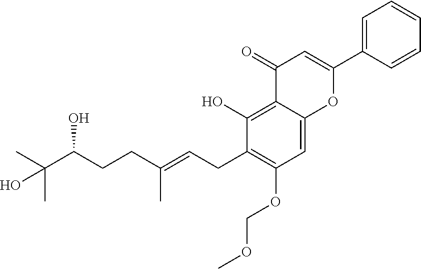 [SMILES: COCOc1cc2oc(-c3ccccc3)cc(=O)c2c(O)c1C/C=C(\C)CC[C@@H](O)C(C)(C)O] |
| VI-4 stereoisomer (R,Z)-6-(6,7-dihydroxy-3,7- dimethyloct-2-en-1-yl)-5-hydroxy-7- (methoxymethoxy)-2-phenyl-4H- chromen-4-one | 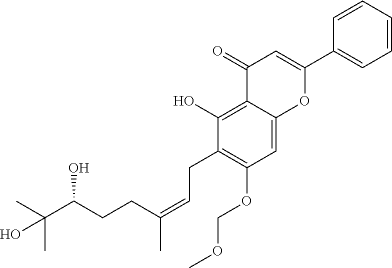 [SMILES: COCOc1cc2oc(-c3ccccc3)cc(=O)c2c(O)c1C/C=C(/C)CC[C@@H](O)C(C)(C)O] |
| VI-5 (S,E)-6-(6,7-dihydroxy-3,7- dimethyloct-2-en-1-yl)-5-hydroxy-3,7- bis(methoxymethoxy)-2-(4- (methoxymethoxy)phenyl)-4H- chromen-4-one | 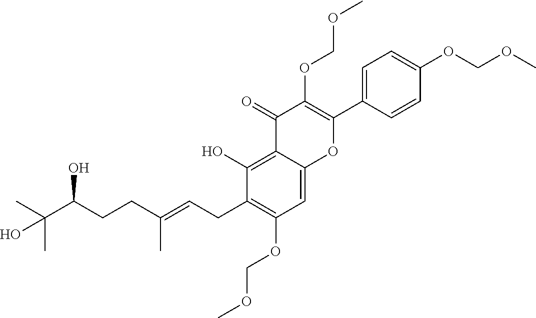 [SMILES: COCOc1ccc(-c2oc3cc(OCOC)c(C/C=C(\C)CC[C@H](O)C(C)(C)O)c(O)c3c(=O)c2OCOC)cc1] |
| VI-6 stereoisomer (S,Z)-6-(6,7-dihydroxy-3,7- dimethyloct-2-en-1-yl)-5-hydroxy-3,7- bis(methoxymethoxy)-2-(4- (methoxymethoxy)phenyl)-4H- chromen-4-one | 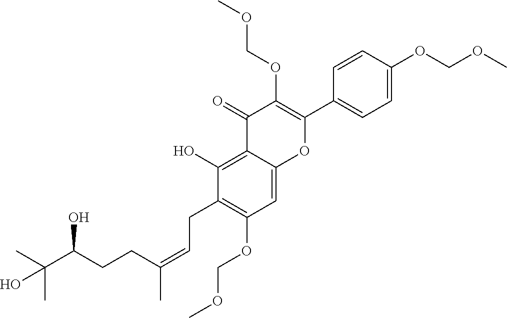 [SMILES: COCOc1ccc(-c2oc3cc(OCOC)c(C/C=C(/C)CC[C@H](O)C(C)(C)O)c(O)c3c(=O)c2OCOC)cc1] |
| VI-7 (R,E)-6-(6,7-dihydroxy-3,7- dimethyloct-2-en-1-yl)-5-hydroxy-3,7- bis(methoxymethoxy)-2-(4- (methoxymethoxy)phenyl)-4H- chromen-4-one | 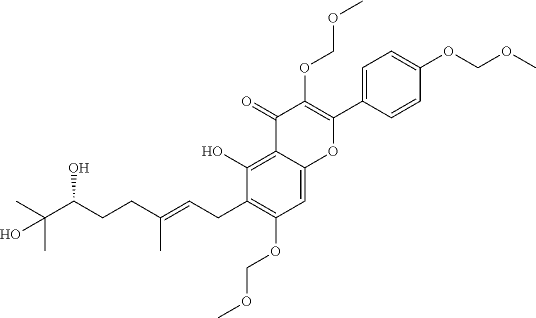 [SMILES: COCOc1ccc(-c2oc3cc(OCOC)c(C/C=C(\C)CC[C@@H](O)C(C)(C)O)c(O)c3c(=O)c2OCOC)cc1] |
| VI-8 stereoisomer (R,Z)-6-(6,7-dihydroxy-3,7- dimethyloct-2-en-1-yl)-5-hydroxy-3,7- bis(methoxymethoxy)-2-(4- (methoxymethoxy)phenyl)-4H- chromen-4-one | 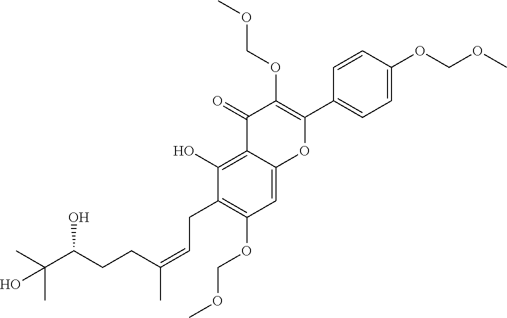 [SMILES: COCOc1ccc(-c2oc3cc(OCOC)c(C/C=C(/C)CC[C@@H](O)C(C)(C)O)c(O)c3c(=O)c2OCOC)cc1] |
| VI-9 (S,E)-6-(6,7-dihydroxy-3,7- dimethyloct-2-en-1-yl)-5-hydroxy-7- (methoxymethoxy)-2-(4- (methoxymethoxy)phenyl)-4H- chromen-4-one | 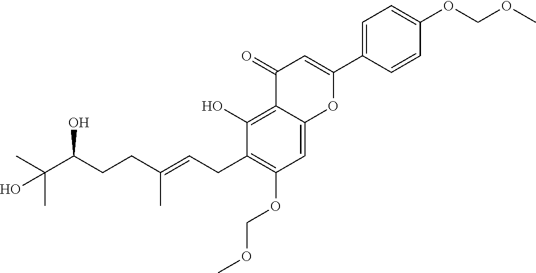 [SMILES: COCOc1ccc(-c2cc(=O)c3c(O)c(C/C=C(\C)CC[C@H](O)C(C)(C)O)c(OCOC)cc3o2)cc1] |
| VI-10 stereoisomer (S,Z)-6-(6,7-dihydroxy-3,7- dimethyloct-2-en-1-yl)-5-hydroxy-7- (methoxymethoxy)-2-(4- (methoxymethoxy)phenyl)-4H- chromen-4-one | 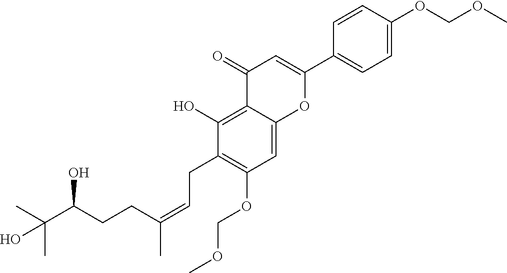 [SMILES: COCOc1ccc(-c2cc(=O)c3c(O)c(C/C=C(/C)CC[C@H](O)C(C)(C)O)c(OCOC)cc3o2)cc1] |

TABLE 8

| Number of the | |
|---|---|
| intermediate | Characterization (NMR and MS) |
| VI-1 | ¹H-NMR (500 MHz, CD₃CN): δ = 13.04 (s, 1 H), 8.00 (d, 2 H, J = |
| | 7.9 Hz), 7.60-7.56 (m, 3 H), 6.82 (s, 1 H), 6.76 (s, 1 H), 5.33 (s, 2 |
| | H), 5.26 (t, 1 H, J = 6.6 Hz), 3.47 (s, 3 H), 3.38 (d, 2 H, J = 6.8 Hz), |
| | 3.15 (dd, 1 H, J = 10.0, 5.0 Hz), 2.72 (d, 1 H, J = 4.7 Hz), 2.57 (s, 1 |
| | H), 2.23-1.94 (m, 4 H), 1.80 (s, 3 H), 1.05 (s, 3 H), 1.04 (s, 3 H) ppm |
| | ¹³C-NMR (125 MHz, CD₃CN): δ = 183.8 (1 C), 165.1 (1 C), |
| | 161.6 (1 C), 159.4 (1 C), 156.9 (1 C), 136.7 (1 C), 132.9 (1 C), |
| | 132.3 (1 C), 130.1 (2 C), 127.4 (2 C,), 122.8 (1 C), 114.3 (1 C), |
| | 106.8 (1 C), 106.5 (1 C), 95.2 (1 C), 93.7 (1 C), 78.6 (1 C), 73.1 |
| | (1 C), 56.9 (1 C), 37.6 (1 C), 30.7 (1 C), 26.1 (1 C), 24.6 (1 C), 22.2 |
| | (1 C), 16.4 (1 C) ppm. |
| | HRMS (ESI): m/z calculated for C₂₇H₃₃O₇⁺ [M + H]⁺: 469.2221; |
| | found: 469.2215 |
| VI-2 | ¹H-NMR (500 MHz, CD₃CN): δ = 13.04 (s, 1 H), 8.00 (d, 2 H, J = |
| stereoisomer | 7.9 Hz), 7.60-7.56 (m, 3 H), 6.82 (s, 1 H), 6.76 (s, 1 H), 5.33 (s, 2 |
| | H), 5.26 (t, 1 H, J = 6.6 Hz), 3.47 (s, 3 H), 3.38 (d, 2 H, J = 6.8 Hz), |
| | 3.15 (dd, 1 H, J = 10.0, 5.0 Hz), 2.72 (d, 1 H, J = 4.7 Hz), 2.57 (s, 1 |
| | H), 2.23-1.94 (m, 4 H), 1.67 (s, 3 H), 1.11 (s, 3 H), 1.09 (s, 3 H) ppm |
| | HRMS (ESI): m/z calculated for C₂₇H₃₃O₇⁺ [M + H]⁺: 469.2221; |
| | found: 469.2215 |
| VI-3 | ¹H-NMR (500 MHz, CD₃CN): δ = 13.04 (s, 1 H), 8.00 (d, 2 H, J = |
| | 7.9 Hz), 7.60-7.56 (m, 3 H), 6.82 (s, 1 H), 6.76 (s, 1 H), 5.33 (s, 2 |
| | H), 5.26 (t, 1 H, J = 6.6 Hz), 3.47 (s, 3 H), 3.38 (d, 2 H, J = 6.8 Hz), |
| | 3.15 (dd, 1 H, J = 10.0, 5.0 Hz), 2.72 (d, 1 H, J = 4.7 Hz), 2.57 (s, 1 |
| | H), 2.23-1.94 (m, 4 H), 1.80 (s, 3 H), 1.05 (s, 3 H), 1.04 (s, 3 H) ppm |
| | ¹³C-NMR (125 MHz, CD₃CN): δ = 183.8 (1 C), 165.1 (1 C), |
| | 161.6 (1 C), 159.4 (1 C), 156.9 (1 C), 136.7 (1 C), 132.9 (1 C), |
| | 132.3 (1 C), 130.1 (2 C), 127.4 (2 C,), 122.8 (1 C), 114.3 (1 C), |
| | 106.8 (1 C), 106.5 (1 C), 95.2 (1 C), 93.7 (1 C), 78.6 (1 C), 73.1 |
| | (1 C), 56.9 (1 C), 37.6 (1 C), 30.7 (1 C), 26.1 (1 C), 24.6 (1 C), 22.2 |
| | (1 C), 16.4 (1 C) ppm. |
| | HRMS (ESI): m/z calculated for C₂₇H₃₃O₇⁺ [M + H]⁺: 469.2221; |
| | found: 469.2215 |
| VI-4 | ¹H-NMR (500 MHz, CD₃CN): δ = 13.04 (s, 1 H), 8.00 (d, 2 H, J = |
| stereoisomer | 7.9 Hz), 7.60-7.56 (m, 3 H), 6.82 (s, 1 H), 6.76 (s, 1 H), 5.33 (s, 2 |
| | H), 5.26 (t, 1 H, J = 6.6 Hz), 3.47 (s, 3 H), 3.38 (d, 2 H, J = 6.8 Hz), |
| | 3.15 (dd, 1 H, J = 10.0, 5.0 Hz), 2.72 (d, 1 H, J = 4.7 Hz), 2.57 (s, 1 |
| | H), 2.23-1.94 (m, 4 H), 1.67 (s, 3 H), 1.11 (s, 3 H), 1.09 (s, 3 H) ppm |
| | HRMS (ESI): m/z calculated for C₂₇H₃₃O₇⁺ [M + H]⁺: 469.2221; |
| | found: 469.2215 |
| VI-5 | Not characterized |
| VI-6 stereoisomer | Not characterized |
| VI-7 | Not characterized |
| VI-8 stereoisomer | Not characterized |
| VI-9 | Not characterized |
| VI-10 stereoisomer | Not characterized |

According to a particular embodiment, in the compound of formula (VII), R₀ represents an oxygen atom; R₂ represents a linear or branched C₁-C₄ alkyl group; R₃ represents a linear or branched C₁-C₄ alkyl group; R₄ represents a linear or branched C₁-C₄ alkyl group; R₅ represents a protected hydroxyl group; R₆ represents a hydrogen atom or a protected hydroxyl group; R₇ represents a hydrogen atom or a protected hydroxyl group; R₅, R₉, R₁₀, and R₁₁ each represent a hydrogen atom; and R₁₂ represents a hydroxyl group or a protected hydroxyl group.

According to a particularly preferred embodiment, in the compound of formula (VI), R₀ represents an oxygen atom; R₂ represents a methyl group; R₃ represents a methyl group; R₄ represents a methyl group; R₅ represents a group —O—CH₂—O—CH₃; R₆ represents a hydrogen atom or a group —O—CH₂—O—CH₃; R₇ represents a hydrogen atom or a group —O—CH₂—O—CH₃; R₈, R₉, R₁₀, and R₁₁ each represent a hydrogen atom; and R₁₂ represents a hydroxyl group or a group —O—CH₂—O—CH₃.

According to another more preferred embodiment, the new compound of formula (VII) as defined in the invention is selected from the following compounds shown in Tables 9 and 10, characterized by NMR (nuclear magnetic resonance) and/or by mass spectrometry:

TABLE 9

| Number of the intermediate and | |
|---|---|
| chemical name | Structure of the compound |
| VII-1 (+/− racemic mixture) (E)-6-(5-(3,3-dimethyloxiran-2-yl)-3- methylpent-2-en-1-yl)-5,7- bis(methoxymethoxy)-2-phenyl-4H- chromen-4-one | 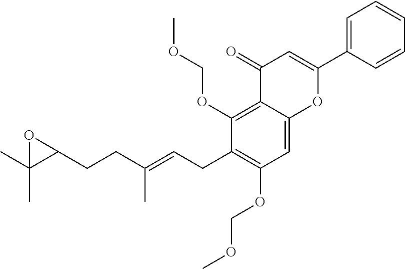 [SMILES: COCOc1cc2oc(-c3ccccc3)cc(=O)c2c(OCOC)c1C/C=C(\C)CCC1OC1(C)C] |
| VII-2 stereoisomer (+/− racemic mixture) (Z)-6-(5-(3,3-dimethyloxiran-2-yl)-3- methylpent-2-en-1-yl)-5,7- bis(methoxymethoxy)-2-phenyl-4H- chromen-4-one | 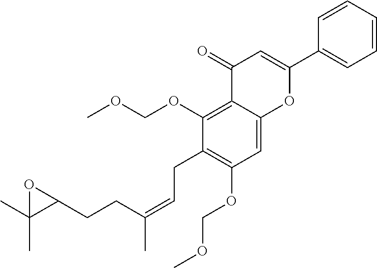 [SMILES: COCOc1cc2oc(-c3ccccc3)cc(=O)c2c(OCOC)c1C/C=C(/C)CCC1OC1(C)C] |
| VII-3 (R,E)-6-(5-(3,3-dimethyloxiran-2-yl)-3- methylpent-2-en-1-yl)-5-hydroxy-7- (methoxymethoxy)-2-phenyl-4H- chromen-4-one | 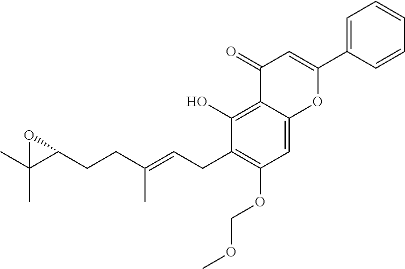 [SMILES: COCOc1cc2oc(-c3ccccc3)cc(=O)c2c(O)c1C/C=C(\C)CC[C@H]1OC1(C)C] |
| VII-4 stereoisomer (R,Z)-6-(5-(3,3-dimethyloxiran-2-yl)-3- methylpent-2-en-1-yl)-5-hydroxy-7- (methoxymethoxy)-2-phenyl-4H- chromen-4-one | 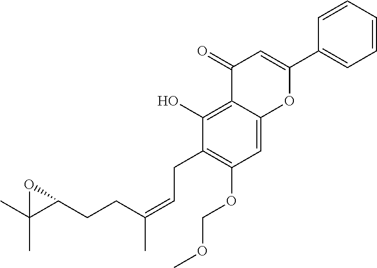 [SMILES: COCOc1cc2oc(-c3ccccc3)cc(=O)c2c(O)c1C/C=C(/C)CC[C@H]1OC1(C)C] |
| VII-5 (S,E)-6-(5-(3,3-dimethyloxiran-2-yl)-3- methylpent-2-en-1-yl)-5-hydroxy-7- (methoxymethoxy)-2-phenyl-4H- chromen-4-one | 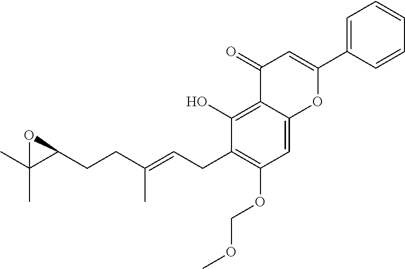 [SMILES: COCOc1cc2oc(-c3ccccc3)cc(=O)c2c(O)c1C/C=C(\C)CC[C@@H]1OC1(C)C] |
| VII-6 stereoisomer (S,Z)-6-(5-(3,3-dimethyloxiran-2-yl)-3- methylpent-2-en-1-yl)-5-hydroxy-7- (methoxymethoxy)-2-phenyl-4H- chromen-4-one | 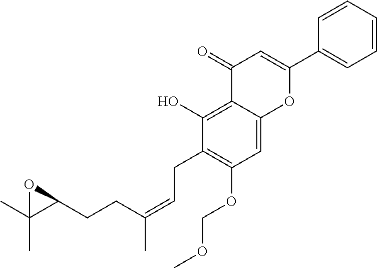 [SMILES: COCOc1cc2oc(-c3ccccc3)cc(=O)c2c(O)c1C/C=C(/C)CC[C@@H]1OC1(C)C] |
| VII-7 (R,E)-6-(5-(3,3-dimethyloxiran-2-yl)-3- methylpent-2-en-1-yl)-5-hydroxy-3,7- bis(methoxymethoxy)-2-(4- (methoxymethoxy)phenyl)-4H-chromen- 4-one | 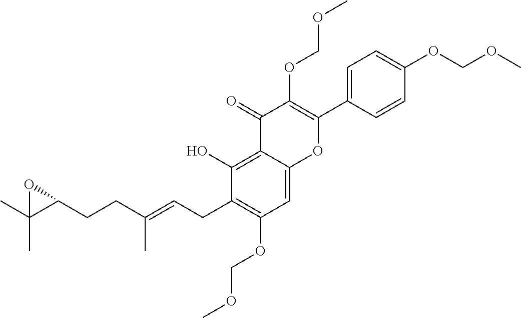 |
| VII-8 stereoisomer (R,Z)-6-(5-(3,3-dimethyloxiran-2-yl)-3- methylpent-2-en-1-yl)-5-hydroxy-3,7- bis(methoxymethoxy)-2-(4- (methoxymethoxy)phenyl)-4H-chromen- 4-one | 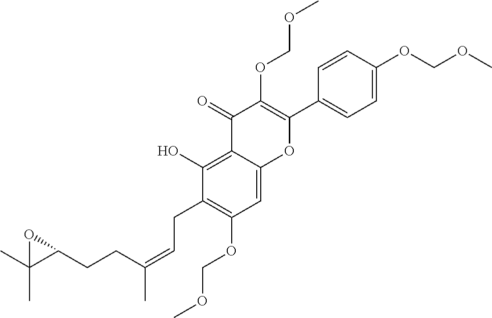 |
| VII-9 (S,E)-6-(5-(3,3-dimethyloxiran-2-yl)-3- methylpent-2-en-1-yl)-5-hydroxy-3,7- bis(methoxymethoxy)-2-(4- (methoxymethoxy)phenyl)-4H-chromen- 4-one | 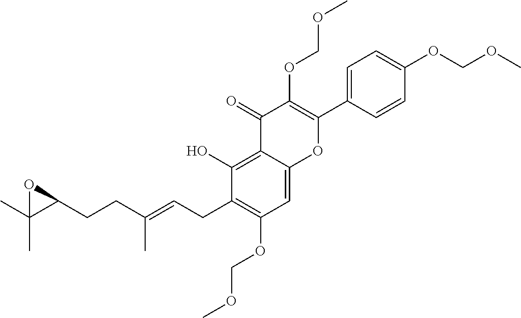 |
| VII-10 stereoisomer (S,Z)-6-(5-(3,3-dimethyloxiran-2-yl)-3- methylpent-2-en-1-yl)-5-hydroxy-3,7- bis(methoxymethoxy)-2-(4- (methoxymethoxy)phenyl)-4H-chromen- 4-one | 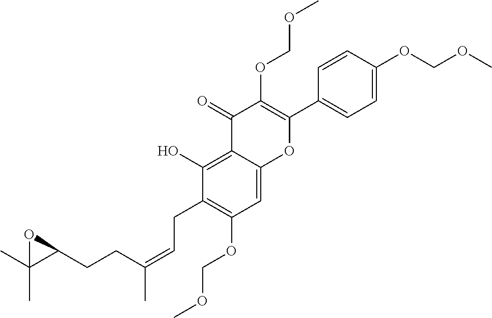 |
| VII-11 (R,E)-6-(5-(3,3-dimethyloxiran-2-yl)-3- methylpent-2-en-1-yl)-5-hydroxy-7- (methoxymethoxy)-2-(4- (methoxymethoxy)phenyl)-4H-chromen- 4-one | 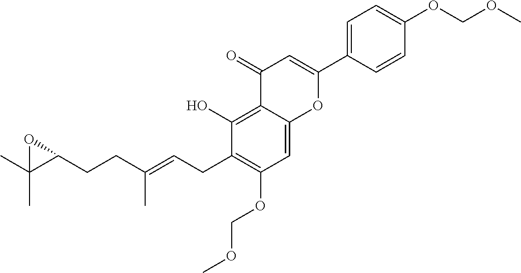 |
| VII-12 stereoisomer (R,Z)-6-(5-(3,3-dimethyloxiran-2-yl)-3- methylpent-2-en-1-yl)-5-hydroxy-7- (methoxymethoxy)-2-(4- (methoxymethoxy)phenyl)-4H-chromen- 4-one | 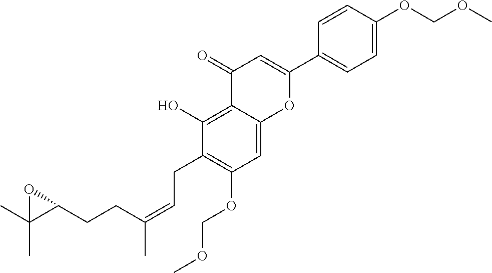 |

TABLE 10

| Number of the | |
|---|---|
| intermediate | Characterization (NMR and MS) |
| VII-1 | ¹H-NMR (500 MHz, CD₃CN): δ = 7.97 (dd, 2 H, J = 7.0, 2.3 Hz) |
| (+/− racemic | 7.56-7.52 (m, 3 H), 7.08 (s, 1 H), 6.63 (s, 1 H), 5.33 (s, 2 H), 5.26 (t, |
| mixture) | 1 H, J = 6.2 Hz), 5.10 (s, 2 H), 3.54 (s, 3 H), 3.51 (d, 2 H, J = 7.6 |
| | Hz), 3.46 (s, 3 H), 2.59 (t, 1H, J = 6.4 Hz), 2.13-2.06 (m, 2 H), 1.82 |
| | (s, 3 H), 1.62-1.52 (m, 2 H), 1.16 (s, 3 H), 1.15 (s, 3 H) ppm. |
| | ¹³C-NMR (125 MHz, CD₃CN): δ = 177.5 (1 C), 162.0 (1 C), |
| | 160.4 (1 C), 158.6 (1 C), 155.8 (1 C), 135.8 (1 C), 132.5 (2 C), |
| | 130.1 (2 C), 127.1 (2 C), 123.9 (1 C), 123.3 (1 C), 113.1 (1 C), |
| | 109.1 (1 C), 102.8 (1 C), 99.5 (1 C), 95.4 (1 C), 64.5 (1 C), 58.7 (1 |
| | C), 58.1 (1 C), 57.0 (1 C), 37.1 (1 C), 28.2 (1 C), 25.1 (1 C), 23.8 |
| | (1 C), 19.0 (1 C), 16.5 (1 C) ppm. |
| | HRMS (EST): m/z calculated for C₂₉H₃₅O₇⁺ [M + H]⁺: 495.2377; |
| | found: 495.2372 |
| VII-2 | ¹H-NMR (500 MHz, CD₃CN): δ = 7.97 (dd, 2 H′, J = 7.0, 2.3 Hz), |
| stereoisomer | 7.56-7.52 (m, 3 H), 7.08 (s, 1 H), 6.63 (s, 1 H), 5.33 (s, 2 H), 5.26 (t, |
| (+/− racemic | 1 H, J = 6.2 Hz), 5.10 (s, 2 H), 3.54 (s, 3 H), 3.51 (d, 2 H, J = 7.6 |
| mixture) | Hz), 3.46 (s, 3 H), 2.59 (t, 1 H, J = 6.4 Hz), 2.13-2.06 (m, 2 H), 1.68 |
| | (s, 3 H), 1.62-1.52 (m, 2 H), 1.27 (s, 3 H), 1.26 (s, 3 H) ppm. |
| | ¹³C-NMR (125 MHz, CD₃CN): δ = 177.5 (1 C), 162.0 (1 C), 160.4 |
| | (1 C), 158.6 (1 C), 155.8 (1 C), 135.8 (1 C), 132.5 (2 C), 130.1 (2 |
| | C), 127.1 (2 C), 123.9 (1 C), 123.3 (1 C), 113.1 (1 C), 109.1 (1 |
| | C), 102.8 (1 C), 99.5 (1 C), 95.4 (1 C), 64.7 (1 C), 58.8 (1 C), 58.1 |
| | (1 C), 57.0 (1 C), 37.6 (1 C), 29.5 (1 C), 25.2 (1 C), 23.6 (1 C), 22.0 |
| | (1 C), 19.0 (1 C) ppm. |
| | HRMS (ESI): m/z calculated for C₂₉H₃₅O₇⁺ [M + H]⁺: 495.2377; |
| | found: 495.2372 |
| VII-3 | ¹H-NMR (500 MHz, CD₃CN): δ = 13.00 (s, 1 H), 7.92 (d, 2 H, J = |
| | 7.7 Hz), 7.56-7.49 (m, 3 H), 6.73 (s, 1 H), 6.67 (s, 1 H), 5.28 (s, 2 H), |
| | 5.25 (t, 1 H, J = 7.2 Hz), 3.45 (s, 3 H), 3.31 (d, 2 H, J = 7.6 Hz), 2.58 |
| | (t, 1 H, J = 6.4 Hz), 2.10-2.05 (m, 2 H), 1.79 (s, 3 H), 1.59-1.51 (m, |
| | 2 H), 1.15 (s, 3 H), 1.13 (s, 3 H) ppm. |
| | ¹³C-NMR (125 MHz, CD₃CN): δ = 183.6 (1 C), 164.9 (1 C), |
| | 161.6 (1 C), 159.3 (1 C), 156.8 (1 C), 135.6 (1 C), 132.8 (1 C), |
| | 132.2 (1 C), 130.0 (2 C), 127.3 (2 C), 123.3 (1 C), 114.0 (1 C), |
| | 106.6 (1 C), 106.4 (1 C), 95.2 (1 C), 93.5 (1 C), 64.5 (1 C), 58.7 |
| | (1 C), 56.9 (1 C), 37.0 (1 C), 28.1 (1 C), 25.0 (1 C), 22.2 (1 C), 18.9 |
| | (1 C), 16.3 (1 C) ppm. |
| | HRMS (ESI): m/z calculated for C₂₇H₃₁O₆⁺ [M + H]⁺: 451.2115; |
| | found: 451.2119 |
| VII-4 | ¹H-NMR (500 MHz, CD₃CN): δ = 13.00 (s, 1 H), 7.92 (d, 2 H, J = |
| stereoisomer | 7.7 Hz), 7.56-7.49 (m, 3 H), 6.73 (s, 1 H), 6.67 (s, 1 H), 5.28 (s, 2 H), |
| | 5.25 (t, 1 H, J = 7.2 Hz), 3.45 (s, 3 H), 3.31 (d, 2 H, J = 7.6 Hz), 2.58 |
| | (t, 1 H, J = 6.4 Hz), 2.10-2.05 (m, 2 H), 1.68 (s, 3 H), 1.59-0.51 (m, |
| | 2 H), 1.27 (s, 3 H), 1.26 (s, 3 H) ppm. |
| | ¹³C-NMR (125 MHz, CD₃CN): δ = 183.6 (1 C), 164.9 (1 C), 161.6 |
| | (1 C), 159.3 (1 C), 156.8 (1 C), 136.0 (1 C), 132.8 (1 C), 132.2 (1 |
| | C), 130.0 (2 C), 127.3 (2 C), 123.9 (1 C), 114.0 (1 C), 106.6 (1 |
| | C), 106.4 (1 C), 95.2 (1 C), 93.5 (1 C), 64.6 (1 C), 58.8 (1 C), 56.9 |
| | (1 C), 37.6 (1 C), 29.3 (1 C), 25.2 (1 C), 23.6 (1 C), 22.0 (1 C), 19.0 |
| | (1 C) ppm. |
| | HRMS (ESI): m/z calculated for C₂₇H₃₁O₆⁺ [M + H]⁺: 451.2115; |
| | found: 451.2119 |
| VII-5 | ¹H-NMR (500 MHz, CD₃CN): δ = 13.00 (s, 1 H), 7.92 (d, 2 H, J = |
| | 7.7 Hz), 7.56-7.49 (m, 3 H), 6.73 (s, 1 H), 6.67 (s, 1 H), 5.28 (s, 2 H), |
| | 5.25 (t, 1 H, J = 7.2 Hz), 3.45 (s, 3 H), 3.31 (d, 2 H, J = 7.6 Hz), 2.58 |
| | (t, 1 H, J = 6.4 Hz), 2.10-2.05 (m, 2 H), 1.79 (s, 3 H), 1.59-1.51 (m, |
| | 2 H), 1.15 (s, 3 H), 1.13 (s, 3 H) ppm. |
| | ¹³C-NMR (125 MHz, CD₃CN): δ = 183.6 (1 C), 164.9 (1 C), |
| | 161.6 (1 C), 159.3 (1 C), 156.8 (1 C), 135.6 (1 C), 132.8 (1 C), |
| | 132.2 (1 C), 130.0 (2 C), 127.3 (2 C), 123.3 (1 C), 114.0 (1 C), |
| | 106.6 (1 C), 106.4 (1 C), 95.2 (1 C), 93.5 (1 C), 64.5 (1 C), 58.7 |
| | (1 C), 56.9 (1 C), 37.0 (1 C), 28.1 (1 C), 25.0 (1 C), 22.2 (1 C), 18.9 |
| | (1 C), 16.3 (1 C) ppm. |
| | HRMS (ESI): m/z calculated for C₂₇H₃₁O₆⁺ [M + H]⁺: 451.2115; |
| | found: 451.2119. |
| VII-6 | ¹H-NMR (500 MHz, CD₃CN): δ = 13.00 (s, 1 H), 7.92 (d, 2 H, J = |
| stereoisomer | 7.7 Hz), 7.56-7.49 (m, 3 H), 6.73 (s, 1 H), 6.67 (s, 1 H), 5.28 (s, 2 H), |
| | 5.25 (t, 1 H, J = 7.2 Hz), 3.45 (s, 3 H), 3.31 (d, 2 H, J = 7.6 Hz), 2.58 |
| | (t, 1 H, J = 6.4 Hz), 2.10-2.05 (m, 2 H), 1.68 (s, 3 H), 1.59-0.51 (m, |
| | 2 H), 1.27 (s, 3 H), 1.26 (s, 3 H) ppm. |
| | ¹³C-NMR (125 MHz, CD₃CN): δ = 183.6 (1 C), 164.9 (1 C), 161.6 |
| | (1 C), 159.3 (1 C), 156.8 (1 C), 136.0 (1 C), 132.8 (1 C), 132.2 (1 |
| | C), 130.0 (2 C), 127.3 (2 C), 123.9 (1 C), 114.0 (1 C), 106.6 (1 |
| | C), 106.4 (1 C), 95.2 (1 C), 93.5 (1 C), 64.6 (1 C), 58.8 (1 C), 56.9 |
| | (1 C), 37.6 (1 C), 29.3 (1 C), 25.2 (1 C), 23.6 (1 C), 22.0 (1 C), 19.0 |
| | (1 C) ppm. |
| | HRMS (ESI): m/z calculated for C₂₇H₃₁O₆⁺ [M + H]⁺: 451.2115; |
| | found: 451.2119 |
| VII-7 | ¹H-NMR (500 MHz, CD₃CN): δ = 12.78 (s, 1 H), 8.02 (d, 2 H, J = |
| | 8.3 Hz), 7.14 (dd, 2 H, J = 8.3 Hz), 6.69 (s, 1 H), 5.28 (s, 2 H), 5.25 |
| | (s, 3 H), 5.11 (s, 2 H), 3.44 (s, 6 H), 3.34 (d, 2 H, J = 6.9 Hz), 3.19 |
| | (s, 3 H), 2.58 (t, 1 H, J = 5.5 Hz), 2.13-2.02 (m, 2 H), 1.80 (s, 3 H), |
| | 1.66-1.45 (m, 2 H), 1.15 (s, 3 H), 1.14 (s, 3 H) ppm. |
| | ¹³C-NMR (125 MHz, CD₃CN): δ = 179.6 (1 C), 161.5 (1 C), |
| | 160.2 (1 C), 159.0 (1 C), 157.5 (1 C), 155.9 (1 C), 136.6 (1 C), |
| | 135.7 (1 C), 131.5 (2 C), 124.8 (1 C), 123.3 (1 C), 116.9 (2 C), |
| | 113.5 (1 C), 106.9 (1 C), 98.8 (1 C), 95.2 (1 C), 95.1 (1 C), 93.3 (1 |
| | C), 64.5 (1 C), 58.6 (1 C), 58.0 (1 C), 56.9 (1 C), 56.6 (1 C), 37.0 |
| | (1 C), 28.1 (1 C), 24.9 (1 C), 22.2 (1 C), 18.9 (1 C), 16.3 (1 C) ppm. |
| | HRMS (EST): m/z calculated for C₃₁H₃₉O₁₀⁺ [M + H]⁺: 571.2538; |
| | found: 571.2541. |
| VII-8 | ¹H-NMR (500 MHz, CD₃CN): δ = 12.80 (s, 1 H), 8.02 (d, 2 H, J = ) |
| stereoisomer | 8.3 Hz), 7.14 (d, 2 H, J = 8.3 Hz), 6.69 (s, 1 H), 5.28 (s, 2 H), 5.25 (s, |
| | 3 H), 5.11 (s, 2 H), 3.44 (s, 6 H), 3.34 (d, 2 H, J = 6.9 Hz), 3.19 (s, 3 |
| | H), 2.58 (t, 1 H, J = 5.5 Hz), 2.13-2.02 (m, 2 H), 1.67 (s, 3 H), 1.66- |
| | 1.45 (m, 2 H), 1.27 (s, 3 H), 1.26 (s, 3 H) ppm. |
| | ¹³C-NMR (125 MHz, CD₃CN): δ = 179.6 (1 C), 161.5 (1 C), |
| | 160.2 (1 C), 159.0 (1 C), 157.5 (1 C), 155.9 (1 C), 136.6 (1 C), |
| | 136.0 (1 C), 131.5 (2 C), 124.8 (1 C), 123.9 (1 C), 116.9 (2 C), |
| | 113.5 (1 C), 106.9 (1 C), 98.8 (1 C), 95.2 (1 C), 95.1 (1 C), 93.3 (1 |
| | C), 64.6 (1 C), 58.6 (1 C), 58.0 (1 C), 56.9 (1 C), 56.6 (1 C), 36.9 |
| | (1 C), 28.3 (1 C), 25.1 (1 C), 22.0 (1 C), 19.0 (1 C), 16.3 (1 C) ppm. |
| | HRMS (ESI): m/z calculated for C₃₁H₃₉O₁₀⁺ [M + H]⁺: 571.2538; |
| | found: 571.2541. |
| VII-9 | ¹H-NMR (500 MHz, CD₃CN): δ = 12.78 (s, 1 H), 8.02 (d, 2 H, J = |
| | 8.3 Hz), 7.14 (dd, 2 H, J = 8.3 Hz), 6.69 (s, 1 H), 5.28 (s, 2 H), 5.25 |
| | (s, 3 H), 5.11 (s, 2 H), 3.44 (s, 6 H), 3.34 (d, 2 H, J = 6.9 Hz), 3.19 |
| | (s, 3 H), 2.58 (t, 1 H, J = 5.5 Hz), 2.13-2.02 (m, 2 H), 1.80 (s, 3 H), |
| | 1.66-1.45 (m, 2 H), 1.15 (s, 3 H), 1.14 (s, 3 H) ppm. |
| | ¹³C-NMR (125 MHz, CD₃CN): δ = 179.6 (1 C), 161.5 (1 C), |
| | 160.2 (1 C), 159.0 (1 C), 157.5 (1 C), 155.9 (1 C), 136.6 (1 C), |
| | 135.7 (1 C), 131.5 (2 C), 124.8 (1 C), 123.3 (1 C), 116.9 (2 C), |
| | 113.5 (1 C), 106.9 (1 C), 98.8 (1 C), 95.2 (1 C), 95.1 (1 C), 93.3 (1 |
| | C), 64.5 (1 C), 58.6 (1 C), 58.0 (1 C), 56.9 (1 C), 56.6 (1 C), 37.0 |
| | (1 C), 28.1 (1 C), 24.9 (1 C), 22.2 (1 C), 18.9 (1 C), 16.3 (1 C) ppm. |
| | HRMS (ESI): m/z calculated for C₃₁H₃₉O₁₀⁺ [M + H]⁺: 571.2538; |
| | found: 571.2541. |
| VII-10 | ¹H-NMR (500 MHz, CD₃CN): δ = 12.80 (s, 1 H), 8.02 (d, 2 H, J = |
| stereoisomer | 8.3 Hz), 7.14 (d, 2 H, J = 8.3 Hz), 6.69 (s, 1 H), 5.28 (s, 2 H), 5.25 (s, |
| | 3 H), 5.11 (s, 2 H), 3.44 (s, 6 H), 3.34 (d, 2 H, J = 6.9 Hz), 3.19 (s, 3 |
| | H), 2.58 (t, 1 H, J = 5.5 Hz), 2.13-2.02 (m, 2 H), 1.67 (s, 3 H), 1.66- |
| | 1.45 (m, 2 H), 1.27 (s, 3 H), 1.26 (s, 3 H) ppm. |
| | ¹³C-NMR (125 MHz, CD₃CN): δ = 179.6 (1 C), 161.5 (1 C), |
| | 160.2 (1 C), 159.0 (1 C), 157.5 (1 C), 155.9 (1 C), 136.6 (1 C), |
| | 136.0 (1 C), 131.5 (2 C), 124.8 (1 C), 123.9 (1 C), 116.9 (2 C), |
| | 113.5 (1 C), 106.9 (1 C), 98.8 (1 C), 95.2 (1 C), 95.1 (1 C), 93.3 (1 |
| | C), 64.6 (1 C), 58.6 (1 C), 58.0 (1 C), 56.9 (1 C), 56.6 (1 C), 36.9 |
| | (1 C), 28.3 (1 C), 25.1 (1 C), 22.0 (1 C), 19.0 (1 C), 16.3 (1 C) ppm. |
| | HRMS (EST): m/z calculated for C₃₁H₃₉O₁₀⁺ [M + H]⁺: 571.2538; |
| | found: 571.2541. |
| VII-11 | ¹H-NMR (500 MHz, CD₃CN): δ = 13.04 (s, 1H), 7.85 (d, 2 H, J = |
| | 8.3 Hz), 7.11 (d, 2 H, J = 8.3 Hz), 6.70 (s, 1 H), 6.56 (s, 1 H), 5.27 (s, |
| | 2 H), 5.25 (m, 1 H), 5.24 (s, 2 H), 3.45 (s, 3 H), 3.44 (s, 3 H), 3.30 |
| | (d, 2 H, J = 6.6 Hz), 2.58 (t, 1 H, J = 5.8 Hz), 2.13-2.02 (m, 2 H), |
| | 1.79 (s, 3 H), 1.64-1.50 (m, 2 H), 1.15 (s, 3 H), 1.13 (s, 3 H) ppm. |
| | ¹³C-NMR (125 MHz, CD₃CN): δ = 183.5 (1 C), 164.7 (1 C), |
| | 161.4 (1 C), 161.1 (1 C), 159.3 (1 C), 156.7 (1 C), 135.6 (1 C), |
| | 129.0 (2 C), 125.3 (1 C), 123.3 (1 C), 117.4 (2 C), 113.9 (1 C), |
| | 106.4 (1 C), 105.0 (1 C), 95.3 (1 C), 95.2 (1 C), 93.4 (1 C), 64.5 |
| | (1 C), 58.7 (1 C), 56.9 (1 C), 56.6 (1 C), 37.0 (1 C), 28.1 (1 C), 24.9 |
| | (1 C), 23.5 (1 C), 18.9 (1 C), 16.3 (1 C) ppm. |
| | HRMS (ESI): m/z calculated for C₂₉H₃₅O₈⁺ [M + H]⁺: 511.2326; |
| | found: 511.2326. |
| VII-12 | ¹H-NMR (500 MHz, CD₃CN): δ = 13.05 (s, 1H), 7.85 (d, 2 H, J = |
| stereoisomer | 8.3 Hz), 7.11 (d, 2 H, J = 8.3 Hz), 6.54 (s, 1 H), 6.43 (s, 1 H), 5.27 (s, |
| | 2 H), 5.25 (m, 1 H), 5.24 (s, 2 H), 3.45 (s, 3 H), 3.44 (s, 3 H), 3.30 |
| | (d, 2 H, J = 6.6 Hz), 2.73 (t, 1 H, J = 6.2 Hz), 2.13-2.02 (m, 2 H), |
| | 1.67 (s, 3 H), 1.64-1.50 (m, 2 H), 1.27 (s, 3 H), 1.26 (s, 3 H) ppm. |
| | ¹³C-NMR (125 MHz, CD₃CN): δ = 183.5 (1 C), 164.7 (1 C), 161.4 |
| | (1 C), 161.1 (1 C), 159.2 (1 C), 156.7 (1 C), 135.9 (1 C), 129.0 (2 |
| | C), 125.3 (1 C), 123.9 (1 C), 117.4 (2 C), 113.8 (1 C), 106.4 (1 |
| | C), 105.0 (1 C), 95.3 (1 C), 95.2 (1 C), 93.4 (1 C), 64.6 (1 C), 58.8 |
| | (1 C), 56.9 (1 C), 56.6 (1 C), 36.9 (1 C), 28.3 (1 C), 25.1 (1 C), 22.2 |
| | (1 C), 19.0 (1 C), 16.5 (1 C) ppm. |
| | HRMS (ESI): m/z calculated for C₂₉H₃₅O₈⁺ [M + H]⁺: 511.2326; |
| | found: 511.2326. |

In the context of the present invention, like the compound of formula (I) according to the invention, the pharmaceutically acceptable salts, they hydrates, the solvates, as well as the tautomeric forms, stereoisomers, mixtures of stereoisomers, pure enantiomers and mixtures whether racemic or not are also included in the definition of intermediates of formula (IV), (V), (VI) or (VII) according to the invention.

### Pharmaceutical Compositions According to the Invention

The present invention also relates to a pharmaceutical composition comprising at least one compound of formula (I) as defined according to the invention and at least one pharmaceutically acceptable excipient.

As an example, it may comprise an amount of compounds of formula (I) as defined in the invention from 0.05 to 10 wt %, in particular from 0.1 to 5 wt %, relative to the total weight of the composition.

The composition according to the invention further comprises at least one pharmaceutically acceptable excipient.

This excipient may be solid or liquid. It may be selected, for example, from purified water, ethyl alcohol, propylene glycol, glycerin, vegetable oils, animal oils, hydrocarbons, silicones, sugars such as glucose, levulose, wheat starch, maize starch, potato starch, cyclodextrins, xanthan gum, pectins, alginates, magnesium stearate, gelatin, cellulose and derivatives thereof.

The composition of the invention may be administered by any suitable route, for example by the oral, rectal, local (topical, for example), intraperitoneal, systemic, intravenous, intramuscular, subcutaneous or mucosal, in particular sublingual, route or else using a patch, or else in encapsulated form in, or immobilized on, liposomes, microparticles, microcapsules, combined with nanoparticles and the like.

The excipients suitable for the present invention may be any excipient used conventionally in the field of therapy.

We may mention in particular, as nonlimiting examples of suitable excipients for administration by the oral route, talc, lactose, starch and derivatives thereof, cellulose and derivatives thereof, polyethylene glycols, polymers of acrylic acid, gelatin, magnesium stearate, animal, vegetable or synthetic fats, paraffin derivatives, glycols, stabilizers, preservatives, antioxidants, wetting agents, anti-agglomerants, dispersants, emulsifiers, taste modifying agents, penetrating agents and solubilizers. For example, to prepare an aqueous solution that is injectable by the intravenous route, a cosolvent may be used, for example such as an alcohol such as ethanol or a glycol such as polyethylene glycol or propylene glycol. According to another example, to prepare an oily solution that is injectable by the intramuscular route, at least one compound of formula (I) may be solubilized using a triglyceride or a glycerol ester.

The pharmaceutical composition may thus comprise one or more pharmaceutically acceptable diluents, carriers, excipients, fillers, binders, lubricants, glidants, disintegrants, absorbents and/or preservatives.

The techniques for formulation and administration of drugs and pharmaceutical compositions are well known by a person skilled in the art considered here.

According to the invention, the composition may advantageously be administered by the oral route or by intravenous injection.

A therapeutically effective dose for the pharmaceutical compositions according to the invention is determined by standard clinical techniques as assessed by the doctor.

Advantageously, the composition according to the invention is suitable for administration by the oral or intravenous route at a dose greater than or equal to 1 mg/kg/24 h and less than or equal to 200 mg/kg/24 h in one or more doses to a mammal needing same.

Thus, the compounds of formula (I) of the invention may be used at doses between 0.01 mg and 5000 mg per day, given in a single dose once daily or administered in several doses throughout the day, for example twice daily in equal doses. The dose administered per day is advantageously between 5 mg and 2500 mg, even more advantageously between 10 mg and 1000 mg.

The suitable dosage forms for administration by the oral route comprise tablets, capsules, powders, granules, syrups and oral solutions or suspensions.

For parenteral administration, it is possible to use aqueous suspensions, isotonic saline solutions or sterile injectable solutions that contain pharmaceutically acceptable and compatible dispersants and/or wetting agents.

According to a particular embodiment, the pharmaceutical composition as defined in the present invention may further comprise another active ingredient, useful in particular in the treatment and/or prevention of neurodegenerative diseases, viral diseases, dyslipidemia, hypercholesterolemia and/or cancers.

Thus, the present invention also relates to a pharmaceutical preparation that comprises a pharmaceutical composition according to the invention, and in addition, in a mixture or packaged separately, at least one antiviral agent and/or anticancer agent and/or agent effective against neurodegenerative diseases, and/or an agent effective against dyslipidemia, and/or an agent effective against hypercholesterolemia for use thereof for treating and/or preventing and/or inhibiting infections caused by pathogens such as viruses, cancers, neurodegenerative diseases, hypercholesterolemia and dyslipidemia, administered simultaneously, sequentially, or at intervals.

### Therapeutic Applications

As mentioned above, the present invention relates to a compound of formula (I) according to the invention or a pharmaceutical composition according to the invention for use as a medicinal product, and in particular for use for preventing and/or treating and/or inhibiting cancers, neurodegenerative diseases, dyslipidemia, hypercholesterolemia and/or viral diseases.

The compound of formula (I) as defined in the present invention makes it possible to inhibit OSBP (oxysterol-binding protein), a protein responsible for intracellular transfer of cholesterol.

According to a particular embodiment, the cancers are selected from breast cancer, including triple-negative breast cancer, kidney cancer, head and neck cancer, prostate cancer, colorectal cancer, colon cancer, gallbladder cancer, biliary tract cancer (cholangiocarcinoma), gastrointestinal cancer, gastric cancer, hepatocellular carcinoma, lymphoma, lung cancer, small-cell lung cancer, nonsmall cell lung cancer, pancreatic cancer, pancreatic carcinoma, stomach cancer, brain cancer, metastases, leukemia, T-cell acute lymphoblastic leukemia, chronic myeloid leukemia, melanoma, and glioblastoma, in particular kidney cancer, triple-negative breast cancer, melanoma, leukemia and glioblastoma.

Glioblastoma or glioblastoma multiforme (GBM), also known as grade 4 astrocytoma, is the commonest and most aggressive brain tumor.

According to another particular embodiment, the neurodegenerative diseases are selected from amyotrophic lateral sclerosis (ALS or Charcot disease), Alzheimer's disease, Parkinson's disease, multiple sclerosis, Huntington's disease (Huntington's chorea), and Niemann-Pick disease type C, in particular Alzheimer's disease, Parkinson's disease, Huntington's disease (Huntington's chorea), and Niemann-Pick disease type C.

In the present invention, “viral diseases” means any benign or serious disease caused by a virus.

According to one embodiment of the invention, the viral diseases targeted by the compounds of the invention are caused by RNA viruses.

According to an even more particular embodiment, a viral infection with RNA viruses means more particularly, in the context of the present invention, a viral infection caused by a virus belonging to group III (Viruses with double-stranded RNA (dsRNA)), group IV (Viruses with single-stranded RNA with positive polarity: RNA with polarity (+)), group V (Viruses with single-stranded RNA with negative polarity: RNA with polarity (−)) or group VI (Retroviruses with single-stranded RNA: RNA with polarity (+) with intermediate DNA in the life cycle).

According to this classification, the viruses belonging to group VI are not strictly speaking RNA viruses. A well-studied example of a family of viruses belonging to group VI is the family Retroviridae (retroviruses), which includes HIV.

As representatives of viruses belonging to group III, we may mention the Reoviridae and the Birnaviridae.

As representatives of viruses belonging to group IV, we may mention the Picornaviridae (which is a family of viruses that includes well-known viruses such as the hepatitis A virus, enteroviruses, rhinoviruses, poliovirus and foot-and-mouth disease virus), SARS virus, hepatitis C virus, yellow fever virus and rubella virus. The family Togaviridae also belongs to group IV and a known genus of the latter is alphavirus, which includes the Chikungunya virus. The Flaviridae are also a family belonging to group IV, including a famous virus transmitted by mosquitoes, namely Dengue virus or Zika virus. An acute respiratory disease has recently been caused by a new coronavirus (SARS-CoV-2, known previously by the name 2019-nCoV), also called coronavirus disease 2019 here (COVID-19), which belongs to the Coronaviridae and forms part of group IV of the Baltimore classification.

As representatives of viruses belonging to group V, we may mention the Filoviridae family of viruses which includes the Ebola virus, the family Paramyxoviridae which includes respiratory syncytial virus (RSV), the family Rhabdoviridae, the family Orthomyxoviridae comprising influenza virus A, influenza virus B and influenza C. Measles is also caused by a virus in group V, of the family of the paramwyxoviruses.

According to another embodiment of the invention, the viral diseases targeted by the compounds of the invention are infections caused by the RNA viruses of classes IV and V of the Baltimore classification. Viral pharyngitis may be caused by various viruses such as rhinoviruses, coronaviruses, respiratory syncytial virus (RSV), influenza virus and para-influenza virus.

According to yet another particular embodiment, the viral diseases targeted by the compounds of the invention are selected from dengue, Zika virus infection, influenza, viral pharyngitis, measles, AIDS, Chikungunya, yellow fever, poliomyelitis, hepatitis A, hepatitis C, hepatitis E, infection with SARS-CoV virus, infection with SARS-CoV-2 virus, and rubella, in particular dengue, Zika virus infection, chikungunya, yellow fever, poliomyelitis, hepatitis A, hepatitis C, hepatitis E, infection with SARS-CoV virus, infection with SARS-CoV-2 virus and rubella.

Thus, according to a particularly preferred embodiment, the viral diseases are in particular those caused by a virus with (+) strand RNA—Baltimore Class IV such as dengue, Zika virus infection, chikungunya, yellow fever, poliomyelitis, hepatitis A, hepatitis C, hepatitis E, infection with SARS-CoV virus, infection with SARS-CoV-2 virus, and rubella.

Other viral infections, not caused by RNA viruses, may also be targeted in the context of the present invention, such as chickenpox.

As already mentioned above, a compound of formula (I) as defined in the invention may be administered alone or in combination with other active ingredients that are able to act synergistically with the compound of formula (I).

Moreover, other treatment techniques may be combined with the treatment carried out by administering a pharmaceutical composition according to the invention or a medicinal product comprising at least one compound of formula (I).

For example, when the pathology in question is a cancer, it is possible to combine radiotherapy, optionally coupled with chemotherapy, with other active ingredients firstly, and then continue the treatment as monotherapy by administering a pharmaceutical composition according to the invention or a medicinal product comprising at least one compound of formula (I).

The present invention also relates to a method for administering at least one compound of formula (I) as defined in the invention to a subject requiring it, comprising:
Supplying a pharmaceutical composition comprising at least one compound of formula (I) as defined in the invention, and at least one pharmaceutically acceptable excipient; and
Administering the pharmaceutical composition in an effective amount to a subject requiring it.

According to other embodiments, the present invention relates to methods of administration, in particular by the oral or intravenous route, of a pharmaceutical composition according to the invention comprising at least one compound of formula (I) as defined in the invention and an additional active ingredient.

A “subject” (including a patient) includes mammals (for example humans, pets, farm animals or laboratory animals).

According to another aspect, the present invention relates to the use of at least one compound of formula (I) as defined in the invention or of a pharmaceutical composition according to the invention for preparing a medicinal product.

According to another aspect, the present invention relates to the use of at least one compound of formula (I) as defined in the invention or of a pharmaceutical composition according to the invention for preparing a medicinal product for treating and/or preventing and/or inhibiting cancers, neurodegenerative diseases, dyslipidemia, hypercholesterolemia, and/or viral diseases.

According to yet another aspect, the present invention relates to a method of treatment and/or prevention and/or inhibition of cancers, neurodegenerative diseases, dyslipidemia, hypercholesterolemia, and/or viral diseases, comprising the administration, to a subject requiring it, of a pharmaceutical composition according to the invention comprising at least one compound of formula (I) as defined in the present invention.

According to yet another aspect, the present invention relates to a method of treatment and/or prevention and/or inhibition of cancers, neurodegenerative diseases, dyslipidemia, hypercholesterolemia, and/or viral diseases, comprising administration, to a subject requiring it, of a therapeutically effective amount of at least one compound of formula (I) as defined in the present invention.

The expressions “between . . . and . . . ” and “from . . . to . . . ” are to be understood as inclusive, unless stated otherwise.

In the description and the examples, temperature is expressed in degrees Celsius unless stated otherwise, and pressure is atmospheric pressure, unless stated otherwise.

The invention will now be described by means of the following examples, given of course for purposes of illustration, and not limiting the invention.

### EXAMPLES

### Materials and Methods

The proton nuclear resonance spectra (¹H NMR) are recorded in CD₃OD, CD₃CN, CDCl₃ and deuterated DMSO on a Bruker Avance instrument (300 MHz or 500 MHz). The chemical shifts (δ) are expressed in parts per million (ppm) relative to the residue of undeuterated solvent. The coupling constants (J) are in hertz (Hz). The following abbreviations are used for the multiplicity of the signals: s (singlet), d (doublet), t (triplet), dd (doublet of doublet), dt (doublet of triplet), td (triplet of doublet), q (quadruplet), m (multiplet).

The carbon magnetic resonance spectra (¹³C NMR) are recorded on a Bruker Avance instrument (75 or 125 MHz); the chemical shifts are expressed in ppm relative to the solvents used.

The high-resolution mass spectra (HRMS) are obtained on a Waters LCT Premier XE spectrometer in ESI mode (electrospray ionization). Purifications by flash chromatography are carried out on a Teledyne Isco Combiflash Rf 200i with GraceResolv prepackaged silica cartridges.

All the reagents are commercial, unless otherwise, when they are stated specifically. When necessary, the organic solvents are dried or distilled before use and stored on a molecular sieve under argon.

### Example 1: Preparation of the Compounds of Formula (I) Designated 1, 2, 3 and 12 According to the Invention

### Protection of chrysin II-1 (step (B))

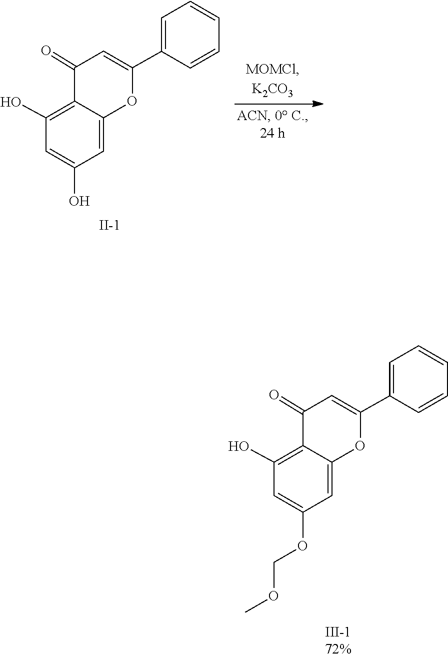

A mixture of chrysin II-1 (5.0 g, 19.7 mmol, 1.0 eq) and potassium carbonate (K₂CO₃: 13.6 g, 98.3 mmol, 5.0 eq) dissolved in 197 ml of acetonitrile is placed under an argon atmosphere and cooled to 0° C. for 30 min. Methyl chloromethyl ether (MOMCl: 2.6 ml, 34.8 mmol, 1.8 eq) is added dropwise, at 0° C., over a period of 1 hour. The mixture is stirred at the same temperature for 2 hours and then 197 ml of water is added. After decanting, the aqueous phase is extracted with ethyl acetate three times. The collected organic phases are washed with saturated aqueous solution of sodium chloride, and then dried over magnesium sulfate, filtered and concentrated at reduced pressure. The crude product is purified by silica column chromatography using a heptane/EtOAc gradient (95:5 to 0:100) to give the pure compound III-1 (4.2 g, 14.1 mmol, 72%) in the form of a yellow solid.

### Iodation of compound III-1 (step (C)) and formation of compound IV-4 (step of protection of a hydroxyl group)

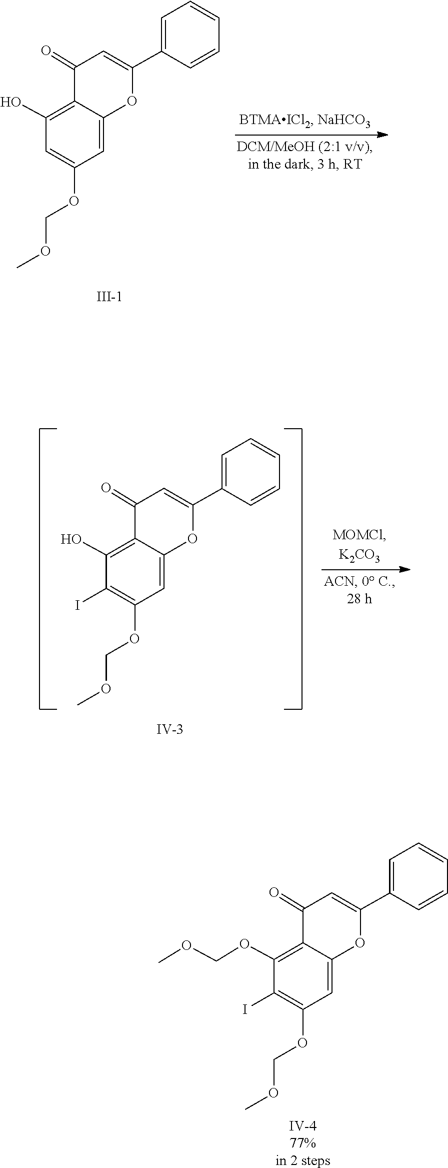

A mixture of compound III-1 (2.7 g, 9.0 mmol, 1.0 eq), sodium hydrogen carbonate (NaHCO₃: 7.5 g, 89.7 mmol, 10.0 eq) and benzyltrimethylammonium dichloroiodate (BTMA-ICl2:3.1 g, 9.0 mmol, 1.0 eq) dissolved in 125 ml of a dichloromethane/methanol 2:1 mixture, under argon, is stirred at room temperature in the dark for 3 hours. The reaction mixture is treated by slowly adding, at 0° C., a 1M aqueous solution of hydrochloric acid and then its pH is adjusted to pH 5 by adding aqueous solution of sodium hydrogen carbonate. After decanting, the aqueous phase is extracted with a dichloromethane/methanol mixture (3 times). The collected organic phases are washed with saturated aqueous solution of sodium chloride, dried over magnesium sulfate, filtered and concentrated at reduced pressure. A portion of the crude reaction product (m=1.7 g, corresponding to 4.02 mmol theoretical of compound IV-3) is used directly in the next step. Another portion of the crude product (1 g) is purified by silica column chromatography using a heptane/dichloromethane gradient (95:5 to 0:100) to give compound IV-3 (732 mg, 1.72 mmol, 73% yield based on 2.33 mmol of starting product).

A mixture of unpurified compound IV-3 (4.02 mmol, 1.0 eq) and K₂CO₃ (2.8 g, 20.10 mmol, 5.0 eq) dissolved in acetonitrile (80 ml) is cooled to 0° C. for 30 min. MOMCl (1.62 ml, 21.30 mmol, 5.3 eq) is added dropwise, gradually over a period of 28 h, and then 39 ml of water is added. The pH of the solution is adjusted to pH 6 by adding a 2M 5 aqueous solution of hydrochloric acid HCl. After decanting, the aqueous phase is extracted 4 times with ethyl acetate. The combined organic phases are washed with saturated aqueous solution of sodium chloride, dried over magnesium sulfate, filtered and concentrated at reduced pressure. The crude product is purified by silica column chromatography using a heptane/EtOAc gradient (95:5 to 0:100) to give compound IV-4 in the form of a pale yellow solid (1.4 g, 3.08 mmol, 77%).

### Geranylation of compound IV-4 (step (D))

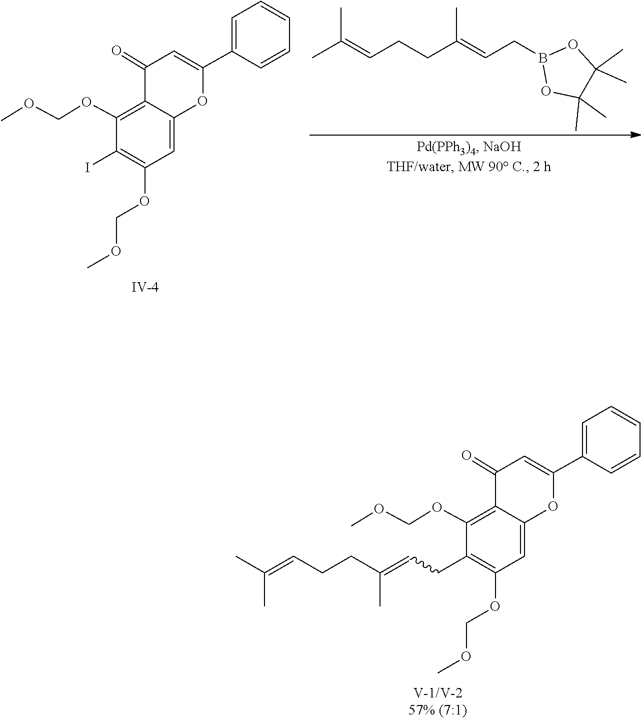

Sodium hydroxide (NaOH: 171 mg, 4.27 mmol, 2.5 eq) and tetrakis(triphenylphosphine) palladium(0) (Pd(PPh3)4:27 mg, 0.02 mmol, 0.01 eq) are added to a solution of compound IV-4 (800 mg, 1.71 mmol, 1.0 eq) and geranylboronic ester (813 mg, 3.08 mmol, 1.8 eq) in 8 ml of a mixture (1:1) of degassed THF/water. The reaction mixture is stirred at 90° C. for 2 hours in a microwave oven and then filtered on Celite®. After decanting, the aqueous phase is extracted with ethyl acetate three times. The combined organic phases are washed with saturated aqueous solution of sodium chloride, dried over magnesium sulfate, filtered and concentrated at reduced pressure. The crude product is purified by silica column chromatography using a heptane/EtOAc gradient (95:5 to 0:100) to give compounds V-1 and V-2, as an inseparable mixture (7:1), in the form of pale yellow oil (466 mg, 0.97 mmol, 57%).

### Epoxidation of the inseparable mixture V-1 and V-2 (step (F))

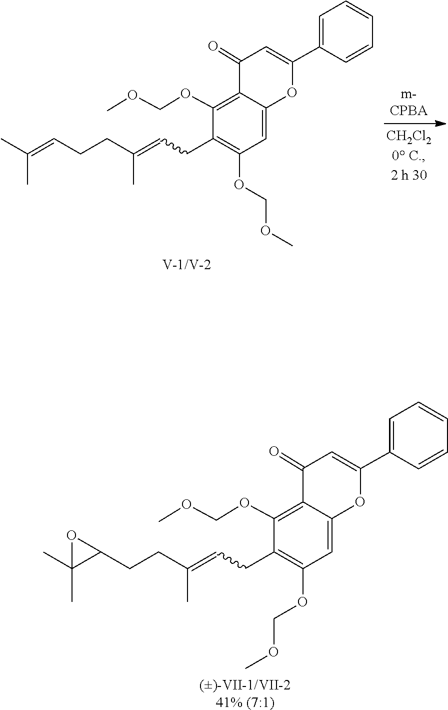

Metachloroperbenzoic acid (m-CPBA: 631 mg, 3.66 mmol, 3.5 eq) is added to the mixture of compounds V-1 and V-2 (500 mg, 1.05 mmol, 1.0 eq) dissolved in 5 ml of dichloromethane cooled to 0° C. The reaction mixture is stirred for 18 h at the same temperature and then 10 ml of additional dichloromethane is added. The suspension is filtered on Celite® and then the filtrate is washed with a 10% aqueous solution of sodium hydroxide. The aqueous phase is decanted and extracted with dichloromethane 3 times.

The combined organic phases are washed with water, dried over magnesium sulfate, filtered and concentrated. The crude product is purified by silica column chromatography using a heptane/EtOAc gradient (95:5 to 0:100) to give a mixture of the inseparable racemic compounds (±)-VII-1 and (±)-VII-2 (180 mg, 0.36 mmol, 35%) in the form of orange oil.

### Cyclization of the mixture of compounds (±)-VII-1 and (±)-VII-2 (step (G) (i))

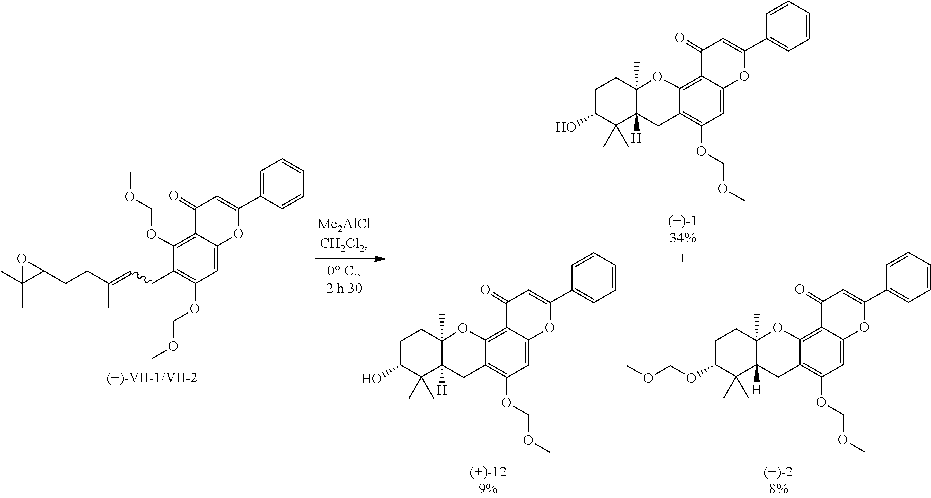

The mixture of the inseparable racemic compounds (±)-VII-1 and (±)-VII-2 (170 mg, 0.34 mmol, 1.0 eq) is dissolved in 4 ml of anhydrous dichloromethane, placed under argon and then cooled to 0° C. A 1M solution in hexane of dimethyl aluminum chloride is added (0.62 ml, 0.62 mmol, 2.0 eq), and the reaction mixture is stirred for 2.5 h at 0° C. A 2M aqueous solution of hydrochloric acid (0.4 ml) and then magnesium sulfate are added successively. The reaction mixture is filtered and then the filtrate is concentrated at reduced pressure. The crude product is purified by silica column chromatography using a heptane/EtOAc gradient (95:5 to 0:100) to give the pure racemic compounds (±)-1 (52 mg, 0.12 mmol, 34%), (±)-12 (14 mg, 0.03 mmol, 9%) and (±)-2 (18 mg, 0.03 mmol, 8%) in the form of yellow oils.

### Formation of the compound (±)-3 (step (G) (ii))

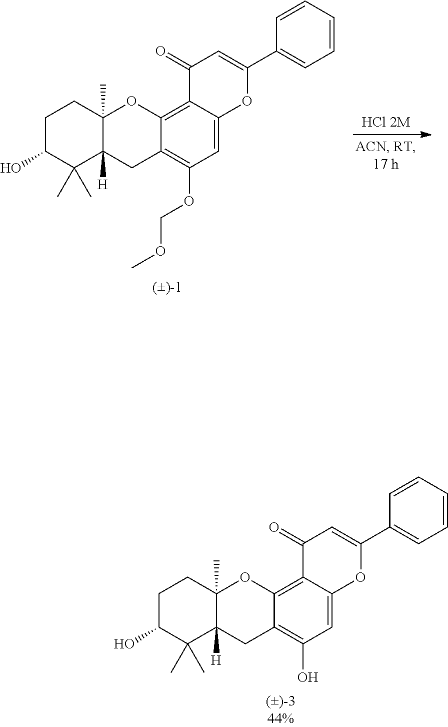 [SMILES: COCOc1cc2oc(-c3ccccc3)cc(=O)c2c2c1C[C@@H]1C(C)(C)[C@H](O)CC[C@@]1(C)O2 ; CC1(C)[C@H](O)CC[C@@]2(C)Oc3c(c(O)cc4oc(-c5ccccc5)cc(=O)c34)C[C@H]12]

A 2M aqueous solution of hydrochloric acid (0.68 mmol, 8.0 eq) is added to a solution of the compound (1)-1 (38 mg, 0.08 mmol, 1.0 eq) in 0.6 ml of acetonitrile. The reaction mixture is stirred for 17 h at room temperature and then filtered. The yellow solid obtained is washed with cold acetonitrile and water to give the pure compound (±)-3 (15 mg, 0.04 mmol, 44%).

### Example 2: Preparation of the Compounds of Formula (I) Designated 10, 11 and 15 According to the Invention

### Protection of kaempferol II-3 (step (B))

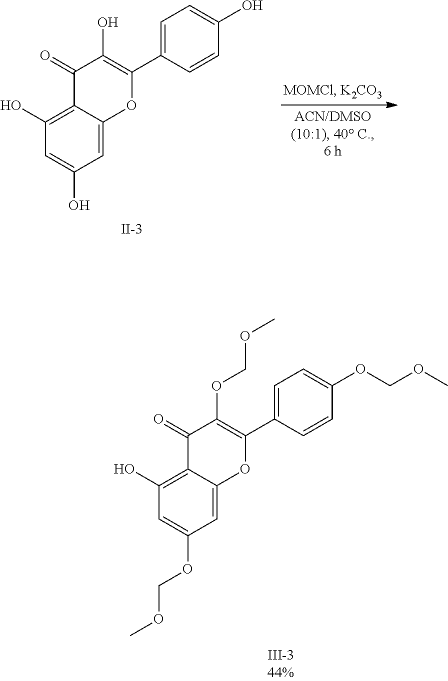 [SMILES: O=c1c(O)c(-c2ccc(O)cc2)oc2cc(O)cc(O)c12 ; COCOc1ccc(-c2oc3cc(OCOC)cc(O)c3c(=O)c2OCOC)cc1]

A mixture of kaempferol II-3 (1.80 g, 6.29 mmol, 1.0 eq) and potassium carbonate (K₂CO₃: 6.08 g, 44.02 mmol, 7.0 eq) dissolved in 39 ml of an acetonitrile/DMSO 10:1 mixture is placed under an argon atmosphere and heated at 40° C. for 1 hour. Methyl chloromethyl ether (MOMCl: 2.11 ml, 31.44 mmol, 5.0 eq) is added very gradually over a period of 1 hour, and then 23 ml of 2M aqueous solution of hydrochloric acid is added. After decanting, the aqueous phase is extracted with ethyl acetate three times. The collected organic phases are washed with saturated aqueous solution of sodium chloride, and then dried over magnesium sulfate, filtered and concentrated at reduced pressure. The crude product is purified by silica column chromatography using a heptane/EtOAc/1% AcOH ternary mixture to give the pure compound III-3 in the form of a pale yellow solid (1.16 g, 2.77 mmol, 44%).

### Iodation (step (C)) and geranylation of III[-3 (step (D))

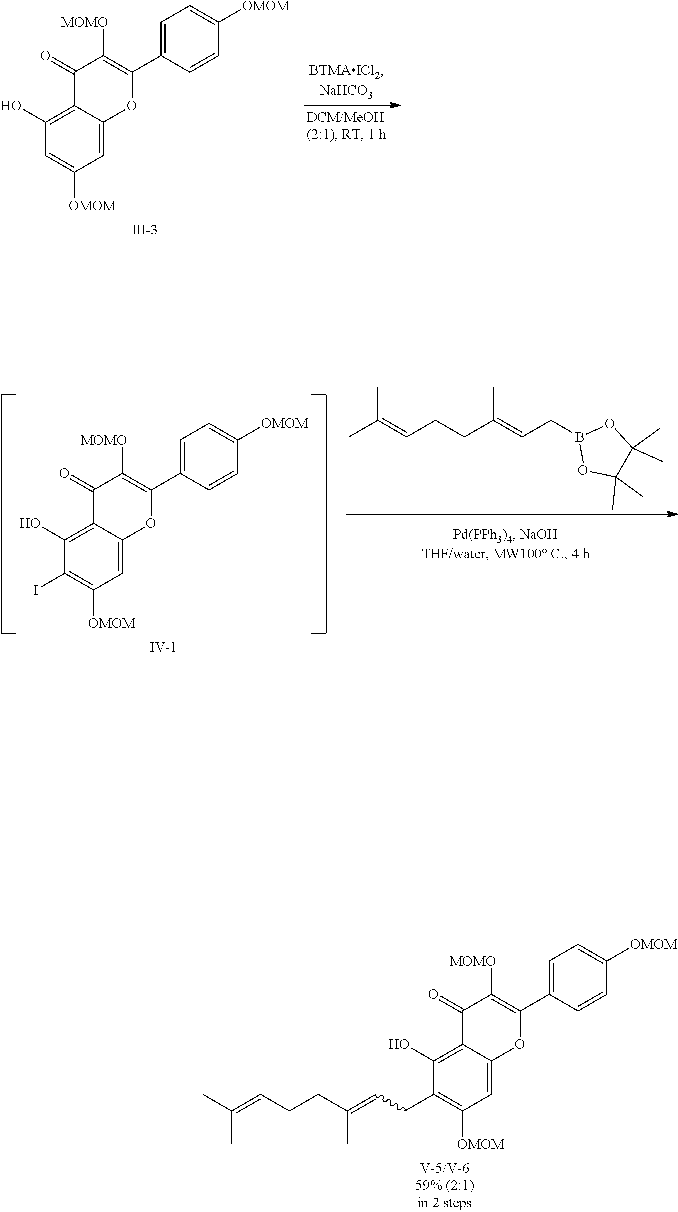

A mixture of compound III-3 (314 mg, 0.75 mmol, 1.0 eq), sodium hydrogen carbonate (NaHCO₃: 315 mg, 3.75 mmol, 5.0 eq) and benzyltrimethylammonium dichloroiodate (BTMA-ICl2:256 mg, 0.74 mmol, 0.98 eq) in 6 ml of a dichloromethane/methanol 2:1 mixture under an argon atmosphere is stirred at room temperature in the dark for 1 hour. The reaction mixture is treated by slowly adding, at 0° C., a 1M aqueous solution of hydrochloric acid and then its pH is adjusted to pH 5 by adding aqueous solution of sodium hydrogen carbonate. After decanting, the aqueous phase is extracted with a dichloromethane/methanol 2:1 mixture (3 times). The collected organic phases are washed with saturated aqueous solution of sodium chloride, dried over magnesium sulfate and concentrated at reduced pressure to give compound IV-1 (400 mg, 98% yield based on initial 0.75 mmol) which is used directly in the next step (step (D)).

Sodium hydroxide (NaOH: 59 mg, 1.47 mnol, 2.0 eq) and tetrakis(triphenylphosphine) palladium(0) (Pd(PPh₃)₄: 23 mg, 0.01 mmol, 0.02 eq) are added to a solution of compound IV-1 (0.74 mmol, 1.0 eq) and geranylboronic ester (291 mg, 1.10 mmol, 1.5 eq) in 9 ml of a 1:1 mixture of degassed THF/water. The reaction mixture is then stirred at 100° C. for 4 hours in a microwave oven and then filtered on Celite®. After decanting, the aqueous phase is extracted with ethyl acetate three times. The combined organic phases are washed with saturated aqueous solution of sodium chloride, dried over magnesium sulfate, filtered and concentrated. The crude product is purified by silica column chromatography using a heptane/EtOAc/1% AcOH ternary mixture to give compounds V-5 and V-6 as an inseparable 2:1 mixture in the form of pale yellow oil (240 mg, 0.43 mmol, 59% in 2 steps starting from compound III-3).

### Epoxidation (step (F)) of the mixture of compounds V-5 and V-6 via dihydroxylation (step (E))

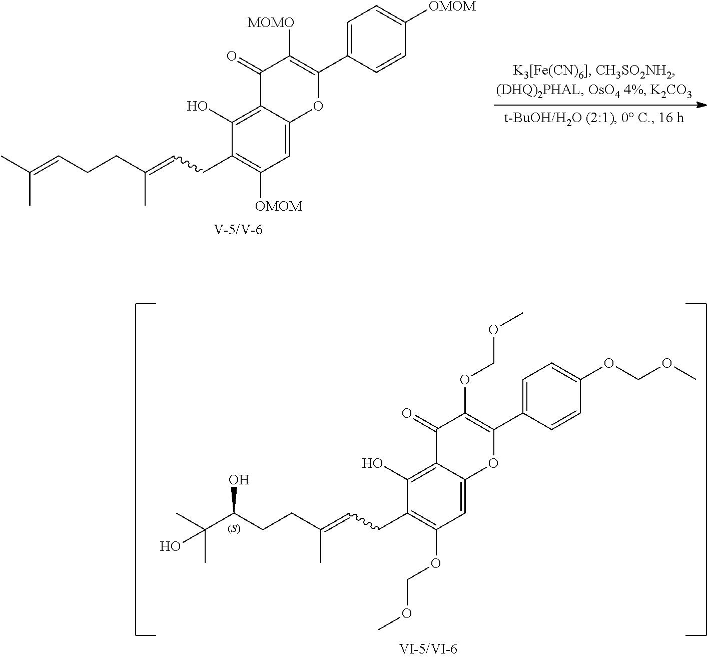

Potassium carbonate (K₂CO₃: 219 mg, 2.45 mmol, 4.0 eq), potassium ferricyanide (K₃Fe(CN)₆: 366 mg, 1.11 mmol, 2.8 eq), hydroquinine 1,4-phthalazinediyl diether ((DHQ)₂PHAL: 12 mg, 0.02 mmol, 0.04 eq), a 4% aqueous solution of osmium tetroxide (OsO₄: 0.13 ml 4% in water, 0.02 mmol, 0.04 eq) and methanesulfonamide (CH₃SO₂NH₂: 49 mg, 0.52 mmol, 1.3 eq) are added successively to a solution of compounds V-5 and V-6 (220 mg, 0.40 mmol, 1.0 eq) in 5 ml of a t-BuOH/water 2:1 mixture. The reaction mixture is stirred at 0° C. for 16 hours, and then sodium sulfite (Na₂SO₃: 140 mg, 1.11 mmol, 2.8 eq) is added. The aqueous phase is decanted and extracted with ethyl acetate three times.

The combined organic phases are washed with saturated aqueous solution of sodium chloride, dried over magnesium sulfate, filtered and concentrated at reduced pressure. The intermediate diols VI-5 and VI-6 in 5:2 mixture are used directly in the next step (step (F)).

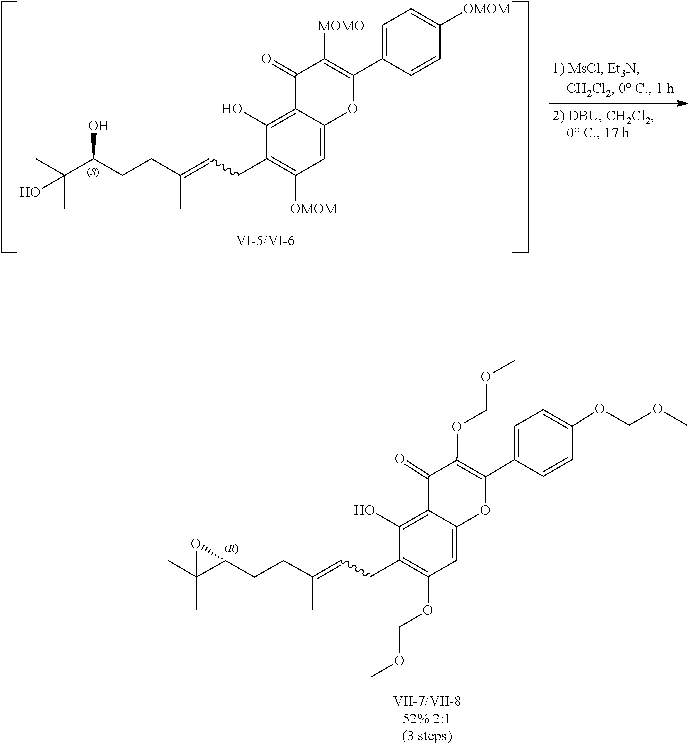

Freshly distilled triethylamine (Et₃N: 0.25 ml, 1.84 mmol, 4.6 eq) is added at 0° C. to a solution of the mixture of diols VI-5 and VI-6 (0.40 mmol, 1.0 eq) in 3 ml of anhydrous dichloromethane under an argon atmosphere. The mixture is stirred at 0° C. for 30 minutes, then mesyl chloride (MsCl: 0.07 ml, 0.92 mmol, 2.3 eq) is added and stirring is maintained for 1 hour. On completion of the mesylation reaction (monitored by TLC), 1,8-diazabicyclo[5.4.0]undec-7-ene (DBU: 0.34 ml, 2.29 mmol, 5.7 eq) is added. The reaction mixture is stirred for 17 hours at room temperature, and then a saturated aqueous solution of ammonium chloride is added. The aqueous phase is decanted and extracted with ethyl acetate three times. The combined organic phases are washed with water, dried over magnesium sulfate, filtered and concentrated. The product is purified by silica column chromatography using a heptane/EtOAc/10% AcOH ternary mixture, leading to the inseparable compounds VII-7 and VII-8 in 2:1 ratio in the form of pale yellow oil (117 mg, 0.21 mmol, 52% in 3 steps starting from the mixture of compounds V-5 and V-6).

### Cyclization of the mixture of compounds VII-7 and VII-8 (step (G) (i))

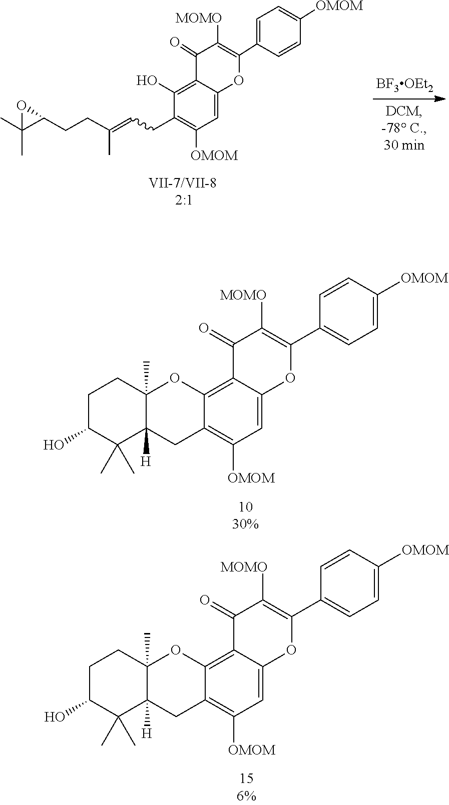

The mixture of epoxides VII-7 and VII-8 (89 mg, 0.16 mmol, 1.0 eq) dissolved in 8 ml of anhydrous dichloromethane under argon is cooled to −78° C. A 10% solution of boron trifluoride ether in anhydrous dichloromethane (0.29 ml, 0.23 mmol, 1.5 eq) is added dropwise. The reaction mixture is stirred for 30 min at −78° C. before adding 1 ml of water. The aqueous phase is decanted and extracted with dichloromethane 3 times. The combined organic phases are washed with water, dried over magnesium sulfate and concentrated at reduced pressure. The crude reaction product is purified on a preparative silica plate, using a heptane/EtOAc/acetic acid ternary mixture (2/8/0.1), to give the pure compounds 10 (27 mg, 0.05 mmol, 30%) and 15 (5 mg, 0.009 mmol, 6%) in the form of yellow oils.

### Production of compound 11 (step (G) (ii))

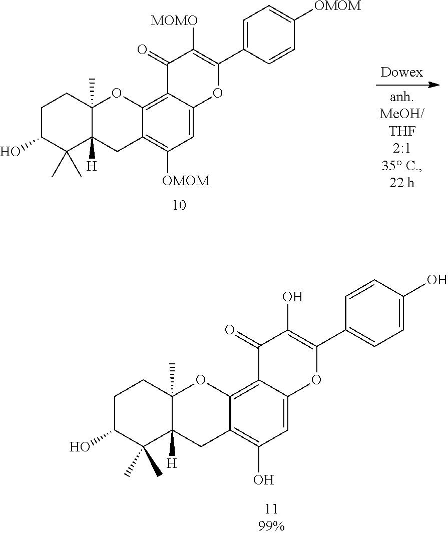

A suspension of compound 10 (8.5 mg, 0.01 mmol, 1 eq) in 0.3 ml of a 2:1 mixture of anhydrous methanol/anhydrous THF under argon is heated to 35° C. An acid ion exchange resin (Dowex® 50WX8:47 mg, 0.2 mmol, 15 eq) is added. The reaction mixture is stirred for 22 h at 35° C. and then filtered and concentrated at reduced pressure. The product is purified by silica column chromatography using a heptane/EtOAc/1% AcOH ternary mixture, and then an EtOAc/MeOH mixture, leading to the expected pure compound 11 (6.5 mg, 0.01 mmol, 99%) in the form of a yellow solid.

### Example 3: Preparation of the Compounds of Formula (I) Designated 6, 7 and 14 According to the invention

### Protection of chrysin II-1 (step (B)

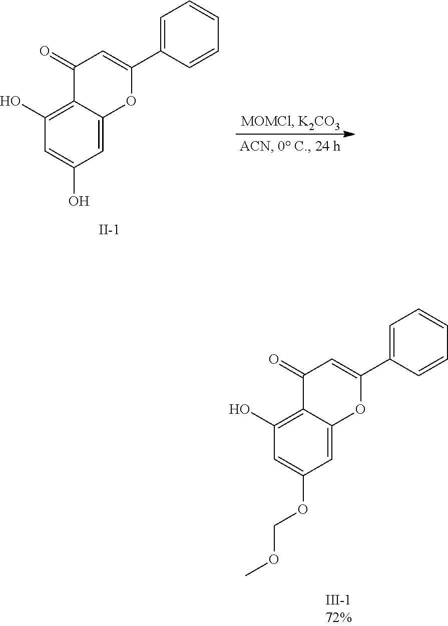

A mixture of chrysin II-1 (5.0 g, 19.7 mmol, 1.0 eq) and potassium carbonate (K₂CO₃: 13.6 g, 98.3 mmol, 5.0 eq) dissolved in 197 ml of acetonitrile is placed under an argon atmosphere and cooled to 0° C. for 30 min. Methyl chloromethyl ether (MOMCl: 2.6 ml, 34.8 mmol, 1.8 eq) is added dropwise, at 0° C., over a period of 1 hour. The mixture is stirred at the same temperature for 2 hours and then 197 ml of water is added. After decanting, the aqueous phase is extracted with ethyl acetate three times. The collected organic phases are washed with saturated aqueous solution of sodium chloride, and then dried over magnesium sulfate, filtered and concentrated at reduced pressure. The crude product is purified by silica column chromatography using a heptane/EtOAc gradient (95:5 to 0:100) to give the pure compound III-1 (4.2 g, 14.1 mmol, 72%) in the form of a yellow solid.

### Iodation of compound III-1 (step (C)) and geranylation (step (D))

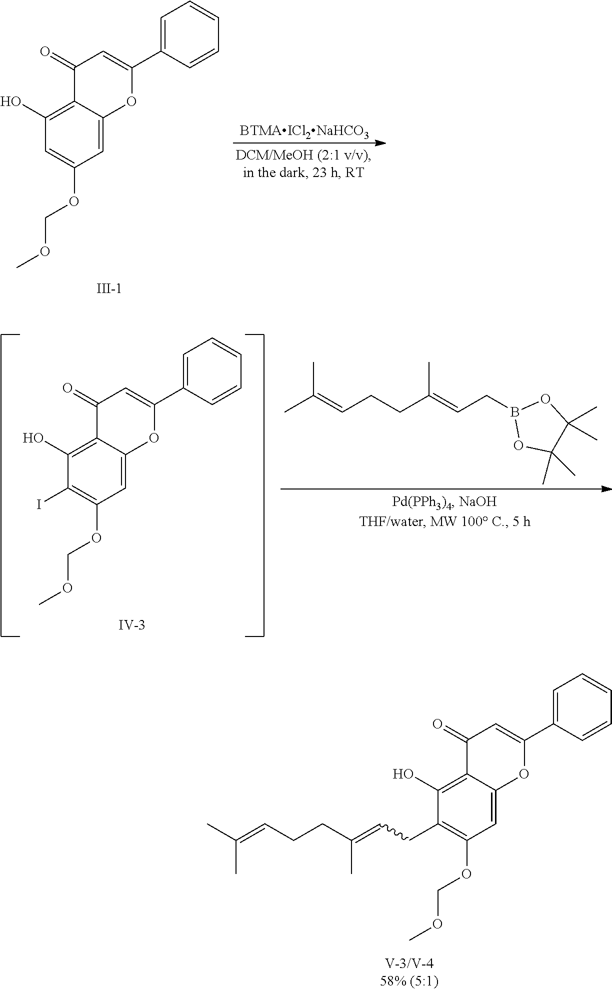

A mixture of compound III-1 (1.4 g, 4.7 mmol, 1.0 eq), sodium hydrogen carbonate (NaHCO₃: 3.9 g, 46.9 mmol, 10.0 eq) and benzyltrimethylammonium dichloroiodate (BTMA-ICl2:1.6 g, 4.7 mmol, 1.0 eq) dissolved in 66 ml of a dichloromethane/methanol 2:1 mixture, under argon, is stirred at room temperature in the dark for 23 hours. The reaction mixture is treated by slowly adding, at 0° C., a 1M aqueous solution of hydrochloric acid and then its pH is adjusted to pH 5 by adding aqueous solution of sodium hydrogen carbonate. After decanting, the aqueous phase is extracted with a dichloromethane/methanol mixture (4 times). The collected organic phases are washed with saturated aqueous solution of sodium chloride, dried over magnesium sulfate, filtered and concentrated at reduced pressure. A portion of the crude reaction product (m=1.3 g, corresponding to 3.06 mmol theoretical of compound IV-3) is used directly in the next step.

Sodium hydroxide (NaOH: 306 mg, 7.66 mmol, 2.5 eq) and tetrakis(triphenylphosphine) palladium(0) (Pd(PPh₃)4:48 mg, 0.03 mmol, 0.01 eq) are added to a solution of compound IV-3 (3.06 mmol, 1.0 eq) and geranylboronic ester (1215 mg, 4.60 mmol, 1.5 eq) in 16 ml of a 1:1 mixture of degassed THF/water. The reaction mixture is then stirred at 100° C. for 5 hours in a microwave oven and then filtered on Celite*. After decanting, the aqueous phase is extracted with ethyl acetate five times. The combined organic phases are washed with saturated aqueous solution of sodium chloride, dried over magnesium sulfate, filtered and concentrated. The crude product is purified by silica column chromatography using a heptane/EtOAc ternary mixture to give the compounds V-3 and V-4 as 5:1 inseparable mixture in the form of pale yellow oil (774 mg, 1.78 mmol, 59% in 2 steps starting from compound III-3).

### Dihydroxylation of the mixture of compounds V-3 and V-4 (step (E))

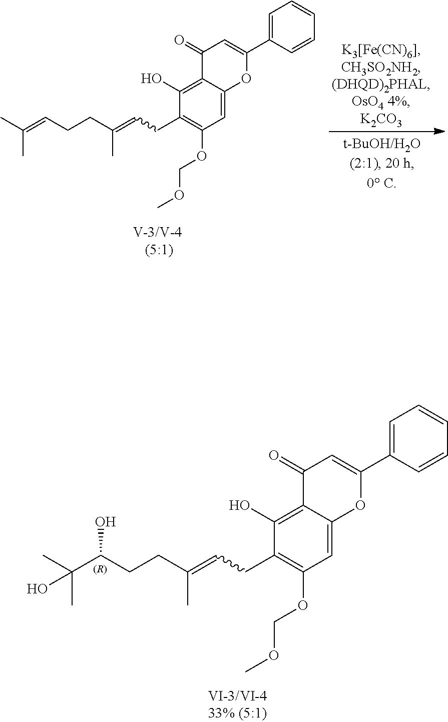

Potassium carbonate (K₂CO₃: 623 mg, 4.51 mmol, 4.0 eq), potassium ferricyanide (K₃Fe(CN)₆: 743 mg, 2.26 mmol, 2.0 eq), hydroquinidine 1,4-phthalazinediyl diether ((DHQD)₂PHAL: 35 mg, 0.05 mmol, 0.04 eq), a 4% aqueous solution of osmium tetroxide (OsO₄: 0.29 ml 4% in water, 0.05 mmol, 0.04 eq) and methanesulfonamide (CH₃SO₂NH₂: 139 mg, 1.45 mmol, 1.3 eq) are added successively to a solution of compounds V-3 and V-4 (490 mg, 1.13 mmol, 1.0 eq) in 5 ml of a t-BuOH/water 2:1 mixture. The reaction mixture is stirred at 0° C. for 20 hours, and then sodium sulfite (Na₂SO₃: 284 mg, 2.26 mmol, 2 eq) is added. The aqueous phase is decanted and extracted with ethyl acetate three times. The combined organic phases are washed with saturated aqueous solution of sodium chloride, dried over magnesium sulfate, filtered and concentrated at reduced pressure. The product is purified by silica column chromatography using a heptane/EtOAc/1% AcOH ternary mixture, leading to the inseparable compounds VI-3 and VI-4 in 5:1 ratio in the form of pale yellow oil (176 mg, 0.38 mmol, 33%).

### Formation of compounds VII-5 and VII-6 (step (F))

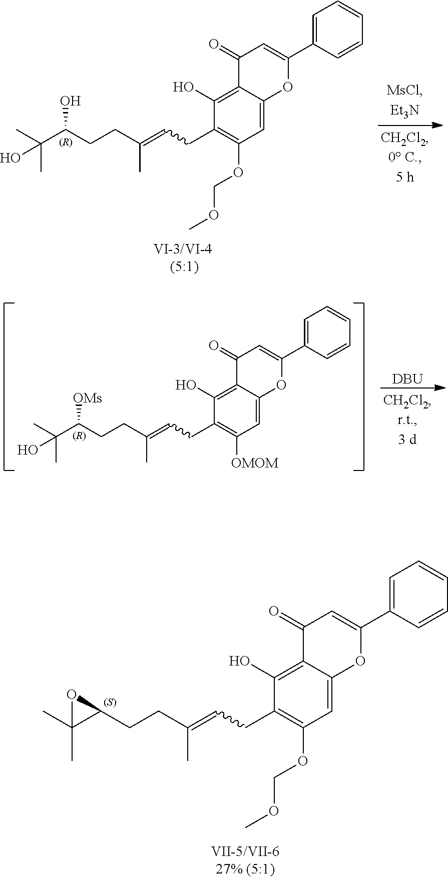

Freshly distilled triethylamine (Et3N: 0.50 ml, 3.71 mmol, 10.0 eq) is added at 0° C. to a solution of the mixture of diols VI-3 and VI-4 (0.37 mmol, 1.0 eq) in 2 ml of anhydrous dichloromethane under an argon atmosphere. The mixture is stirred at 0° C. for 30 minutes, then mesyl chloride (MsCl: 0.12 ml, 1.49 mmol, 4.0 eq) is added and stirring is maintained for 1 hour. On completion of the mesylation reaction (monitored by TLC), 1,8-diazabicyclo[5.4.0]undec-7-ene (DBU: 0.28 ml, 1.86 mmol, 5.0 eq) is added. The reaction mixture is stirred for 3 days at room temperature, and then a saturated aqueous solution of ammonium chloride is added. The aqueous phase is decanted and extracted with ethyl acetate three times. The combined organic phases are washed with water, dried over magnesium sulfate, filtered and concentrated. The product is purified by silica column chromatography using a heptane/EtOAc/10% AcOH ternary mixture, leading to the inseparable compounds VII-5 and VII-6 in 5:1 ratio in the form of pale yellow oil (44 mg, 0.10 mmol, 27%).

### Cyclization of the mixture of compounds VII-5 and VII-6 (step (G) (i))

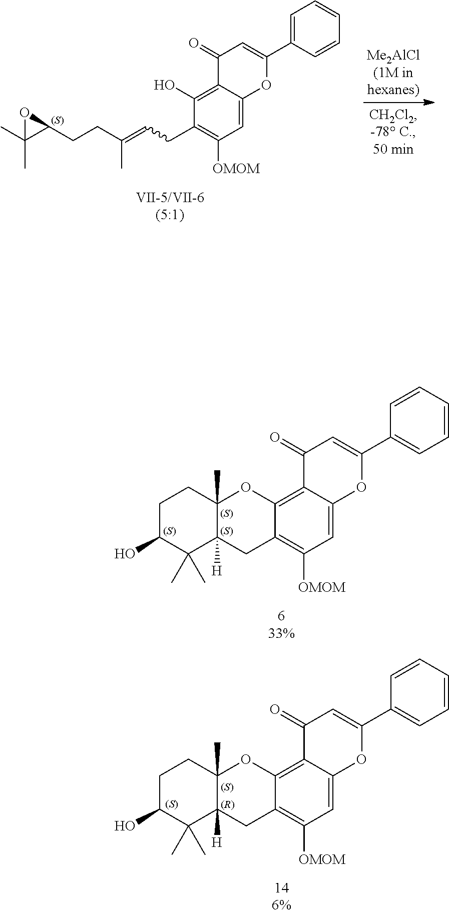

The mixture of epoxides VII-5 and VII-6 (33 mg, 0.07 mmol, 1.0 eq) dissolved in 1.5 ml of anhydrous dichloromethane under argon is cooled to −78° C. A 1M solution in hexane of dimethyl aluminum chloride is added (0.39 ml, 0.39 mmol, 5.3 eq), and the reaction mixture is stirred for 50 min at −78° C. A 2M aqueous solution of hydrochloric acid (0.3 ml) and then magnesium sulfate are added successively. The reaction mixture is filtered and then the filtrate is concentrated at reduced pressure. The crude reaction product is purified on a preparative silica plate, using a heptane/EtOAc/acetic acid ternary mixture (3/7/0.1), to give the pure compounds 6 (11 mg, 0.02 mmol, 33%) and 14 (2 mg, 0.004 mmol, 6%) in the form of yellow oils.

### Production of compound 7 (step (G) (ii))

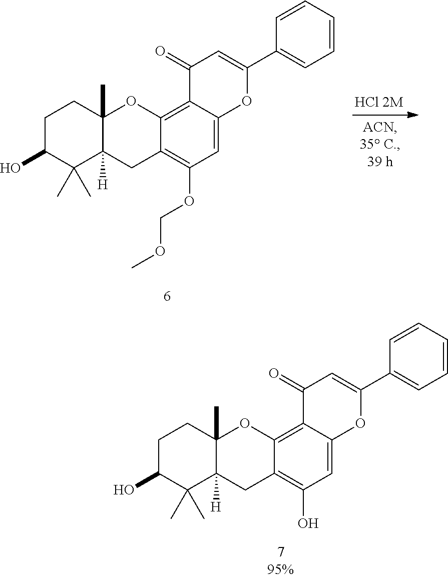 [SMILES: COCOc1cc2oc(-c3ccccc3)cc(=O)c2c2c1C[C@H]1C(C)(C)[C@@H](O)CC[C@]1(C)O2 ; CC1(C)[C@@H](O)CC[C@]2(C)Oc3c(c(O)cc4oc(-c5ccccc5)cc(=O)c34)C[C@@H]12]

A 2M aqueous solution of hydrochloric acid (0.47 mmol, 30.0 eq) is added to a solution of compound 6 (7.0 mg, 0.016 mmol, 1.0 eq) in 0.3 ml of acetonitrile. The reaction mixture is stirred for 39 h at 35° C. and then filtered. The yellow solid obtained is washed with cold acetonitrile and water, leading to the pure compound 7 (6.0 mg, 0.015 mmol, 95%).

### Example 4: Preparation of the Compounds of Formula (I) Designated 8, 9 and 18 According to the Invention

Protection of apigenin II-2 (step (B))

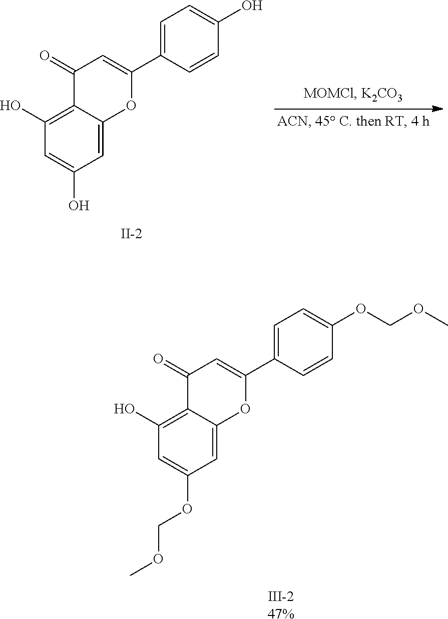 [SMILES: O=c1cc(-c2ccc(O)cc2)oc2cc(O)cc(O)c12 ; "II-2" ; COCOc1ccc(-c2cc(=O)c3c(O)cc(OCOC)cc3o2)cc1 ; "III-247%"]

A mixture of apigenin II-2 (1.0 g, 3.90 mmol, 1.0 eq) and potassium carbonate (K₂CO₃: 2.1 g, 15.39 mmol, 4.0 eq) in 39 ml of acetonitrile is placed under an argon atmosphere and heated at 45° C. for 1 hour. The reaction mixture is cooled to room temperature, and methyl chloromethyl ether (MOMCl: 0.49 ml, 7.31 mmol, 1.9 eq) is added very gradually over a period of 2.5 h and then 15 ml of a 2M aqueous solution of hydrochloric acid is added. After decanting, the aqueous phase is extracted with ethyl acetate three times. The collected organic phases are washed with saturated aqueous solution of sodium chloride, and then dried over magnesium sulfate, filtered and concentrated at reduced pressure. The crude product is purified by silica column chromatography using a heptane/EtOAc gradient (95:5 to 0:100) to give the pure compound III-2 in the form of a pale yellow solid (644 mg, 1.80 mmol, 47%).

### Iodation (step (C)) and geranylation of III1-2 (step (D))

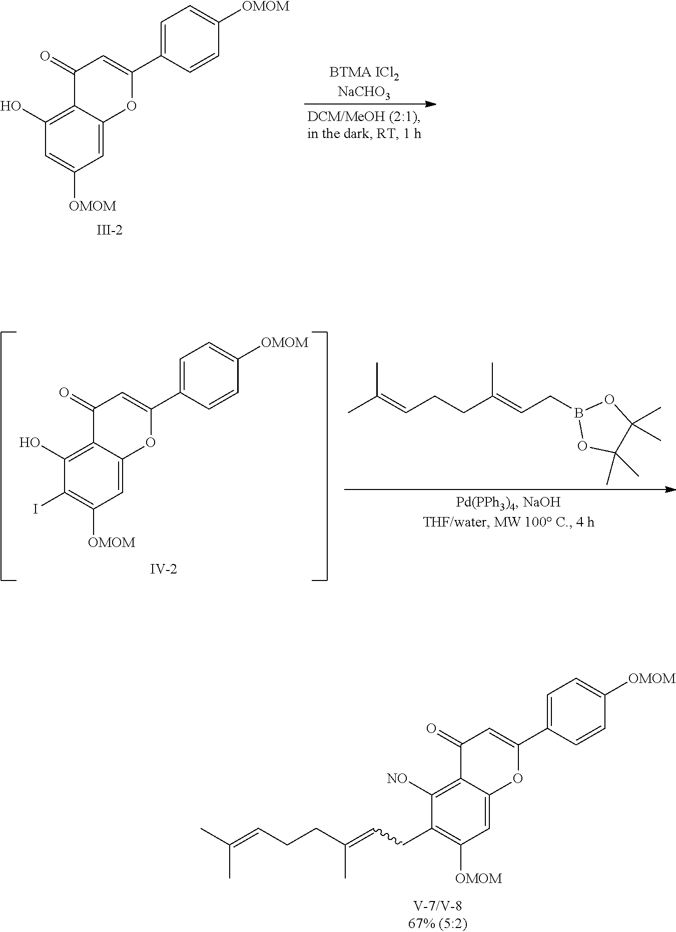

A mixture of compound III-2 (644 mg, 1.80 mmol, 1.0 eq), sodium hydrogen carbonate (NaHCO₃: 755 mg, 8.99 mmol, 5.0 eq) and benzyltrimethylammonium dichloroiodate (BTMA-ICl2:613 g, 1.76 mmol, 1.0 eq) in 13 ml of a dichloromethane/methanol 2:1 mixture under an argon atmosphere is stirred at room temperature in the dark for 1 hour. The reaction mixture is treated by slowly adding, at 0° C., a 1M aqueous solution of hydrochloric acid and then its pH is adjusted to pH 5 by adding aqueous solution of sodium hydrogen carbonate. After decanting, the aqueous phase is extracted with a dichloromethane/methanol 2:1 mixture (3 times). The collected organic phases are washed with saturated aqueous solution of sodium chloride, dried over magnesium sulfate and concentrated at reduced pressure to give compound IV-2 (857 mg, 98% on 1.80 mmol starting), which is used directly in the next step.

Sodium hydroxide (NaOH: 196 mg, 4.90 mmol, 2.5 eq) and tetrakis(triphenylphosphine) palladium(0) (Pd(PPh₃)4:46 mg, 0.03 mmol, 0.02 eq) are added to a solution of compound IV-2 (1.96 mmol, 1.0 eq) and geranylboronic ester (715 mg, 2.70 mmol, 1.4 eq) in 18 ml of a mixture (1:1) of degassed THF/water. The reaction mixture is stirred at 100° C. for 2 hours in a microwave oven and then filtered on Celite®. After decanting, the aqueous phase is extracted with ethyl acetate three times. The combined organic phases are washed with saturated aqueous solution of sodium chloride, dried over magnesium sulfate, filtered and concentrated. The crude product is purified by silica column chromatography using a heptane/EtOAc/1% AcOH ternary mixture to give compounds V-7 and V-8, as an inseparable 5:2 mixture in the form of pale yellow oil (650 mg, 1.31 mmol, 67% in 2 steps starting from compound III-2).

### Epoxidation (step (F)) of the mixture of compounds V-7 and V-8 via dihydroxylation (step (E))

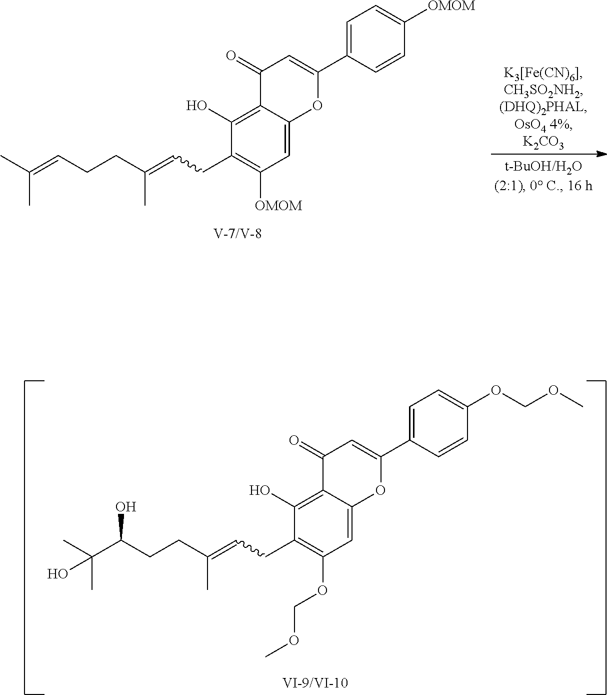

Potassium carbonate (K₂CO₃: 726 mg, 5.26 mmol, 4.0 eq), potassium ferricyanide (K₃Fe(CN)₆: 1.08 g, 3.29 mmol, 2.8 eq), hydroquinine 1,4-phthalazinediyl diether ((DHQ)₂PHAL: 41 mg, 0.05 mmol, 0.04 eq), a 4% aqueous solution of osmium tetroxide (OsO4:0.34 ml, 0.05 mmol, 0.04 eq) and methanesulfonamide (163 mg, 1.71 mmol, 1.3 eq) are added successively to a solution of compounds V-7 and V-8 (650 mg, 1.31 mmol, 1 eq) in 7 ml of a t-BuOH/water 2:1 mixture. The reaction mixture is stirred at 0° C. for 16 hours and then sodium sulfite (Na₂SO₃: 140 mg, 1.11 mmol, 2.8 eq) is added. The aqueous phase is decanted and extracted three times with EtOAc. The combined organic phases are washed with saturated aqueous solution of sodium chloride, dried over magnesium sulfate, filtered and concentrated at reduced pressure. The intermediate diols VI-9 and VI-10 in 5:2 mixture are used directly in the next step (step (F)).

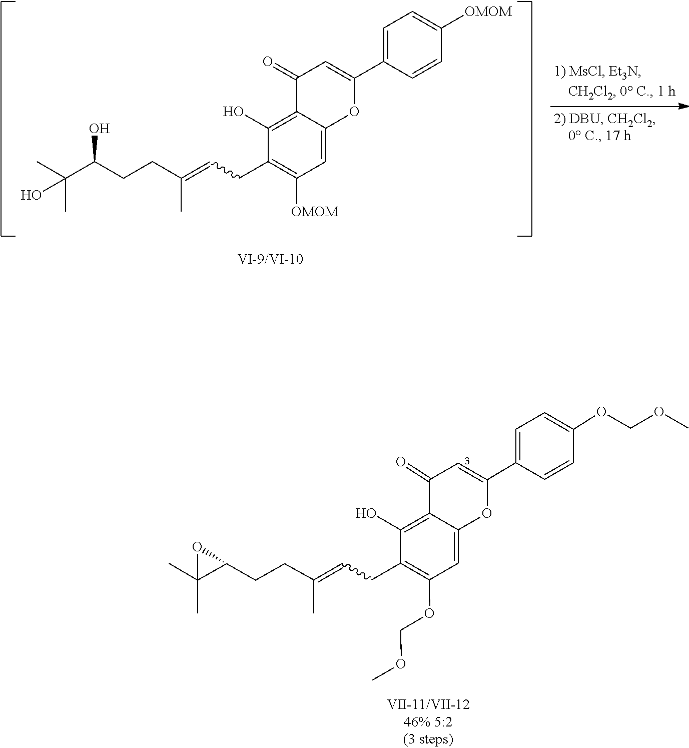

Freshly distilled triethylamine (Et₃N: 0.74 ml, 5.45 mmol, 4.1 eq.) is added, at 0° C., to a solution of the mixture of diols VI-9 and VI-10 (1.31 mmol, 1.0 eq.) in 8 ml of anhydrous dichloromethane under an argon atmosphere. The mixture is stirred at 0° C. for 30 minutes, then mesyl chloride (0.21 ml, 2.72 mmol, 2.1 eq) is added and stirring is maintained for 1 hour. On completion of the mesylation reaction (monitored by TLC), 1,8-diazabicyclo[5.4.0]undec-7-ene (DBU: 0.81 ml, 5.45 mmol, 4.0 eq) is added. The reaction mixture is stirred for 17 hours at room temperature, then a saturated aqueous solution of ammonium chloride is added and the whole is brought to room temperature. The aqueous phase is decanted and extracted with ethyl acetate three times. The combined organic phases are washed with water, dried over magnesium sulfate, filtered and concentrated. The product is purified by silica column chromatography using a heptane/EtOAc/1% AcOH ternary mixture, leading to the inseparable compounds VII-11 and VII-12 in 5:2 ratio in the form of pale yellow oil (310 mg, 0.61 mmol, 46% in 3 steps starting from the mixture of compounds V-7 and V-8).

### Cyclization of the mixture of compounds VII-11 and VII-12 (step (G) (i))

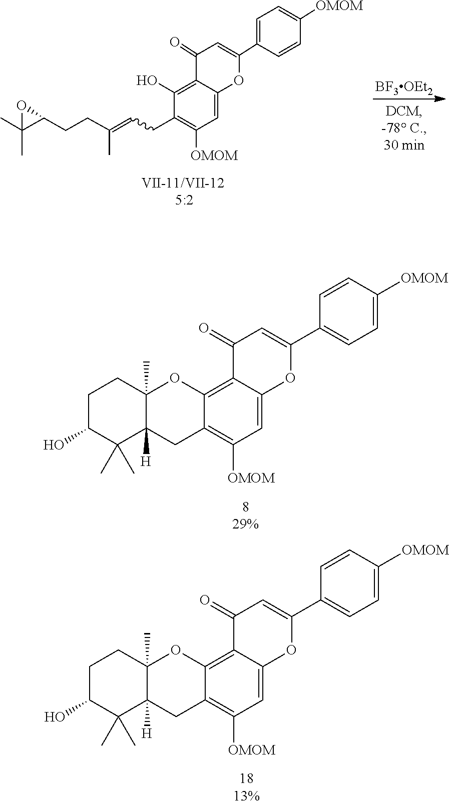

The mixture of epoxides VII-11 and VII-12 (31 mg, 0.06 mmol, 1.0 eq) dissolved in 3 ml of anhydrous dichloromethane under argon is cooled to −78° C. A 10% solution of boron trifluoride ether in anhydrous dichloromethane (0.15 ml, 0.12 mmol, 2.0 eq) is added dropwise. The reaction mixture is stirred for 30 min at −78° C. before adding 1 ml of water.

The aqueous phase is decanted and extracted with dichloromethane 3 times. The combined organic phases are washed with water, dried over magnesium sulfate and concentrated at reduced pressure. The crude reaction product is purified on a preparative silica plate, using a heptane/EtOAc/acetic acid ternary mixture (2/8/0.1), to give the pure compounds 8 (9 mg, 0.02 mmol, 29%) and 18 (4 mg, 0.01 mmol, 13%) in the form of yellow oils.

### Deprotection of 8 (step (G) (ii))

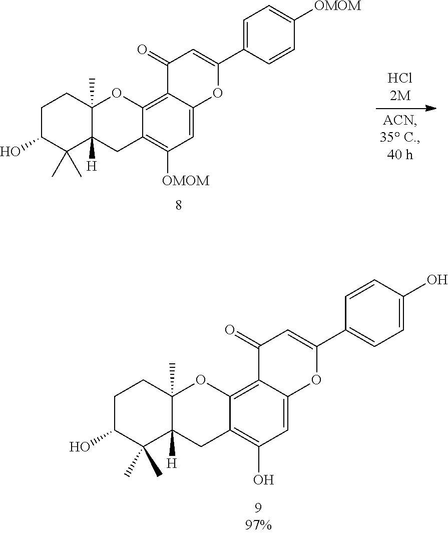

A suspension of compound 8 (9 mg, 0.02 mmol, 1.0 eq) in acetonitrile (0.2 ml) is heated at 35° C. until dissolved. A 2M aqueous solution of hydrochloric acid (0.39 ml, 0.78 mmol, 40 eq) is added, and the reaction mixture is stirred for 48 hours at 35° C. and then filtered. The solid obtained, corresponding to the expected product 9, is washed with cold acetone and water. The pure compound 9 is obtained in the form of a yellow solid (7.5 mg, 0.02 mmol, 97%).

### Example 5: Biological Activity

### Materials and Methods

Measurement of the inhibitory activity on OSBP is carried out by a robotized assay in the ORD domain (OSBP-Related Domain, in English) purified of the protein OSBP. The measurements are carried out according to a protocol using two preparations of liposomes:
The donor liposomes (A) contain a fluorescent sterol (DHE (dehydroergosterol));
The acceptor liposomes (B) contain a fluorescent lipid (dansyl PE (1,2-dioleoyl-sn-glycero-3-phosphoethanolamine-N-(5-dimethylamino-1-naphthalenesulfonyl) (ammonium salt)) whose excitation spectrum covers the emission spectrum of DHE.

Transport of DHE from liposomes A to liposomes B catalyzed by the ORD domain is accompanied by a FRET signal between DHE and Dansyl-PE. Based on this signal, the kinetics of transport can be measured in real time. This measurement of fluorescence is carried out on a microplate in a TECAN Infinite 1000 Pro instrument (temperature=37° C.). At the beginning, each measurement well contains liposomes B (130 μM), ORD domain (200 nM) and the test compound. At time t=5 min, liposomes A (130 μM) are added to start the exchange reaction. Each compound is tested in triplicate for final concentrations from 50 nM to 3 μM. The time constant (k) obtained for the kinetics in each case is then represented as a function of the concentration of the analog. From this representation, an inhibition constant is determined for each compound. The affinity constants Ki are classified as follows:
Very strong affinity Ki<10 nM
Good affinity Ki from 10 to 100 nM
Low affinity Ki from 100 to 2000 nM

The compounds, other than the prodrugs, having a Ki<100 nM are regarded as active on OSBP.

The lines U87-MG and A549 were obtained from the American Type Culture Collection (Rockville, MD, USA) and were cultured according to the supplier's instructions. The human glioblastoma cells U87-MG were cultured in Dulbecco minimum essential medium (DMEM) containing 10% of FCS and 1% of L-glutamine. The lung cancer cells A549 were cultured in RPMI1640 medium containing 10% of FCS and 1% of L-glutamine. The cell lines were maintained at 37° C. in a humidified atmosphere containing 5% CO₂. The products were tested at 10 concentrations in triplicate and the cellular viability was evaluated after 72 h of treatment using the CellTiter Glo assay (Promega), which allows the number of live cells to be measured by luminescence (quantification of ATP).

Thus, the cells were seeded in 96-well plates (2.5×10³ cells/well) each containing 90 μL of growth medium. After culture for 24 hours, the wells were supplemented with 10 μL of medium containing ten decreasing concentrations of the test compound dissolved in DMSO (less than 0.1% in each preparation). After incubation for 72 h, 100 μL of Cell Titer GLo reagent was added in the space of 15 min before quantifying the luminescence emitted using a microplate reader. The dose-response curve was analyzed using Graph Prism software and the activity of the molecules is expressed in the form of IC₅₀.

### Results

The compounds of formula (I) numbered 1 to 11 were evaluated on a glioblastoma cell line (U87) and a lung cancer cell line (A549). The results are presented in Table 11 hereunder.

TABLE 11

| | Affinity for the | Cytotoxicity | Cytotoxicity |
|---|---|---|---|
| Compound | target OSBP | on U87 | on A549 |
| No. | (Ki, nM) | (μM) | (μM) |
| 1 | Ki < 10 | 0.54 | 5.08 |
| 2 | 100 < Ki < 2 000 | >10.00 | >10.00 |
| 3 | Ki < 10 | 0.34 | 3.40 |
| 4 | 10 < Ki < 100 | 0.44 | n.t. |
| 5 | Ki < 10 | 0.28 | 1.38 |
| 6 | 100 < Ki < 2 000 | 4.31 | >10.00 |
| 7 | Ki < 10 | 1.89 | n.t. |
| 8 | 10 < Ki < 100 | 0.16 | n.t. |
| 9 | n.t. | 3.78 | n.t. |
| 10 | n.t. | 4.34 | n.t. |
| 11 | n.t. | 2.30 | 6.30 |

n.t.: “not tested”

### 15 Evaluation of Plasma Stability and Microsomal Stability

Tests of plasma stability and stability on hepatic microsomes were carried out on compounds 3, 9 and 10 according to the invention.

### Protocols

The compounds are placed in mouse plasma and mouse microsomes, and their stability is evaluated by monitoring by UPLC-MS/MS.

For plasma stability, the concentration tested is 2.5 μM and the incubation volume is 50 μL. The incubation times are 0, 15, 30, 45, 60 and 120 minutes. Protein precipitation is then performed on the samples, and then the latter are analyzed by UPLC-MS/MS. The percentage of the compound remaining as well as its elimination half-life are thus obtained. For the metabolic stability in microsomes, the concentration of test compound is 2.5 μM, and the incubation volume is 400 μL. The concentration of mouse microsomes is 0.5 mg/mL. The incubation times are 0, 15, 30 and 45 minutes. The cofactor used is NADPH. Two negative controls are carried out, one without microsomes and without cofactor at 0 and 45 minutes, and one without cofactor and with microsome at 0 and 45 minutes. A positive control is carried out in the presence of diphenhydramine. The samples are analyzed by LC-MS/MS. The intrinsic clearance and the half-life are thus obtained.

LC-MS/MS is carried out on a UPLC Acquity™ coupled to a XEVO TQ-S(WATERS).

Liquid chromatography is carried out on an Acquity UPLC column BEH 1.7 μm, 2.1×50 mm with a gradient of 4 minutes water+0.1% NH₃OH/acetonitrile 95:5 to 100:0. The column temperature is +50° C. and the temperature of the injector is +4° C. Acquisition in mass spectrometry is performed by MRM (Multiple Reaction Monitoring), with positive electrospray ionization.

### Results

The metabolic stability in mouse plasma at 120 minutes of the compound of formula (I) numbered 3 according to the invention is 99%, its metabolic stability in mouse microsomes is 88% and its clearance in microsomal medium is 152 μL/min/mg of protein.

The metabolic stability in mouse plasma at 120 minutes of the compound of formula (I) numbered 9 according to the invention is 100%, its metabolic stability in mouse microsomes is 100% and its clearance in microsomal medium is 26 μL/min/mg of protein.

The metabolic stability in mouse plasma at 120 minutes of the compound of formula (I) numbered 10 according to the invention is 90%, its metabolic stability in mouse microsomes is 99% and its clearance in microsomal medium is 52 μL/min/mg of protein.

It is clear from these results that the compounds of formula (I) according to the invention are stable in plasma and for the most part display good microsomal stability, more particularly for those whose clearance is below 50.

It is clear from all the bioassays presented above that the compounds of formula (I) according to the invention are useful as inhibitors of OSBP (oxysterol-binding protein) and may in particular be used as drugs, in particular for the prevention, inhibition and/or treatment of cancers, neurodegenerative diseases, dyslipidemia, hypercholesterolemia, and/or viral diseases.

1. Compound of formula (I), the pharmaceutically acceptable salts thereof, its hydrates, its solvates, as well as its tautomeric forms, stereoisomers, mixtures of stereoisomers, pure enantiomers and mixtures whether racemic or not 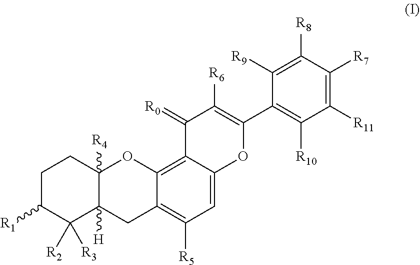
in which R₀ represents an oxygen atom or a group N—ORₐ,
R₁ represents a hydroxyl group or a protected hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group, a protected hydroxyl group, a group —O—P(═O)(OH)₂, a sugar radical, a hydrogen atom, a halogen atom, a —CF₃ group, a linear or branched C₁-C₄ alkyl group, a linear or branched C₂-C₄ alkenyl group or a linear or branched C₂-C₄ alkynyl group,
R₆ represents a hydrogen atom, a hydroxyl group, a protected hydroxyl group, a group —O—P(═O)(OH)₂, or a sugar radical,
R₇ represents a hydrogen atom, a hydroxyl group, a protected hydroxyl group, a halogen atom, a linear or branched C₁-C₄ alkyl group, a linear or branched C₂-C₄ alkenyl group, a linear or branched C₂-C₄ alkynyl group, a triazolyl group, a group —O—Rb, a group N(Rc)(Rd), a
group —C(═O)—N(Re)(Rf), —O—C(═O)—N(Rg)(Rh),
R₈ represents a hydrogen atom, a hydroxyl group or a protected hydroxyl group,
R₉ represents a hydrogen atom, a hydroxyl group or a protected hydroxyl group,
R₁₀ represents a hydrogen atom, a hydroxyl group or a protected hydroxyl group,
R₁₁ represents a hydrogen atom, a hydroxyl group or a protected hydroxyl group,
Ra, Rb, Rc, Rd, Re, Rf, Rg and Rh represent, independently of one another, a hydrogen atom or a linear or branched C₁-C₄ alkyl group, and the symbol [symbol] represents a single bond joining an asymmetric carbon atom to the group or atom in question, this bond being either in front of or behind the plane.

2. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which the protected hydroxyl group is selected from —O—CH₂—O—CH₃, a methoxy group, —O—C(═O)—CH₃, a tert-butyldimethylsilyloxy group, a benzyloxymethoxy group, a benzyloxy group, a trityloxy group, a para-methoxybenzyloxy group, a trimethylsilyloxy group, a tert-butyldiphenylsilyloxy group, a triisopropylsilyloxy group, and a pivaloyloxy group.

3. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which
R₀ represents an oxygen atom,
R₁ represents a hydroxyl group or a protected hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a protected hydroxyl group,
R₆ represents a hydrogen atom, a hydroxyl group or a protected hydroxyl group,
R₇ represents a hydrogen atom, a hydroxyl group or a protected hydroxyl group, and
R₈, R₉, R₁₀, and R₁₁ each represent a hydrogen atom.

4. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which
R₀ represents an oxygen atom
R₁ represents a hydroxyl group or a group —O—CH₂—O—CH₃,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a group —O—CH₂—O—CH₃,
R₆ represents a hydrogen atom, a hydroxyl group or a group —O—CH₂—O—CH₃,
R₇ represents a hydrogen atom, a hydroxyl group or a group —O—CH₂—O—CH₃, and
R₈, R₉, R₁₀, and R₁₁ each represent a hydrogen atom.

5. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which
R₀ represents an oxygen atom,
R₁ represents a hydroxyl group or a group —O—CH₂—O—CH₃,
R₂ represents a methyl group,
R₃ represents a methyl group,
R₄ represents a methyl group,
R₅ represents a hydroxyl group or a group —O—CH₂—O—CH₃,
R₆ represents a hydrogen atom, a hydroxyl group or a group —O—CH₂—O—CH₃,
R₇ represents a hydrogen atom, a hydroxyl group or a group —O—CH₂—O—CH₃, and
R₈, R₉, R₁₀, and R₁₁ each represent a hydrogen atom.

6. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which
R₀ represents an oxygen atom,
R₁ represents a hydroxyl group,
R₂ represents a linear or branched C₁-C₄ alkyl group,
R₃ represents a linear or branched C₁-C₄ alkyl group,
R₄ represents a linear or branched C₁-C₄ alkyl group,
R₅ represents a hydroxyl group or a protected hydroxyl group,
R₆ represents a hydrogen atom, and
R₇, R₈, R₉, R₁₀, and R₁₁ each represent a hydrogen atom.

7. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, in which
R₀ represents an oxygen atom,
R₁ represents a hydroxyl group,
R₂ represents a methyl group,
R₃ represents a methyl group,
R₄ represents a methyl group,
R₅ represents a hydroxyl group or a group —O—CH₂—O—CH₃,
R₆ represents a hydrogen atom, and
R₇, R₈, R₉, R₁₀, and R₁₁ each represent a hydrogen atom.

8. Compound of formula (I) according to claim 1, the pharmaceutically acceptable salts thereof, its hydrates, its solvates, as well as its tautomeric forms, stereoisomers, mixtures of stereoisomers, pure enantiomers and mixtures, whether or not racemic, selected from:
(7aR*,9R*,11aR*)-9-hydroxy-6-(methoxymethoxy)-8,8,11a-trimethyl-3-phenyl-7a,8,9,10,11,11a-hexahydro-1H,7H-pyrano[2,3-c]xanthen-1-one (+/− racemic mixture)
(7aR*,9R*,11aR*)-6,9-bis(methoxymethoxy)-8,8,11a-trimethyl-3-phenyl-7a,8,9,10,11,11a-hexahydro-1H,7H-pyrano[2,3-c]xanthen-1-one (+/− racemic mixture)
(7aR*,9R*,11aR*)-6,9-dihydroxy-8,8,11a-trimethyl-3-phenyl-7a,8,9,10,11,11a-hexahydro-1H,7H-pyrano[2,3-c]xanthen-1-one (+/− racemic mixture)
(7aR,9R,11aR)-9-hydroxy-6-(methoxymethoxy)-8,8,11a-trimethyl-3-phenyl-7a,8,9,10,11,11a-hexahydro-1H,7H-pyrano[2,3-c]xanthen-1-one (+)
(7aR,9R,11aR)-6,9-dihydroxy-8,8,11a-trimethyl-3-phenyl-7a,8,9,10,11,11a-hexahydro-1H,7H-pyrano[2,3-c]xanthen-1-one (+)
(7aS,9S,11aS)-9-hydroxy-6-(methoxymethoxy)-8,8,11a-trimethyl-3-phenyl-7a,8,9,10,11,11a-hexahydro-1H,7H-pyrano[2,3-c]xanthen-1-one (−)
(7aS,9S,11aS)-6,9-dihydroxy-8,8,11a-trimethyl-3-phenyl-7a,8,9,10,11,11a-hexahydro-1H,7H-pyrano[2,3-c]xanthen-1-one (−)
(7aR,9R,11aR)-9-hydroxy-6-(methoxymethoxy)-3-(4-(methoxymethoxy)phenyl)-8, 8,11a-trimethyl-7a,8,9,10,11,11a-hexahydro-1H,7H-pyrano[2,3-c]xanthen-1-one (+)
(7aR,9R,11aR)-6,9-dihydroxy-3-(4-hydroxyphenyl)-8,8,11a-trimethyl-7a,8,9,10,11,11a-hexahydro-1H,7H-pyrano[2,3-c]xanthen-1-one (+)
(7aR,9R,11aR)-9-hydroxy-2,6-bis(methoxymethoxy)-3-(4-(methoxymethoxy)phenyl)-8,8,11a-trimethyl-7a,8,9,10,11,11a-hexahydro-1H,7H-pyrano[2,3-c]xanthen-1-one (+)
(7aR,9R,11aR)-2,6,9-trihydroxy-3-(4-hydroxyphenyl)-8,8,11a-trimethyl-7a,8,9,10,11,11a-hexahydro-1H,7H-pyrano[2,3-c]xanthen-1-one (+)
(7aS*,9R*,11aR*)-9-hydroxy-6-(methoxymethoxy)-8,8,1a-trimethyl-3-phenyl-7a,8,9,10,11,11a-hexahydro-1H,7H-pyrano[2,3-c]xanthen-1-one (+/− racemic mixture)
(7aS,9R,11aR)-9-hydroxy-6-(methoxymethoxy)-8,8,11a-trimethyl-3-phenyl-7a,8,9,10,11,11a-hexahydro-1H,7H-pyrano[2,3-c]xanthen-1-one (+)
(7aR,9S,11aS)-9-hydroxy-6-(methoxymethoxy)-8,8,11a-trimethyl-3-phenyl-7a,8,9,10,11,11a-hexahydro-1H,7H-pyrano[2,3-c]xanthen-1-one (−)
(7aS,9R,11aR)-9-hydroxy-2,6-bis(methoxymethoxy)-3-(4-(methoxymethoxy)phenyl)-8,8,11a-trimethyl-7a,8,9,10,11,11a-hexahydro-1H,7H-pyrano[2,3-c]xanthen-1-one (+)
(7aS,9S,11aS)-9-hydroxy-2,6-bis(methoxymethoxy)-3-(4-(methoxymethoxy)phenyl)-8,8,11a-trimethyl-7a,8,9,10,11,11a-hexahydro-1H,7H-pyrano[2,3-c]xanthen-1-one (−)
(7aS,9S,11aS)-2,6,9-trihydroxy-3-(4-hydroxyphenyl)-8,8,11a-trimethyl-7a,8,9,10,11,11a-hexahydro-1H,7H-pyrano[2,3-c]xanthen-1-one (−) and
(7aS,9R,11aR)-9-hydroxy-6-(methoxymethoxy)-3-(4-(methoxymethoxy)phenyl)-8,8,11a-trimethyl-7a,8,9,10,11,11a-hexahydro-1H,7H-pyrano[2,3-c]xanthen-1-one (+).

9. Method of preparation of the compound of formula (I), as well as the pharmaceutically acceptable salts thereof, its hydrates, its solvates, as well as its tautomeric forms, stereoisomers, mixtures of stereoisomers, pure enantiomers and mixtures, whether or not racemic, comprising at least the following steps:
(i) cyclization of a compound of formula (VII) 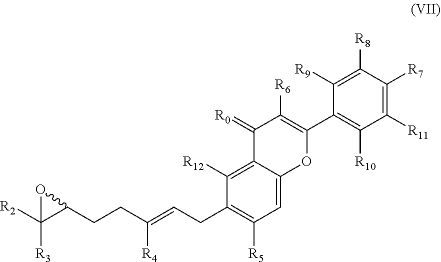
in which
R₀, and R₂ to R₁₁ are as defined according to claim 1 and
R₁₂ represents a hydroxyl group or a protected hydroxyl group, provided that at
least one of R₅ and R₁₂ is a protected hydroxyl group, and the symbol [symbol] represents a single bond joining an asymmetric carbon atom to the group or atom in question, this bond being either in front of or behind the plane, to obtain a compound of formula (I), and
(ii) Optionally deprotection of at least one hydroxyl group still protected by a protective group present in said compound obtained in step (i), to obtain a compound of formula (I).

10. Method according to claim 9, in which the compound of formula (VII) is prepared from a compound of formula (VI) 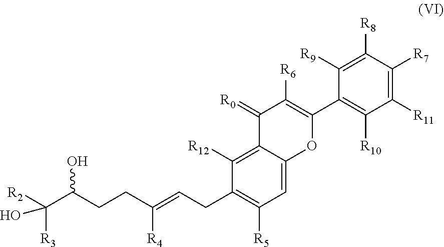
in which
R₀, R₂ to R₁₁ are as defined according to claim 1 and R₁₂ is a hydroxyl group or a protected hydroxyl group provided that at least one of R₅ and R₁₂ is a protected hydroxyl group, and the symbol [symbol] represents a single bond joining an asymmetric carbon atom to the group or atom in question, this bond being either in front of or behind the plane. or
in which the compound of formula (VII) is prepared from a compound of formula (V) 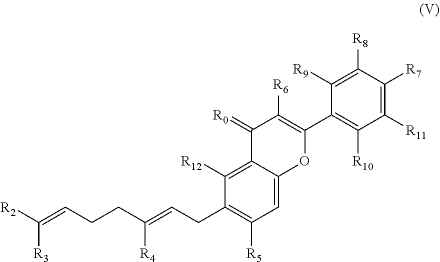
in which
R₀, R₂ to R₁₁ are as defined above according to and R₁₂ is as defined above, provided that at least one of R₅ and R₁₂ is a protected hydroxyl group.

11. Method according to claim 10, in which the compound of formula (VI) is prepared by dihydroxylation of a compound of formula (V) 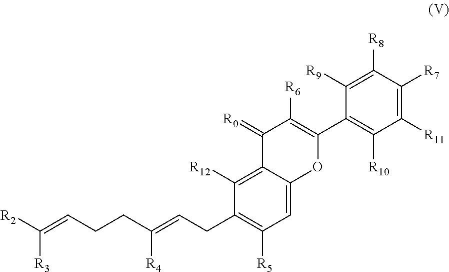
in which
R₀, R₂ to R₁₁ are as defined according to claim 1 and R₁₂ is a hydroxyl group or a protected hydroxyl group, provided that at least one of R₅ and R₁₂ is a protected hydroxyl group.

12. Method according to claim 10, in which the compound of formula (V) is prepared by geranylation of a compound of formula (IV) 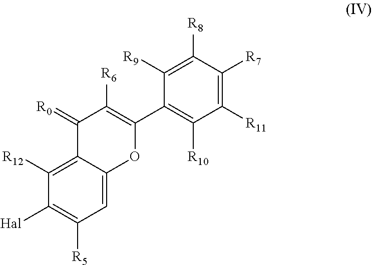
in which
R₀, R₅ to Rₙ are as defined according to claim 1 and R₁₂ is a hydroxyl group or a protected hydroxyl group; provided that at least one of R₅ and R₁₂ is a protected hydroxyl group, and Hal represents a halogen atom, using a compound of formula (VIII) 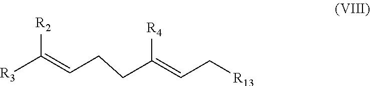
in which R₂, R₃ and R₄ are the same or different and each are a linear or branched C₁-C₄ alkyl group, and R₁₃ represents a group —B(OH)₂ or a group 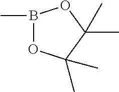
attached by the boron atom to the rest of the molecule.

13. Method according to claim 12, in which the compound of formula (IV) is prepared by halogenation of a compound of formula (III) 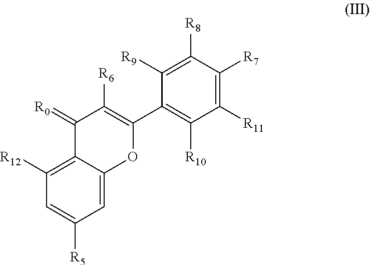
in which R₀, and R₅ to R₁₁ are as defined according to a claim 1 and R₁₂ is a hydroxyl group or a protected hydroxyl group, provided that at least one of R₅ and R₁₂ is a protected hydroxyl group.

14. Method according to claim 13, in which the compound of formula (III) is prepared by protection with the aid of a protective group of at least one of the two hydroxyl groups present on the benzene nucleus of the benzopyran ring of a compound of formula (II) 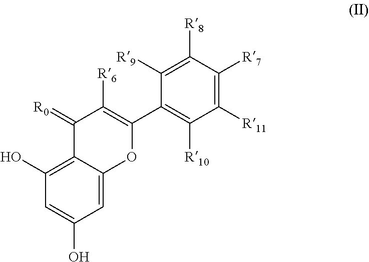
in which
R₀ is as defined according to claim 1; R′₆ to R′₁₁ represent, independently of one another, a hydrogen atom or a hydroxyl group.

15. Method for preparing a compound of formula (I) according to claim 9, comprising at least the following steps:
Step (A): Providing a compound of formula (II) 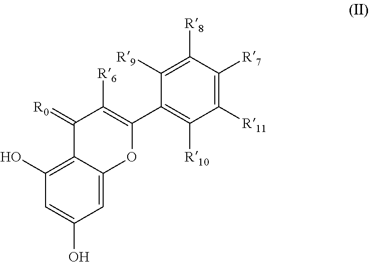
in which
R₀ is an oxygen atom or a group N—ORₐ;
R′₆ represents a hydrogen atom, a hydroxyl group, a group —O—P(═O)(OH)₂, or a sugar radical,
R′₇ represents a hydrogen atom, a hydroxyl group, a halogen atom, a linear or branched C₁-C₄ alkyl group, a linear or branched C₂-C₄ alkenyl group, a linear or branched C₂-C₄ alkynyl group, a triazolyl group, a group —O—Rb, a group N(Rc)(Rd), a group —C(═O)—N(Re)(Rf), or a group —O—C(═O)—N(Rg)(Rh); Ra, Rb, Rc, Rd, Re, Rf, Rg and Rh representing, independently of one another, a hydrogen atom or a linear or branched C₁-C₄ alkyl group,
R′₈ represents a hydrogen atom or a hydroxyl group,
R′₉ represents a hydrogen atom or a hydroxyl group,
R′₁₀ represents a hydrogen atom or a hydroxyl group,
and R′₁₁ represents a hydrogen atom or a hydroxyl group,
Step (B): Protection by means of a protective group, of at least one of the two hydroxyl groups present on the benzene nucleus of the benzopyran ring of the compound of formula (II) resulting from step (A) in order to obtain a compound of formula (III) 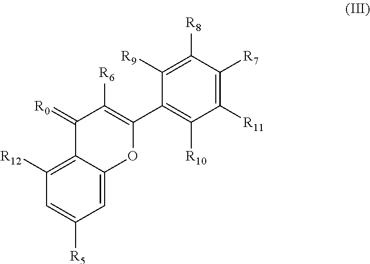
in which R₀ is an oxygen atom or a group N—ORₐ, and R₅ to R₁₁ are as defined according to claim 1 and R₁₂ is a hydroxyl group or a protected hydroxyl group as, provided that at least one of R₅ and R₁₂ is a protected hydroxyl group,
Step (C): Halogenation of the compound of formula (III) obtained in step (B) to obtain a compound of formula (IV) 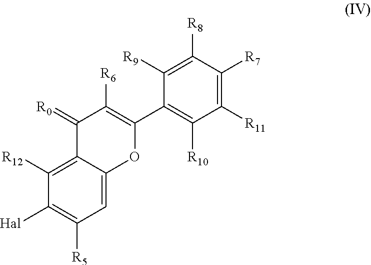
in which R₀, R₅ to R₁₁ and R₁₂ as defined above, provided that at least one of R₅ and R₁₂ is a protected hydroxyl group; and Hal represents a halogen atom;
Step (D): Geranylation of the compound of formula (IV) obtained in step (C) using a compound of formula (VIII) 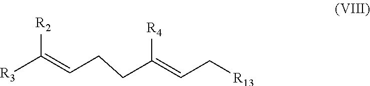
in which R₂ R₃ and R₄ are the same or different and are each a linear or branched C₁-C₄ alkyl group, and R₁₃ represents a group —B(OH)₂ or a group 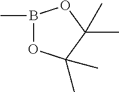
attached by the boron atom to the rest of the molecule, to obtain a compound of formula (V) 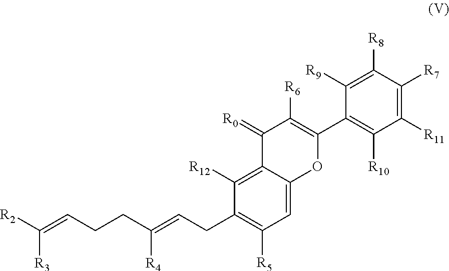
in which R₀, R₂ to R₁₁ and R₁₂ are as defined above, provided that at least one of R₅ and R₁₂ is a protected hydroxyl group
Step (E): Optionally dihydroxylation of the compound of formula (V) obtained in step (D) to obtain a compound of formula (VI) 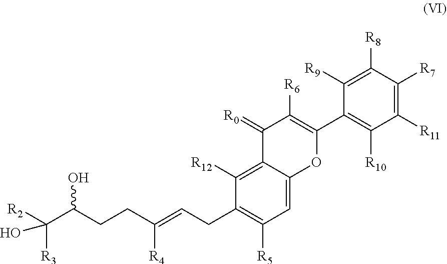
in which R₀, R₂ to R₁₁ and R₁₂ are as defined above, provided that at least one of R₅ and R₁₂ is a protected hydroxyl group, and the symbol [symbol] represents a single bond joining an asymmetric carbon atom to the group or atom in question, this bond being either in front of or behind the plane,
Step (F): Formation of the compound of formula (VII) starting from the compound of formula (VI) obtained in step (E) or starting from the compound of formula (V) obtained in step (D) 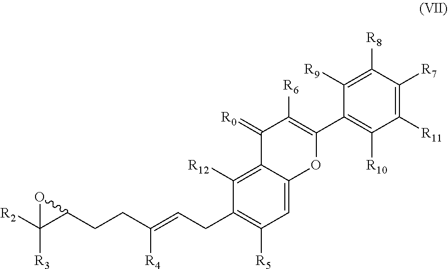
in which R₀, and R₂ to R₁₁ and R₁₂ are as defined above, provided that at least one of R₅ and R₁₂ is a protected hydroxyl group, and the symbol [symbol] represents a single bond joining an asymmetric carbon atom to the group or atom in question, this bond being either in front of or behind the plane,
Step (G):
(i) cyclization of the compound of formula (VII) obtained in step (F) to obtain a compound of formula (I), and
(ii) Optionally deprotection of at least one hydroxyl group protected by a protective group present in the compound obtained in step (i) to obtain a compound of formula (I).

16. Intermediates of formula (IV), (V), (VI) and (VII), their pharmaceutically acceptable salts, their hydrates, their solvates, as well as their tautomeric forms, stereoisomers, mixtures of stereoisomers, pure enantiomers and mixtures, whether or not racemic: 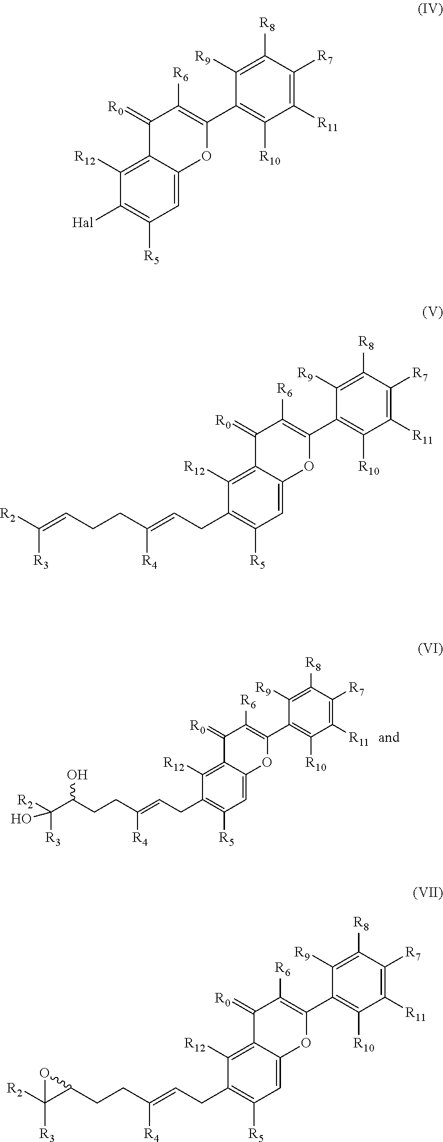
in which R₀, R₂, R₃, R₄, Hal, R₅, R₆, R₇, R₈, R₉, R₁₀, R₁₁ are as defined according to claim 1 and R₁₂ is a hydroxyl group or a protected hydroxyl group,
provided that at least one of R₅ and R₁₂ is a protected hydroxyl group, Hal is a halogen atom, and the symbol [symbol] represents a single bond joining an asymmetric carbon atom to the group or atom in question, this bond being either in front of or behind the plane, and
provided that:
for the compounds of formula (IV):
when R₇ is a benzyloxy group, R₅ is different from a methoxymethoxy group, a benzyloxy group, or a hydroxyl group,
when R₇ is a methoxy group, R₅ is different from a methoxy group, a beta-glucosyloxy group or a hexaacetyl-beta-rutinosyloxy group,
when R₇ is a hydrogen atom, R₅ is different from a methoxy group or a methoxymethoxy group, and
when R₇ is an ethoxy group, R₅ is different from an ethoxy group; and provided that
for the compounds of formula (V):
when R₇ is a methoxymethoxy group, R₆ is different from a hydrogen atom, and
when R₇ is a methoxy group, R₆ is different from a benzoyloxy group.

17. Intermediates according to claim 16, their pharmaceutically acceptable salts, their hydrates, their solvates, as well as their tautomeric forms, stereoisomers, mixtures of stereoisomers, pure enantiomers and mixtures, whether or not racemic, the compound being selected from:
5-hydroxy-6-iodo-3,7-bis(methoxymethoxy)-2-(4-(methoxymethoxy)phenyl)-4H-chromen-4-one,
5-hydroxy-6-iodo-7-(methoxymethoxy)-2-(4-(methoxymethoxy)phenyl)-4H-chromen-4-one,
(E)-6-(3,7-dimethylocta-2,6-dien-1-yl)-5,7-bis(methoxymethoxy)-2-phenyl-4H-chromen-4-one,
(Z)-6-(3,7-dimethylocta-2,6-dien-1-yl)-5,7-bis(methoxymethoxy)-2-phenyl-4H-chromen-4-one,
(E)-6-(3,7-dimethylocta-2,6-dien-1-yl)-5-hydroxy-7-(methoxymethoxy)-2-phenyl-4H-chromen-4-one,
(Z)-6-(3,7-dimethylocta-2,6-dien-1-yl)-5-hydroxy-7-(methoxymethoxy)-2-phenyl-4H-chromen-4-one,
(E)-6-(3,7-dimethylocta-2,6-dien-1-yl)-5-hydroxy-3,7-bis(methoxymethoxy)-2-(4-(methoxymethoxy)phenyl)-4H-chromen-4-one,
(Z)-6-(3,7-dimethylocta-2,6-dien-1-yl)-5-hydroxy-3,7-bis(methoxymethoxy)-2-(4-(methoxymethoxy)phenyl)-4H-chromen-4-one,
(S,E)-6-(6,7-dihydroxy-3,7-dimethyloct-2-in-1-yl)-5-hydroxy-7-(methoxymethoxy)-2-phenyl-4H-chromen-4-one,
(S,Z)-6-(6,7-dihydroxy-3,7-dimethyloct-2-in-1-yl)-5-hydroxy-7-(methoxymethoxy)-2-phenyl-4H-chromen-4-one,
(R,E)-6-(6,7-dihydroxy-3,7-dimethyloct-2-in-1-yl)-5-hydroxy-7-(methoxymethoxy)-2-phenyl-4H-chromen-4-one,
(R,Z)-6-(6,7-dihydroxy-3,7-dimethyloct-2-in-1-yl)-5-hydroxy-7-(methoxymethoxy)-2-phenyl-4H-chromen-4-one,
(S,E)-6-(6,7-dihydroxy-3,7-dimethyloct-2-in-1-yl)-5-hydroxy-3,7-bis(methoxymethoxy)-2-(4-(methoxymethoxy)phenyl)-4H-chromen-4-one,
(S,Z)-6-(6,7-dihydroxy-3,7-dimethyloct-2-in-1-yl)-5-hydroxy-3,7-bis(methoxymethoxy)-2-(4-(methoxymethoxy)phenyl)-4H-chromen-4-one,
(R,E)-6-(6,7-dihydroxy-3,7-dimethyloct-2-in-1-yl)-5-hydroxy-3,7-bis(methoxymethoxy)-2-(4-(methoxymethoxy)phenyl)-4H-chromen-4-one,
(R,Z)-6-(6,7-dihydroxy-3,7-dimethyloct-2-in-1-yl)-5-hydroxy-3,7-bis(methoxymethoxy)-2-(4-(methoxymethoxy)phenyl)-4H-chromen-4-one,
(S,E)-6-(6,7-dihydroxy-3,7-dimethyloct-2-in-1-yl)-5-hydroxy-7-(methoxymethoxy)-2-(4-(methoxymethoxy)phenyl)-4H-chromen-4-one,
(S,Z)-6-(6,7-dihydroxy-3,7-dimethyloct-2-in-1-yl)-5-hydroxy-7-(methoxymethoxy)-2-(4-(methoxymethoxy)phenyl)-4H-chromen-4-one,
(E)-6-(5-(3,3-dimethyloxiran-2-yl)-3-methylpent-2-in-1-yl)-5,7-bis(methoxymethoxy)-2-phenyl-4H-chromen-4-one (+/− racemic mixture),
(Z)-6-(5-(3,3-dimethyloxiran-2-yl)-3-methylpent-2-in-1-yl)-5,7-bis(methoxymethoxy)-2-phenyl-4H-chromen-4-one (+/− racemic mixture),
(R,E)-6-(5-(3,3-dimethyloxiran-2-yl)-3-methylpent-2-in-1-yl)-5-hydroxy-7-(methoxymethoxy)-2-phenyl-4H-chromen-4-one,
(R,Z)-6-(5-(3,3-dimethyloxiran-2-yl)-3-methylpent-2-in-1-yl)-5-hydroxy-7-(methoxymethoxy)-2-phenyl-4H-chromen-4-one,
(S,E)-6-(5-(3,3-dimethyloxiran-2-yl)-3-methylpent-2-in-1-yl)-5-hydroxy-7-(methoxymethoxy)-2-phenyl-4H-chromen-4-one,
(S,Z)-6-(5-(3,3-dimethyloxiran-2-yl)-3-methylpent-2-in-1-yl)-5-hydroxy-7-(methoxymethoxy)-2-phenyl-4H-chromen-4-one,
(R,E)-6-(5-(3,3-dimethyloxiran-2-yl)-3-methylpent-2-in-1-yl)-5-hydroxy-3,7-bis(methoxymethoxy)-2-(4-(methoxymethoxy)phenyl)-4H-chromen-4-one,
(R,Z)-6-(5-(3,3-dimethyloxiran-2-yl)-3-methylpent-2-in-1-yl)-5-hydroxy-3,7-bis(methoxymethoxy)-2-(4-(methoxymethoxy)phenyl)-4H-chromen-4-one,
(S,E)-6-(5-(3,3-dimethyloxiran-2-yl)-3-methylpent-2-in-1-yl)-5-hydroxy-3,7-bis(methoxymethoxy)-2-(4-(methoxymethoxy)phenyl)-4H-chromen-4-one,
(S,Z)-6-(5-(3,3-dimethyloxiran-2-yl)-3-methylpent-2-in-1-yl)-5-hydroxy-3,7-bis(methoxymethoxy)-2-(4-(methoxymethoxy)phenyl)-4H-chromen-4-one,
(R,E)-6-(5-(3,3-dimethyloxiran-2-yl)-3-methylpent-2-in-1-yl)-5-hydroxy-7-(methoxymethoxy)-2-(4-(methoxymethoxy)phenyl)-4H-chromen-4-one,
(R,Z)-6-(5-(3,3-dimethyloxiran-2-yl)-3-methylpent-2-in-1-yl)-5-hydroxy-7-(methoxymethoxy)-2-(4-(methoxymethoxy)phenyl)-4H-chromen-4-one.

18. Pharmaceutical composition comprising at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to, and at least one pharmaceutically acceptable excipient.

19. Medicament comprising at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1.

20. A method for treating conditions involving OSBP (oxysterol-binding protein) activation, comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to the subject in need thereof a pharmaceutical composition comprising the at lease one compound.

21. A method for treating cancers, neurodegenerative diseases, dyslipidemia, hypercholesterolemia, and/or viral diseases comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to a subject in need thereof a pharmaceutical composition comprising the at least one compound.

22. A method for treating a neurodegenerative disease which is selected from amyotrophic lateral sclerosis (ALS or Charcot disease), Alzheimer's disease, Parkinson's disease, multiple sclerosis, Huntington's disease (Huntington's chorea), and Niemann-Pick disease type C, comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to the subject in need thereof a pharmaceutical composition comprising the at least one compound.

23. A method for treating a cancer which is selected from breast cancer including triple-negative breast cancer, kidney cancer, head and neck cancer, prostate cancer, colorectal cancer, colon cancer, gallbladder cancer, biliary tract cancer (cholangiocarcinoma), gastrointestinal cancer, gastric cancer, hepatocellular carcinoma, lymphoma, lung cancer, small-cell lung cancer, nonsmall cell lung cancer, pancreatic cancer, pancreatic carcinoma, stomach cancer, brain cancer, metastases, leukemia, T-cell acute lymphoblastic leukemia, chronic myeloid leukemia, melanoma, and glioblastoma, comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to the subject in need thereof a pharmaceutical composition comprising the at least one compound.

24. A method for treating a viral disease which is selected from viral infections caused by RNA viruses, comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to the subject in need thereof a pharmaceutical composition comprising the at least one compound.

25. A method for treating a viral disease which is selected from dengue, Zika virus infection, influenza, viral pharyngitis, measles, AIDS, Chikungunya, yellow fever, poliomyelitis, hepatitis A, hepatitis C, hepatitis E, infection with SARS-CoV virus, infection with SARS-CoV-2 virus, and rubella, comprising administration to a subject in need thereof of at least one compound of formula (I), the pharmaceutically acceptable salts thereof, its hydrates, its solvates, as well as its tautomeric forms, stereoisomers, mixtures of stereoisomers, pure enantiomers and mixtures, whether or not racemic, as defined according to claim 1 or comprising administration to the subject in need thereof a pharmaceutical composition comprising the at least one compound.
